SEQUENCE LISTING (70 sequences)

SEQ ID NO: 1 - protein, 19 aa, Homo sapiens
RRRPKGRGKRRREKQRPTD

SEQ ID NO: 2 - protein, 13 aa, Homo sapiens
RKKKGGKNGKNRR

SEQ ID NO: 3 - protein, 12 aa, Homo sapiens
RRLRQRRRLRRE

SEQ ID NO: 4 - protein, 152 aa, Homo sapiens
RPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 5 - protein, 153 aa, Mus musculus
MRPSGKRPCKMQAFRIWDTNQKTFYLRNNQLIAGYLQGPNIKLEEKIDMVPIDLHSVFLGIHGGKLCLSCAKSGDDIKLQLEEVNITDLSKNKEEDKRFTFIRSEKGPTTSFESAACPGWFLCTTLEADRPVSLTNTPEEPLIVTKFYFQEDQ

SEQ ID NO: 6 - protein, 170 aa, Homo sapiens
  SITE (142)..(170): ECM binding peptide
MPVMRLFPCFLQLLAGLALPAVPPQQWALSAGNGSSEVEVVPFQEVWGRSYCRALERLVDVVSEYPSEVEHMFSPSCVSLLRCTGCCGDENLHCVPVETANVTMQLLKIRSGDRPSYVELTFSQHVRCECRPLREKMKPERRRPKGRGKRRREKQRPTDCHLCGDAVPRR

SEQ ID NO: 7 - protein, 242 aa, Homo sapiens
  SITE (213)..(242): ECM binding peptide
MPVMRLFPCFLQLLAGLALPAVPPQQWALSAGNGSSEVEVVPFQEVWGRSYCRALERLVDVVSEYPSEVEHMFSPSCVSLLRCTGCCGDENLHCVPVETANVTMQLLKIRSGDRPSYVELTFSQHVRCECRHSPGRQSPDMPGDFRADAPSFLPPRRSLPMLFRMEWGCALTGSQSAVWPSSPVPEEIPRMHPGRNGKKQQRKPLREKMKPERRRPKGRGKRRREKQRPTDCHLCGDAVPRR

SEQ ID NO: 8 - protein, 30 aa, Homo sapiens
RRRPKGRGKRRREKQRPTDCHLCGDAVPRR

SEQ ID NO: 9 - protein, 18 aa, Mus musculus
RRKTKGKRKRSRNSQTEE

SEQ ID NO: 10 - protein, 19 aa, Canis lupus familiaris
RRRPKGRGKRKREKQRPTD

SEQ ID NO: 11 - protein, 19 aa, Hylobates moloch
RRRPKGRGKRRREKQKPTD

SEQ ID NO: 12 - protein, 19 aa, Callithrix jacchus
RRRPKGRGKRRREKQRPKD

SEQ ID NO: 13 - protein, 19 aa, Galeopterus variegatus
RRRPKGRGKRKRDKQRPTD

SEQ ID NO: 14 - protein, 19 aa, Carlito syrichta
RRRPKGRGKRKREKQKPTD

SEQ ID NO: 15 - protein, 19 aa, Lagenorhynchus obliquidens
RRRPKGRGKRKREKQRHTD

SEQ ID NO: 16 - protein, 19 aa, Suricata suricatta
RRRPKGRGKRKREKQRPRD

SEQ ID NO: 17 - protein, 19 aa, Trichechus manatus latirostris
RRRSRGRGKRKREKQRPTD

SEQ ID NO: 18 - protein, 17 aa, Pelodiscus sinensis
RRRPKGQGKRRREKQRP

SEQ ID NO: 19 - protein, 19 aa, Eptesicus fuscus
RRRPKGSGKRKKEKQRPTD

SEQ ID NO: 20 - protein, 17 aa, Chrysemys picta bellii
RRRPKGQGKRKREKQRP

SEQ ID NO: 21 - protein, 19 aa, Ailuropoda melanoleuca
RRRHKGRRKRKREKQRPTD

SEQ ID NO: 22 - protein, 18 aa, Fukomys damarensis
RRRPKVRGKRKREKQKPT

SEQ ID NO: 23 - protein, 19 aa, Podarcis muralis
RRRPKGRSKRKRAKQRPKD

SEQ ID NO: 24 - protein, 19 aa, Lacerta agilis
RRRPKGRSKRKRAKQRPKD

SEQ ID NO: 25 - protein, 19 aa, Ictidomys tridecemlineatus
RRRLKGRGKRKKEKQRSTD

SEQ ID NO: 26 - protein, 18 aa, Cavia porcellus
RRRPKVRGKRKRENQKPT

SEQ ID NO: 27 - protein, 19 aa, Echinops telfairi
RRRYRGRGKRKREKQRATD

SEQ ID NO: 28 - protein, 16 aa, Electrophorus electricus
RRRPKGRGRKRKEKQR

SEQ ID NO: 29 - protein, 18 aa, Phyllostomus discolor
RRRNKGSGKRKKEKQRPT

SEQ ID NO: 30 - protein, 19 aa, Anas platyrhynchos
RRRPKGRGKRRGEKKRRKD

SEQ ID NO: 31 - protein, 19 aa, Paramormyrops kingsleyae
RRRQKGRGRKRKDKQRPKD

SEQ ID NO: 32 - protein, 16 aa, Strigops habroptila
RRRPKGRGKRRQDKMR

SEQ ID NO: 33 - protein, 18 aa, Ornithorhynchus anatinus
RRRSKDRGKRKRERPRPT

SEQ ID NO: 34 - protein, 16 aa, Amazona aestiva
RRRPKGRGKRRQERTR

SEQ ID NO: 35 - protein, 16 aa, Pygocentrus nattereri
RRRPRGRGRKRKEKQR

SEQ ID NO: 36 - protein, 19 aa, Sarcophilus harrisii
RRRFKGRGKRKRDKQRTKD

SEQ ID NO: 37 - protein, 19 aa, consensus sequence
RRRPKGRGKRKREKQRPTD

SEQ ID NO: 38 - protein, 20 aa, Homo sapiens
RRRPKGRGKRRREKQRPTDC

SEQ ID NO: 39 - protein, 21 aa, Homo sapiens
RRRPKGRGKRRREKQRPTDCH

SEQ ID NO: 40 - protein, 23 aa, Homo sapiens
RRRPKGRGKRRREKQRPTDCHLC

SEQ ID NO: 41 - protein, 24 aa, Homo sapiens
RRRPKGRGKRRREKQRPTDCHLCG

SEQ ID NO: 42 - protein, 25 aa, Homo sapiens
RRRPKGRGKRRREKQRPTDCHLCGD

SEQ ID NO: 43 - protein, 26 aa, Homo sapiens
RRRPKGRGKRRREKQRPTDCHLCGDA

SEQ ID NO: 44 - protein, 27 aa, Homo sapiens
RRRPKGRGKRRREKQRPTDCHLCGDAV

SEQ ID NO: 45 - protein, 28 aa, Homo sapiens
RRRPKGRGKRRREKQRPTDCHLCGDAVP

SEQ ID NO: 46 - protein, 29 aa, Homo sapiens
RRRPKGRGKRRREKQRPTDCHLCGDAVPR

SEQ ID NO: 47 - protein, 14 aa, Homo sapiens
RKKKGGKNGKNRRN

SEQ ID NO: 48 - protein, 15 aa, Homo sapiens
RKKKGGKNGKNRRNR

SEQ ID NO: 49 - protein, 16 aa, Homo sapiens
RKKKGGKNGKNRRNRK

SEQ ID NO: 50 - protein, 17 aa, Homo sapiens
RKKKGGKNGKNRRNRKK

SEQ ID NO: 51 - protein, 18 aa, Homo sapiens
RKKKGGKNGKNRRNRKKK

SEQ ID NO: 52 - protein, 19 aa, Homo sapiens
RKKKGGKNGKNRRNRKKKN

SEQ ID NO: 53 - protein, 20 aa, Homo sapiens
RKKKGGKNGKNRRNRKKKNP

SEQ ID NO: 54 - protein, 21 aa, Homo sapiens
RKKKGGKNGKNRRNRKKKNPC

SEQ ID NO: 55 - protein, 22 aa, Homo sapiens
RKKKGGKNGKNRRNRKKKNPCN

SEQ ID NO: 56 - protein, 23 aa, Homo sapiens
RKKKGGKNGKNRRNRKKKNPCNA

SEQ ID NO: 57 - protein, 23 aa, Homo sapiens
KPKRKKKGGKNGKNRRNRKKKNP

SEQ ID NO: 58 - protein, 22 aa, Homo sapiens
PKRKKKGGKNGKNRRNRKKKNP

SEQ ID NO: 59 - protein, 21 aa, Homo sapiens
KRKKKGGKNGKNRRNRKKKNP

SEQ ID NO: 60 - protein, 171 aa, Artificial Sequence
  Fusion protein
  SITE (153)..(171)
  SITE (153)..(171): PIGF 123-141 (SEQ ID NO 1)
RPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDERRRPKGRGKRRREKQRPTD

SEQ ID NO: 61 - protein, 183 aa, Artificial Sequence
  Fusion protein
  SITE (1)..(11): Histidine tag
  SITE (165)..(183): PIGF 123-141 (SEQ ID NO 1)
MNHKVHHHHHHMRPSGKRPCKMQAFRIWDTNQKTFYLRNNQLIAGYLQGPNIKLEEKIDMVPIDLHSVFLGIHGGKLCLSCAKSGDDIKLQLEEVNITDLSKNKEEDKRFTFIRSEKGPTTSFESAACPGWFLCTTLEADRPVSLTNTPEEPLIVTKFYFQEDQRRRPKGRGKRRREKQRPTD

SEQ ID NO: 62 - protein, 18 aa, Homo sapiens
RRLRQRRRLRRERVRAQP

SEQ ID NO: 63 - protein, 15 aa, Homo sapiens
RRLRQRRRLRRERVR

SEQ ID NO: 64 - protein, 16 aa, Homo sapiens
RRRPKGRGKRRREKQR

SEQ ID NO: 65 - protein, 15 aa, Homo sapiens
KKDRARQEKKSVRGK

SEQ ID NO: 66 - protein, 9 aa, Homo sapiens
RRLRQRRRL

SEQ ID NO: 67 - protein, 6 aa, Homo sapiens
RQRRRL

SEQ ID NO: 68 - protein, 26 aa, Homo sapiens
KPKRKKKGGKNGKNRRNRKKKNPCNA

SEQ ID NO: 69 - protein, 12 aa, Homo sapiens
GGKNGKNRRNRK

SEQ ID NO: 70 - protein, 10 aa, Homo sapiens
RKKKGGKNGK

(19) United States
(12) Patent Application Publication
(10) Pub. No.: US 2023/0272045 A1
(43) Pub. Date: Aug. 31, 2023

(54) IL-1 RECEPTOR ANTAGONIST (IL-1 RA) FUSION PROTEINS BINDING TO THE EXTRACELLULAR MATRIX

(71) Applicant: MONASH UNIVERSITY, Clayton, Victoria (AU)

(72) Inventors: Mikaël MARTINO, Clayton, VIC (AU); Ziad JULIER, Clayton, VIC (AU)

(73) Assignee: MONASH UNIVERSITY, Clayton, Victoria (AU)

(21) Appl. No.: 18/001,480

(22) PCT Filed: Jun. 11, 2021
(86) PCT No.: PCT/AU2021/050598

§ 371 Date: Dec. 12, 2022

(30) Foreign Application Priority Data
Jun. 12, 2020 (AU) 2020901944

(51) Int. Cl. C07K 14/715; C07K 14/475; C12N 15/62; A61P 17/02; C07K 14/71

(52) U.S. Cl. CPC C07K 14/7155; C07K 14/475; C12N 15/62; A61P 17/02; C07K 14/71; C07K 2319/33

(57) ABSTRACT

The present invention provides a fusion protein comprising interleukin-1 receptor antagonist (IL-1Ra) and an extracellular matrix (ECM) binding peptide which specifically binds to one or more or all extracellular matrix proteins selected from the group consisting of fibrinogen, fibronectin, vitronectin, tenascin C and heparan sulfate and use of the fusion protein to treat conditions in which administration of IL-1Ra is beneficial or in which IL-1R1 signalling needs to be dampened, to enhance tissue regeneration, particularly bone regeneration and/or wound repair or for treating wounds, burns and muscle, cartilage, tendon and bone disorders, to enhance the regenerative activity of growth factor administration or to reduce inflammation or desensitisation of a cell to growth factor stimulation.

### FIELD

The invention relates to fusion proteins and uses thereof in wound healing and tissue regeneration.

### BACKGROUND

All references, including any patents or patent application, cited in this specification are hereby incorporated by reference to enable full understanding of the invention. Nevertheless, such references are not to be read as constituting an admission that any of these documents forms part of the common general knowledge in the art, in Australia or in any other country. The discussion of the references states what their authors assert, and the applicants reserve the right to challenge the accuracy and pertinency of the cited documents.

IL-1 receptor antagonist (IL-1Ra or IRAP) is the natural antagonist of the proinflammatory cytokine family interleukin-1 (IL-1) that initiates and regulates inflammatory responses. IL-1 can stimulate lymphocytes and macrophages, activate phagocytes, increase prostaglandin production, contribute to degeneration of bone joints and increase bone marrow cell proliferation. IL-1 is involved in many chronic inflammatory conditions.

Treatment of IL-1 related conditions through the administration of IL-1Ra has been extensively studied in both in vitro and in animal models. These models include those for infection, local inflammation, acute or chronic lung injury, metabolic dysfunction, autoimmune disease, immune-mediated disease, malignant disease, and host responses. In addition, human recombinant IL-1Ra has been administered to humans in clinical trials for rheumatoid arthritis, septic shock, steroid resistant graft versus host disease, acute myeloid leukemia, and chronic myelogenous leukemia.

Compositions of IL-1Ra are known in the art. However, many such compositions are associated with issues regarding stability and half-life of IL-1Ra as well as the amount and rate of IL-1Ra provided at the intended site of action.

Recombinant IL-1 Ra (Anakinra, Kineret) is approved for the treatment of rheumatoid arthritis and neonatal-onset multisystem inflammatory disease. It needs to be used at very high doses (>100 mg per injection) with multiple bulk administrations. Its use as an immunosuppressant is reportedly linked to infections and immunogenicity.

Considering the potential of IL-1Ra to treat many conditions, better delivery systems need to be developed to allow precise localization and retention of low doses of IL-1Ra where required. Accordingly, improved methods of delivering IL-1ra are desirable and would be useful in treating conditions and pathologies mediated by the interleukin-1 receptor.

An embodiment of the present invention seeks to provide a controlled release form of IL-1Ra capable of being retained at the desired site of action.

It is a further aim of an embodiment of the present invention to determine further conditions that may be treated by administration of IL-1Ra, particularly a controlled release form of IL-1Ra.

### SUMMARY

A first aspect provides a fusion protein comprising interleukin-1 receptor antagonist (IL-1Ra) and an extracellular matrix (ECM) binding peptide which specifically binds to one or more or all extracellular matrix proteins selected from the group consisting of fibrinogen, fibronectin, vitronectin, tenascin C and heparan sulfate.

In one embodiment the ECM binding peptide comprises a heparin binding domain of placental growth factor comprising the amino acid sequence provided as SEQ ID NO: 1 or conservative variants thereof.

RRRPKGRGKRRREKQRPTD SEQ ID NO: 1

In one embodiment the ECM binding peptide comprises a peptide from amphiregulin (AREG) comprising the amino acid sequence provided as SEQ ID NO: 2 or conservative variations thereof.

RKKKGGKNGKNRR SEQ ID NO: 2

In one embodiment the ECM binding peptide comprises a peptide from neurturin (NRTN) comprising the amino acid sequence provided as SEQ ID NO: 3 or conservative variations thereof.

RRLRQRRRLRRE SEQ ID NO: 3

A second aspect provides a nucleic acid molecule encoding the fusion protein of the first aspect.

A third aspect provides a vector comprising the nucleic acid molecule of the second aspect.

A fourth aspect comprises a cell or a non-human organism transformed or transfected with the nucleic acid molecule of the second aspect or the vector of the third aspect.

A fifth aspect provides a method of making the fusion protein of the first aspect, the method comprising culturing the cell of the fourth aspect under conditions to produce the fusion protein and recovering the fusion protein.

A sixth aspect provides a fusion protein when produced by the method of the fifth aspect.

A seventh aspect provides a pharmaceutical or veterinary composition comprising the fusion protein of the first aspect, the nucleic acid molecule of the second aspect, the vector of the third aspect or the cell or non-human organism of the fourth aspect, optionally with one or more excipient and/or carriers.

An eighth aspect provides a method of treatment of a condition in which IL-1Ra administration is beneficial or in which IL-1R1 signalling needs to be dampened, the method comprising administering to a subject in need thereof the fusion protein of the first aspect or the sixth aspect, the nucleic acid molecule of the second aspect, the vector of the third aspect, the cell or non-human organism of the fourth aspect or the pharmaceutical or veterinary composition of the seventh aspect.

An alternative form of the eighth aspect provides a composition for treatment of a condition in which II-1Ra administration is beneficial or in which IL-1R1 signalling needs to be dampened, the composition comprising the fusion protein of the first aspect or the sixth aspect, the nucleic acid molecule of the second aspect, the vector of the third aspect, the cell or non-human organism of the fourth aspect or the pharmaceutical or veterinary composition of the seventh aspect.

A further alternative form of the eighth aspect provides use of the fusion protein of the first aspect or the sixth aspect, the nucleic acid molecule of the second aspect, the vector of the third aspect, the cell or non-human organism of the fourth aspect or the pharmaceutical or veterinary composition of the seventh aspect in the manufacture of a medicament for treating a condition in which IL-1Ra administration is beneficial or in which IL-1R1 signalling needs to be dampened.

In an embodiment of the eighth aspect the condition in which IL-1Ra administration is beneficial or in which IL-1R1 signalling needs to be dampened is a condition requiring tissue regeneration, particularly bone regeneration and/or wound repair. In one embodiment of the eighth aspect the condition is a wound, burn or muscle condition or a cartilage, tendon or bone disorders. In a particular embodiment the condition is wound healing in diabetics. In one embodiment the condition is an inflammatory condition.

A ninth aspect provides a fusion protein comprising IL-1Ra fused to PIGF₁₂₃₋₁₄₁, SEQ ID NO: 1.

A tenth aspect provides a fusion protein comprising IL-1Ra fused to AREG₁₂₆₋₁₃₈, SEQ ID NO: 2.

An eleventh aspect provides a fusion protein comprising IL-1Ra fused to NRTN₁₄₆₋₁₅₇, SEQ ID NO: 3.

A twelfth aspect provides a method of enhancing tissue regeneration, particularly bone regeneration and/or wound repair or for treating wounds, burns and muscle, cartilage, tendon and bone disorders, the method comprising administering the IL-1Ra fusion protein of the ninth, tenth or eleventh aspect.

An alternative form of the twelfth aspect provides the IL-1Ra fusion protein of the ninth, tenth or eleventh aspect for use in enhancing tissue regeneration, particularly bone regeneration and/or wound repair or for treating wounds, burns and muscle, cartilage, tendon and bone disorders.

A further alternative form of the twelfth aspect provides use of the IL-1Ra fusion protein of the ninth, tenth or eleventh aspect in the manufacture of a medicament for enhancing tissue regeneration, particularly bone regeneration and/or wound repair or for treating wounds, burns and muscle, cartilage, tendon and bone disorders.

A thirteenth aspect provides a method of enhancing the regenerative activity of growth factor administration, the method comprising administering the growth factor with the IL-1Ra fusion protein of the first aspect, sixth or ninth, tenth or eleventh aspect, the nucleic acid molecule of the second aspect, the vector of the third aspect, the cell or non-human organism of the fourth aspect or the pharmaceutical or veterinary composition of the seventh aspect.

An alternative form of the thirteenth aspect provides a fusion protein of the first, sixth or ninth, tenth or eleventh aspect, the nucleic acid molecule of the second aspect, the vector of the third aspect, the cell or non-human organism of the fourth aspect or the pharmaceutical or veterinary composition of the seventh aspect for administering to a subject being treated with a growth factor, to enhance the regenerative activity of the growth factor.

A further alternative form of the thirteenth aspect provides use of a fusion protein of the first, sixth or ninth, tenth or eleventh aspect, the nucleic acid molecule of the second aspect, the vector of the third aspect, the cell or non-human organism of the fourth aspect or the pharmaceutical or veterinary composition of the seventh aspect for in the manufacture of a medicament for administering to a subject being treated with a growth factor, to enhance the regenerative activity of the growth factor.

A fourteenth aspect provides a method for reducing inflammation or desensitisation to growth factor stimulation, the method comprising administering the growth factor together with the IL-1Ra fusion protein of the first, sixth or ninth, tenth or eleventh aspect, the nucleic acid molecule of the second aspect, the vector of the third aspect, the cell or non-human organism of the fourth aspect or the pharmaceutical or veterinary composition of the seventh aspect.

An alternative form of the fourteenth aspect provides a fusion protein of the first, sixth or ninth, tenth or eleventh, the nucleic acid molecule of the second aspect, the vector of the third aspect, the cell or non-human organism of the fourth aspect or the pharmaceutical or veterinary composition of the seventh aspect for administering to a subject being treated with a growth factor, to reduce inflammation or desensitisation to growth factor stimulation.

A further alternative form of the fourteenth aspect provides use of a fusion protein of the first, sixth or ninth, tenth or eleventh aspect, the nucleic acid molecule of the second aspect, the vector of the third aspect, the cell or non-human organism of the fourth aspect or the pharmaceutical or veterinary composition of the seventh aspect for in the manufacture of a medicament for administering to a subject being treated with a growth factor, to reduce inflammation or desensitisation to growth factor stimulation.

A fifteenth aspect provides a method for treating wounds, particularly diabetic skin wounds, the method comprising administering the IL-1Ra fusion protein of the first, sixth or ninth, tenth or eleventh aspect, in which the fusion protein is more capable of restoring a healing microenvironment in chronic wounds than IL-1Ra or saline.

An alternative form of the fifteenth aspect provides the IL-1Ra fusion protein of the first, sixth or ninth, tenth or eleventh aspect for treating wounds, particularly diabetic skin wounds, in which the fusion protein is more capable of restoring a healing microenvironment in chronic wounds than IL-1Ra or saline.

A further alternative form of the fifteenth aspects provides use of the IL-1Ra fusion protein of the first, sixth or ninth, tenth or eleventh aspect in the manufacture of a medicament for treating wounds, particularly diabetic skin wounds, in which the fusion protein is more capable of restoring a healing microenvironment in chronic wounds than IL-1Ra or saline.

In one embodiment the ability of the fusion protein of the first, sixth or ninth, tenth or eleventh aspect to restore a healing microenvironment in chronic wounds more than IL-1Ra or saline in accordance with the fifteen aspect, is evidenced by at least one of increased clearance of neutrophils (CD11b+, Ly6G+ cells), increased accumulation of macrophages (F4/80+, CD11 b+ cells), increased expression of CD206, reducing pro-inflammatory factor concentrations, increasing ant-inflammatory factor concentrations, decreasing MMP-2 and 9 concentrations, increasing metallopeptidase inhibitor TIMP-1 concentrations, increasing fibroblast growth factor-2 (FGF-2), PDGF-BB, and vascular endothelial growth factor-A (VEGF-A) concentrations or decreasing SA-β-gal activity in wound fibroblasts compared to IL-1Ra or saline in a diabetic mice (Lepr^{db/db}) full-thickness wound healing model. The Lepr^{db/db} diabetic mouse model of full-thickness wound healing is described in Chen, H. et al., (1996) Cell 84(3): 491-5 and Sullivan, S. R. et al., (2004) Plastic and Reconstructive Surgery vol 113, issue 3; p 953-960.

A sixteenth aspect provides a method of enhancing tissue regeneration, particularly bone regeneration the method comprising administering the IL-1Ra fusion protein of the first, sixth or ninth, tenth or eleventh aspect, in which the fusion protein has greater regenerative capacity than IL-1Ra or saline.

An alternative form of the sixteenth aspect provides the IL-1Ra fusion protein of the first, sixth or ninth, tenth or eleventh aspect for tissue regeneration, particularly bone regeneration, in which the fusion protein has greater regenerative capacity than IL-1Ra or saline.

A further alternative form of the sixteenth aspect provides use of the IL-1Ra fusion protein of the first, sixth or ninth, tenth or eleventh aspect in the manufacture of a medicament for use in tissue regeneration, particularly bone regeneration, in which the fusion protein has greater regenerative capacity than IL-1Ra or saline.

In one embodiment of the sixteenth aspect the IL-1Ra fusion protein of the first, sixth or ninth, tenth or eleventh aspect is capable of increasing the tissue regenerating, particularly bone regenerating, capacity of growth factors such as BMP-2 and PDGF-BB.

In one embodiment the ability of the fusion protein of the first, sixth or ninth, tenth or eleventh aspect to increase the tissue regenerating, particularly bone regenerating, capacity of growth factors such as BMP-2 and PDGF-BB is evidenced by at least one of decreasing Smurf2 expression, maintaining or increasing Smad1/5/8 levels to enhanced BMP-2-driven differentiation or decreasing PHLPPs or decreasing AKT dephosphorylation to improve proliferation and migration responses induced by PDGF-BB, for example using methods described in relation to Example 3. Methods to detect Smurf2 expression and Smad1/5/8 levels are described in Zhang. Y (2001) PNAS, 98, 974-9. Methods to detect PHLPPs are described in Sierecki, E. et al., (2010) J Med Chem 53, 6899-6911. Methods to detect AKT proliferation are described in Manning, B. D. and Cantley, L. C. (2007) Cell 129, 1261-1274.

It has previously been described (see for example U.S. Pat. No. 9,879,062 B2) that the heparin binding domain of placental growth factor (PIGF₁₂₃₋₁₅₂) exhibits specific binding activity for the extracellular matrix (ECM). Binding of fusions proteins of glutathione S-transferase (GST) and PIGF₁₂₃₋₁₅₂ or its fragments PIGF₁₂₃₋₁₄₄, PIGF₁₂₃₋₁₄₁, PIGF₁₂₃₋₁₃₇, PIGF₁₃₀₋₁₃₇ and PIGF₁₂₃₋₁₃₂ to ECM proteins fibronectin and collagen I identified that PIGF₁₂₃₋₁₄₄ had the strongest binding affinity for fibronectin and significantly stronger binding for collagen 1 than any other fusion protein tested. Fusions proteins comprising PIGF₁₂₃₋₁₄₄ fused to growth factors were made because it is well documented that interaction of growth factors with the ECM naturally plays a role in growth factor signalling and it was desirable to determine if growth factors could be engineered to bind the ECM and to determine the effect, if any on growth factor activity. Fusion proteins comprising PIGF₁₂₃₋₁₄₄ fused to growth factors including VEGF, PDGF-BB, BMP-2, IGF-1, BDNF, NT, TGF-β1 and TGF-β2 were observed to retain their wild-type activity in vitro and bound to and were retained by ECM molecules in vivo.

The ability of IL-1Ra to bind ECM proteins is poorly documented and ECM interactions are not known to be involved in the activity of IL-1Ra. Accordingly, it was not predictable that fusing the heparin binding domain of PIGF to IL-1Ra would have any impact on the activity of IL-1Ra. Regardless, the inventors determined the smallest ECM-binding sequence from PIGF₁₂₃₋₁₅₂, by producing seven truncated version of PIGF₁₂₃₋₁₅₂ and testing their binding to common ECM proteins (fibronectin, vitronectin, tenascin C, and fibrinogen) and heparan sulfate. They found that the ECM-binding sequence, PIGF₁₂₃₋₁₄₁, strongly binds all ECM protein tested as well as heparan sulfate. The binding affinity of PIGF₁₂₃₋₁₄₁ was significantly better than the other truncated versions of PIGF₁₂₃₋₁₅₂ including PIGF₁₂₃₋₁₄₄ and PIGF₁₂₃₋₁₅₂. This was not predictable from the prior art, which suggests that PIGF₁₂₃₋₁₄₄ would be have the best binding affinity.

The inventors then engineered IL-1Ra with PIGF₁₂₃₋₁₄₁ at its C-terminus to generate IL-1Ra/PIGF₁₂₃₋₁₄₁. Fusing PIGF peptide₁₂₃₋₁₄₁ to IL-1Ra provided very strong binding (i.e. super-affinity) to all ECM proteins tested (fibronectin, vitronectin, tenascin C, and fibrinogen), with 4 to 100-fold increase in affinity. Moreover, PIGF₁₂₃₋₁₄₁-fused IL-1Ra was strongly retained in fibrin, while wild-type IL-1Ra was quickly released. Although IL-1Ra/PIGF₁₂₃₋₁₄₁ was retained in fibrin, it was gradually released in the presence of the protease plasmin which cleaves fibrin fibers and PIGF₁₂₃₋₁₄₁, providing a controlled release composition. Notably, IL-1Ra was not compromised by the fusion with PIGF₁₂₃₋₁₄₁, since wild-type IL-1Ra and IL-1Ra/PIGF₁₂₃₋₁₄₁ displayed comparable ability to inhibit the macrophage response to IL-1β.

Compared to wild-type the super-affinity IL-1Ra/PIGF₁₂₃₋₁₄₁ fusion protein showed much longer retention after intradermal administration in vivo, with about 50% retained after 5 days.

Surprisingly and significantly, compared to wild-type the super affinity IL-1Ra fusion protein showed significantly more closure of diabetic wounds, characterised by the extent of re-epithelisation, with nearly 100% closure 9 days after treatment, while wounds treated with wild-type were still largely open. It appears that the fusion protein was able to restore the healing microenvironment in chronic wounds.

Surprisingly, treatment of wounds with the super-affinity IL-1Ra fusion protein promoted faster clearance of neutrophils (CD11 b+, Ly6G+ cells) and accumulation of more macrophages (F4/80+, CD11b+ cells), compared to treatment with wild-type IL-1Ra or saline. Moreover, the expression levels of CD206—a marker for M2-like macrophages—were significantly higher in the super affinity IL-1Ra fusion protein treated group, indicating that wound macrophages were likely more anti-inflammatory. Remarkably, compared to treatment with saline and IL-1Ra, the super-affinity IL-1Ra fusion protein significantly reduced pro-inflammatory factor concentrations in the wounds while increasing the concentrations of the ant-inflammatory factors. Delivering the super-affinity IL-1Ra fusion protein significantly decreased the levels of MMP-2 and 9 but increased the levels of the MMP inhibitor metallopeptidase inhibitor TIMP-1. Furthermore, delivering the super-affinity IL-1Ra fusion protein significantly enhanced the concentration of the pro-healing factors fibroblast growth factor-2 (FGF-2), PDGF-BB, and vascular endothelial growth factor-A (VEGF-A) which are key wound healing growth factors secreted by macrophages and other cells, compared to saline and IL-1Ra. Lastly, the super-affinity IL-1Ra fusion protein decreased SA-β-gal activity in wound fibroblasts such that 9 days post-treatment wound fibroblast displayed a level of SA-β-gal activity similar to dermal fibroblasts found in uninjured skin. None of this was predictable from the prior art.

The effect of the super-affinity IL-1Ra fusion protein was also tested on bone regeneration as IL-1Ra is known to have some regenerative capacity. Surprisingly, the super-affinity IL-1Ra fusion protein had greater regenerative capacity compared to its wild-type form but more surprising was that co-delivering BMP-2 or PDGF-BB with the IL-1Ra fusion protein significantly stimulates superior bone regeneration compared to the delivery of BMP-2 or PDGF-BB alone. Surprisingly, it appears that inhibiting IL-1R1 signalling with the super-affinity IL-1Ra fusion protein enhances the bone regenerative response to BMP-2 and PDGF-BB.

In the context of bone regeneration, BMP-2 and PDGF-BB are well-known to act on bone-resident MSCs and osteoblasts to promote new bone formation. BMP-2 promotes differentiation, while PDGF-BB promotes chemotaxis and proliferation. Here, we found that IL-1R1 signalling inhibits the fundamental morphogenic effects triggered by both growth factors. Mechanistically, we propose that exposure to IL-1R1 signalling makes MSCs and osteoblasts less responsive to BMP-2 and PDGF-BB, by two mechanisms. In the context of BMP-2, IL-1R1 signalling increases Smurf2 expression and promotes Smad1/5/8 degradation, which results in an impairment of BMP-2-driven differentiation, due to lower Smad1/5/8 levels. In line with this finding, it has been shown that NF-κB—the main transcription factor activated by IL-1R1 signalling—inhibits osteogenic differentiation of MSCs by promoting β-catenin degradation via Smurf2. In the context of PDGF-BB, IL-1R1 signalling increases expression of PHLPPs which drives quicker Akt dephosphorylation and impairs proliferation and migration responses which are normally induced by PDGF-BB. Similarly, it has been shown that inflammatory mediators signalling via NF-κB also enhance PHLPP1 in human chondrocytes. Accordingly administer the super-affinity IL-1Ra fusion protein is expected to decrease Smurf2 expression and maintain or increase Smad1//5/8 levels to enhanced BMP-2-driven differentiation. In addition, administering the super-affinity IL-1Ra fusion protein is expected to decrease PHLPPs to decrease AKT dephosphorylation to improve proliferation and migration responses induced by PDGF-BB.

Notably, because Smad1/5/8 and Akt are critical in the signalling of many growth factors, activation of IL-1R1 may not only inhibit the activity of recombinant BMP-2 and PDGF-BB, but also several other potential therapeutics such as BMPs and growth factors in the vascular, fibroblast, and epidermal growth factors families. Administration of the super-affinity fusion protein in addition to these therapeutics may overcome the dampening of their activity by IL-1R1 activation.

Macrophage polarization from an inflammatory to an anti-inflammatory state is well-known to be important for tissue healing. Interestingly, mice treated with the super-affinity IL-1Ra fusion protein displayed a higher percentage of anti-inflammatory-like macrophages which are commonly characterized by the surface expression of CD206. This suggests that, in addition to restoring BMP-2 and PDGF-BB signalling in MSCs and osteoblasts, the super-affinity IL-1Ra fusion protein may also promote bone regeneration by supporting macrophages polarization towards an anti-inflammatory phenotype. None of this was predictable from the prior art.

### BRIEF DESCRIPTION OF THE DRAWINGS

Embodiments of the invention will now be described by way of example only with reference to the accompanying drawings in which:

FIG. 1 illustrates the design and testing of IL-1Ra fusion proteins with super-affinity to the ECM. FIG. 1A shows the amino acid sequences of PIGF₁₂₃₋₁₅₂, PIGF₁₂₃₋₁₄₄, PIGF₁₂₃₋₁₄₁, PIGF₁₂₃₋₁₃₇, PIGF₁₂₃₋₁₃₄, PIGF₁₂₃₋₁₄₀, and PIGF₁₃₀₋₁₃₇. FIG. 1B graphs show signals given when detecting glutathione S-transferase (GST) when ELISA plates coated with ECM proteins were incubated with PIGF fragments fused to (GST). n=4. FIG. 1C is a schematic diagram to show PIGF₁₂₃₋₁₄₁ added to the C-terminus of IL-1Ra and PDGF-BB to generate IL-1Ra/PIGF₁₂₃₋₁₄₁ and PDGF-BB/PIGF₁₂₃₋₁₄₁. PDGF-BB is naturally a dimer. FIG. 1D is a schematic representation of the ECM-mimetic hydrogel system and skin endogenous ECM. Fg, fibrinogen; Fn, fibronectin; Vn, vitronectin; TnC, tenascin C; HS, heparan sulfate. FIG. 1E provides graphs which show the cumulative release of IL-1Ra or PDGF-BB variants. n=4 in ECM-mimetic hydrogels generated with IL-1Ra or PDGF-BB variants and incubated in 10 times volume of buffer (containing or not plasmin) that was changed every 24 h. FIG. 1F shows the percentage of IL-1Ra and PDGF-BB variants remaining at the injection site (from a 5 mm diameter full thickness skin biopsy) was measured at various time points. n=4 per time point when IL-1Ra and PDGF-BB variants were injected intradermally in mice. For panels B, E, and F, data are means±SEM. For B, one-way ANOVA with Bonferroni post hoc test for pair-wise comparisons. For E and F, two-ways ANOVA with Bonferroni post hoc test for pair-wise comparisons. *P≤0.05, **P<0.01, ***P≤0.001.

FIG. 2 illustrates the binding-affinity of PIGF₁₂₃₋₁₄₁-fused IL-1Ra and PDGF-BB for ECM proteins. ELISA plate wells were coated with ECM proteins and further incubated with PIGF₁₂₃₋₁₄₁-fused or wild-type proteins. Graphs show signals given by an antibody detecting IL-1Ra or PDGF-BB. The signals were fitted by non-linear regression to obtain the dissociation constant (K_{d}) using A450 nm=Bmax*[protein]/(K_{d}+[protein]) where [protein] is the concentration of IL-1Ra, IL-1Ra/PIGF₁₂₃₋₁₄₁, PDGF-BB, or PDGF-BB/PIGF₁₂₃₋₁₄₁. Data are means±SEM. n=3.

FIG. 3 illustrates that fusing PIGF₁₂₃₋₁₄₁, to IL-1Ra and PDGF-BB does not impair their activity. 3A Bone marrow-derived macrophages were co-stimulated with IL-1β (1 ng/ml) and IL-1Ra variants at increasing concentrations (0 to 1 μg/ml). The negative control was no IL-1β treatment. The ability of IL-1Ra variants to inhibit IL-1β was assessed by measuring the release of IL-6 by macrophages 24 h after stimulation. n=4. 3B Dermal fibroblasts were cultured in basal media (2% serum) and stimulated with PDGF-BB variants at increasing concentration. The percentage of new cells was measured after 3 days. n=4. For both panels data are means±SEM and two-tailed Student's t-test. n.s.=non-significant.

FIG. 4 illustrates that IL-1R1 signalling impairs wound healing in diabetic mice. 4A Full-thickness wounds (5 mm) were created in diabetic mice (Lepr^{db/db}) and non-diabetic littermates (Lepr^{db/+}). Concentrations of IL-1β and IL-1Ra in harvested wounds at various time points. n=4 wounds per time point. 4(B) Concentrations of IL-1β in media of unstimulated or stimulated (LPS+ATP) bone marrow-derived macrophages from Lepr^{db/+} and Lepr^{db/db} mice. 4(C) Lepr^{db/db} mice were crossed with Il1r1^{-/-} mice to generate diabetic mice deficient for IL-1R1. Representative pictures of 14-week old mice are shown. Scale bar=1 cm. 4(D and E) Full-thickness wounds were created in Lepr^{db/db} and Lepr^{db/db}-Il1r1^{-/-} mice. Graph in D shows wound closure kinetics evaluated by histomorphometric analysis of tissue sections. n=12 wounds per time point. Representative histology (hematoxylin and eosin staining) after 10 d are shown in E. Black arrows indicate wound edges and gray arrows indicate tips of epithelium tongue. Scale bar=1 mm. For A, B, and C, data are means±SEM. For A and D, two-ways ANOVA with Bonferroni post hoc test for pair-wise comparisons. For B, two-tailed Student's t test. ***P≤0.001.

FIG. 5 shows the blood glucose levels of Lepr^{db/db} and Lepr^{db/db}_Il1r1 mice. Blood glucose of 14-week old mice were measured. n=4 mice per group. Data are means±SEM. One-way ANOVA with Bonferroni post hoc test for pair-wise comparisons. ***P<0.001, n.s.=non-significant.

FIG. 6 illustrates that super-affinity IL-1Ra promotes fast wound healing in diabetic mice. 6A and 6B—Full-thickness wounds in Lepr^{db/db} were treated with IL-1Ra or PDGF-BB variants (0.5 μg of wild-types, equimolar of engineered versions). Representative histology (hematoxylin and eosin staining) 9 d after treatment in 6A. Black arrows indicate wound edges and gray arrows indicate tips of epithelium tongue. Scale bar=1 mm. Wound closure evaluated by histomorphometric analysis of tissue sections in 6B. n=8 wounds per condition, except for PDGF-BB/PIGF₁₂₃₋₁₄₁ d 10 (n=10). 6C—Schematic representation of expected IL-1Ra/PIGF₁₂₃₋₁₄₁ effects in diabetic wounds. MMPs, matrix metallopeptidases; SASP, senescence-associated secretory phenotype; gray arrows represent mobilization; black arrow represent induction; red lines represent inhibition. 6D—Neutrophil and macrophage populations in wounds at various time points post-wounding measured by flow cytometry. Percentages are over total wound cells. Median fluorescence intensity (MFI) was measured for macrophages (F4/80+, CD11 b+ cells). n=8 wounds per time point. 6B and 6D, data are means±SEM. For 5B, two-ways ANOVA with Bonferroni post hoc test for pair-wise comparisons (for panel 6D, comparisons are done between saline and IL-1Ra/PIGF₁₂₃₋₁₄₁). *P≤0.05, **P≤0.001, ***P≤0.001, n.s.=non-significant.

FIG. 7 shows the gating strategy to analyse wound neutrophils and macrophages in wounds. Step by step representative flow cytometry dot plots are shown for the neutrophil and macrophage panels. MFI, median fluorescence intensity.

FIG. 8. shows that treatment with super-affinity IL-1Ra leads to a pro-healing microenvironment. 8A—Full-thickness wounds were created in Lepr^{db/db} mice and treated with saline or IL-1Ra variants (0.5 μg of wild-type, equimolar IL-1Ra/PIGF₁₂₃₋₁₄₁) and wound tissues were collected at various time points. Graphs show the concentration of cytokines, matrix metalloproteinases (MMPs), metallopeptidase inhibitor 1 (TIMP-1), and growth factors per ml of tissue lysate. n=4 per time point. 8B—Dermal fibroblasts were cultured with IL-1β (1 ng/ml) or saline control (PBS). After 9 d, SA-β-gal activity was assessed using senescence green probe (SGP). Graph shows percent increase in SGP signal. n=6. 8C—Full-thickness wounds in Lepr^{db/db} mice were treated with saline or IL-1Ra variants. After 9 d, SA-β-gal activity in wound fibroblast was assessed by flow cytometry using SGP. SA-β-gal activities were compared to fibroblasts from non-injured skin samples. For all panels, data are means±SEM. For 8A, two-ways ANOVA with Bonferroni post hoc test for pair-wise comparisons between saline and IL-1Ra/PIGF₁₂₃₋₁₄₁. For 8B, two-tailed Student's t test. For 8C, one-way ANOVA with Bonferroni post hoc test for pair-wise comparisons. * P≤0.05, ** P≤0.01, ***P≤0.001, n.s.=non-significant.

FIG. 9 shows the effect of IL-1β on senescence-associated secretory phenotype. Dermal fibroblasts were stimulated with IL-1β (1 ng/ml) for 24 h. The graphs show concentrations of senescence-associated cytokines measured in the media. n=4. For both panels, data are means±SEM. Two-tailed Student's t-test. ***P≤0.001.

FIG. 10 shows the gating strategy to analyse p-gal activity in wound fibroblasts. Step by step representative flow cytometry dot plots are shown. MFI, median fluorescence intensity.

FIG. 11 shows bone regeneration driven by BMP-2 and PDGF-BB is enhanced in Il1r1−/− mice. (11A and 11B) Critical-size calvarial defects (4.5 mm diameter) in wild-type or Il1r1−/− mice were treated with BMP-2 or PDGF-BB (1 μg) delivered by a fibrin matrix. Eight weeks after treatment, bone regeneration was measured by micro-computed tomography (microCT). Representative calvarial reconstructions are shown in 11A. Original defect area is shaded with a dashed outline. Coverage of defect and volume of new bone formed are shown in 11B. Data are means±SEM. n=6. Student's t-test. **P<0.01, ***P<0.001.

FIG. 12 shows surface-marker expression profiling of MSCs. MSCs were isolated from long compact bones of mice and expanded for 3 passages. Expression of MSC-specific surface markers was verified using flow cytometry. MSC phenotype was confirmed, since cells were CD11b−, CD19−, CD31−, CD45−, CD29+, CD44+, and CD90.2+, CD140b (PDGFR-b)+, and Sca-1 (Ly-6A/E)+. Signals of unstained cells are in white while signals of stained cells are in gray. Percentages of positive cells for the negative and positive markers are shown.

FIG. 13 shows IL-1β inhibits the morphogenic activity of BMP-2 and PDGF-BB. (13A and 13B) MSCs were cultured in growth medium or osteogenesis induction medium (OIM) containing BMP-2 and IL-1 β. Matrix mineralization was detected with alizarin red after 21 days. Representative wells (2 cm²) are shown in 13A. Expression of osteoblast-specific genes was determined by quantitative PCR after 7 and 14 days. Fold changes in gene expression relative to MSCs cultured in normal medium are shown in 13B. Alp, alkaline phosphatase; Runx2, runt-related transcription factor 2; Ibsp, integrin-binding sialoprotein. n=4 per time point. (13C and 13D) Primary MSCs were seeded for 10 days with PDGF-BB and IL-1 β. Representative wells (9 cm²) are shown in 13C. Graphs in 13D show colony-forming unit-fibroblasts (CFU-F) and average size of colonies. n=6 isolates. (13E) Proliferation of MSCs in low serum (1%, basal condition), upon stimulation with PDGF-BB and IL-1 β. Percentage increase in cell number over basal condition was measured after 72 h. n=6. (13F) MSC migration through transwell induced by PDGF-BB and IL-1 β. The number of cells per mm² that migrated after 6 h was counted and expressed as fold increase over basal migration. n=6. For panels 13B, 13D-F, data are means±SEM. For panel 13B, Student's t-test. For panels 13D-F, One-way ANOVA with Bonferroni post hoc test between PDGF-BB and PDGF-BB+IL-1 β. *P<0.05, **P<0.01, ***P<0.001. BMP-2, PDGF-BB, IL-1 β concentrations were respectively 50 ng/ml, 10 ng/ml, and 1 ng/ml.

FIG. 14 shows that IL-1β inhibits the activity of BMP-2 on osteoblasts. Osteoblasts isolated from calvarial bone were cultured in growth medium or osteogenesis induction medium (DIM) containing BMP-2 (10 ng/ml) with or without IL-1 β (1 ng/ml). After 7 and 14 days, expression of osteoblast-specific genes was determined by quantitative PCR. Fold changes in gene expression relative to MSCs cultured in normal medium are shown. Alpl, alkaline phosphatase; Runx2, runt-related transcription factor 2; lbsp, integrin-binding sialoprotein. Data are means±SEM (n=4). *P<0.05, **P<0.01, ***P<0.001; Student's t-test.

FIG. 15 shows that IL-1 β inhibits the activity of PDGF-BB on osteoblasts. (15A) Osteoblast proliferation in low serum (1%, basal condition) was stimulated with PDGF-BB (10 ng/ml) and IL-1 β (1 ng/ml). After 72 h, cell number increase over basal condition was measured. (15B) Osteoblast migration through transwell was induced by PDGF-BB (10 ng/ml) and IL-1β (1 ng/ml). After 6 h, the number of cells per square millimeter that passed through the transwell was counted and expressed as fold increase over basal migration. For panels a and b, data are means±SEM (n=6). One-way ANOVA with Bonferroni post hoc test; ***P<0.001.

FIG. 16 shows IL-1β makes cells less responsive to BMP-2 and PDGF-BB signalling. (16A-C) MSCs were cultured with IL-1β for 24 h. Smad1/5/8 levels were measured by ELISA in A. n=6. Smurf1 and Smurf2 expression was measured by quantitative PCR in 16B (fold change relative to 0 h). n=3. Smurf2 levels were determined by ELISA in 16C (concentration of Smurf2 per ml of cell lysate). n=4. (16D) MSCs were cultured in growth medium or osteogenesis induction medium (OIM) containing BMP-2, IL-1β, and heclin. Matrix mineralization was detected with alizarin red after 21 days. Representative wells (2 cm²) are shown. (16E) MSCs were cultured with IL-1β and heclin. Smad1/5/8 levels were measured by ELISA after 24 h. n=6. (16F) MSCs were stimulated with IL-1β for 4 h. Then, cells were stimulated with PDGF-BB (0 min time point). Phosphorylated Akt (S473, solid squares and circles, left y axis) and total Akt (open squares and circles, right y axis) per ml of cell lysate were measured by ELISA. n=4. (16G and 16H) MSCs were incubated with IL-1β. Phlpp1 and Phlpp2 expression was measured by quantitative PCR in 16G (fold change relative to 0 h). Graph in 16H shows PHLPP1 protein levels determined by ELISA after 24 h (per ml of cell lysate). For panels 16G and 16H. n=4. (16I) MSC proliferation in low serum (1%, basal condition) was stimulated with PDGF-BB, IL-1β, and NSC-45586. After 72 h, cell number increase over basal condition was measured. n=6. (16J) MSC migration through transwell was induced by PDGF-BB, IL-1β and NSC-45586. The number of migrated cells per mm² was counted and expressed as fold increase over basal migration. n=6. (16K) MSCs were incubated with NSC-45586 and IL-1β for 4 h. Then, cells were stimulated with PDGF-BB and phosphorylation of Akt (S473) was measured by ELISA after 120 min. Data are means±SEM. n=4. (16L and 16M) MSCs were cultured with IL-1β up to 14 days and SA-β-gal activity was assessed by measuring senescence green probe (SGP) with flow cytometry (representative dot plots in 16L and median fluorescence intensities in 16M). n=3. For all panels, data are means±SEM. For panels 16A-C, 16G, 16H and 16M, Student's t-test. For panel 16F, Two-way ANOVA with Bonferroni post hoc test. For panel 16E, 16I-K, One-way ANOVA with Bonferroni post hoc test. *P<0.05, **P<0.01, ***P<0.001, n.s.=non-significative. BMP-2, PDGF-BB, IL-1β concentrations were respectively 50 ng/ml, 10 ng/ml, and 1 ng/ml. Heclin and NSC-45586 concentrations were 20 mM.

FIG. 17 shows IL-1β makes osteoblasts less responsive to growth factor signalling. (A) Osteoblasts were cultured in basal medium with IL-1β (1 ng/ml). After 24 hours, Smad1/5/8 levels were measured by enzyme-linked immunosorbent assay (ELISA). Data are means±SEM (n=4); ***P<0.001; Student's t-test. (17B) Osteoblasts were incubated in low serum medium (1% FBS) supplemented with IL-1β (1 ng/ml) for 4 hours. Then, cells were stimulated with PDGF-BB (10 ng/ml). Phosphorylated Akt (S473) and total Akt were measured by ELISA after 6 h. Data are means±SEM (n=4). Student's t-test; n.s.=not significant, **P<0.01.

FIG. 18 shows MSCs treated with IL-18 release senescence-associated cytokines. MSCs were treated with PBS or IL-18 (5 ng/ml). After 48 h, cytokines released in the media were detected using an antibody array. Data are means±SEM (n=3 arrays). Student's t-test; ***P<0.001.

FIG. 19 shows delivering BMP-2 and PDGF-BB triggers IL-1β release by macrophages. (19A and 19B) Calvarial defects were treated with fibrin matrices containing saline, BMP-2 or PDGF-BB (1 mg). Fibrin matrices with bone tissue surrounding the defects were collected at different time points. Graph in 19A shows IL-1β concentration in the harvested sample detected by ELISA (IL-1β concentrations per ml of tissue lysate). n=3 per time point. Graph in 19B shows IL-1β concentrations measured at day 6 in mice with macrophages (control liposomes) or depleted of macrophages (clodronate liposomes). n=4. (19C) Primary macrophages were treated with BMP-2 or PDGF-BB. IL-1β concentration in the media after 24 h was detected by ELISA. n=4. Statistics are between no treatment (0 ng/ml) and treatments. For all panels, data are means±SEM. For panel 19A, Two-way ANOVA with Bonferroni post hoc test. For panel 19B, Student's t-test. For panel 19C, One-way ANOVA with Bonferroni post hoc test. *P<0.05, **P<0.01, ***P<0.001.

FIG. 20 shows clodronate liposomes deplete macrophages. Liposomes or control liposomes were injected in wild-type mice (7 mg/ml, 200 μl) 2 days prior to calvarial surgery. Additional 100 μl of clodronate liposomes or empty liposomes were injected right before surgery and every 2 days until day 6. The graphs show representative flow cytometry plots (20A) and the percentage of Ly6G⁻, F4/80⁺ collected from the spleen at day 6 (20B). About 80% of macrophages were depleted with clodronate liposomes. Data are means±SEM (n=4 mice). ***P<0.001; Student's t-test.

FIG. 21 shows macrophages express PDGF-BB and BMP-2 receptors. Bone marrow-derived primary macrophages were analyzed by flow cytometry for expression of PDGFRα, PDGFRβ and BMPR1A, BMPR2, ACVR1, and BMPR1B. Macrophages showed expression of PDGFRα, PDGFRβ and ACVR1, and to a lesser extent, of BMPR2 and BMPR1B, while BMPR1A was not detected. Signals from unstained cells are shown in white while signals from stained cells are gray. Percentages of positive cells for the receptors are indicated.

FIG. 22 illustrates the proposed mechanism by which IL-1R1 signalling desensitizes bone-forming cells to growth factors. IL-1R1 signalling in bone-forming cells (MSCs and osteoblasts) stimulates the expression of PHLPPs and Smurf2. Higher levels of PHLPPSs promote quicker dephosphorylation of Akt and dampen PDGF-BB signalling. Higher levels of Smurf2 impairs the responsiveness of cells to BMP-2 by promoting ubiquitination and thus degradation of Smad1/5/8. IL-1R1 signalling also accelerates senescence likely via Smurf2. BMP-2 and PDGF-BB further stimulate IL-1β release by macrophages. Phosphorylation is indicated by small circles with the letter P.

FIG. 23 shows BMP-2 strongly binds ubiquitous ECM proteins. ELISA plates were coated with an ECM protein (100 nM) and further incubated with BMP-2 at increasing concentration (0.1 to 100 nM). Bound BMP-2 was detected with an antibody. Specific-binding signals were fitted by non-linear regression to obtain the binding curves shown and the dissociation constant (K_{D}). K_{D} values are shown in each corresponding graph. Data are means±SEM (n=3).

FIG. 24 shows super-affinity IL-1Ra enhances the regenerative capacity of BMP-2 and PDGF-BB. (24A) Design of super-affinity PDGF-BB and IL-1Ra. The affinity for ECM proteins is high for BMP-2, medium for PDGF-BB and low for IL-1Ra. PIGF₁₂₃₋₁₄₁ (red oval) is added to the C-terminus of PDGF-BB and IL-1Ra to confer super-affinity for ECM proteins. (24B and 24C) Affinity (K_{D}) of wild-type versus PIGF₁₂₃₋₁₄₁-fused proteins for fibronectin (Fn), vitronectin (Vn), tenascin-C (Tnc) and fibrinogen (Fg) measured by ELISA. n=3. (24D) Fibrin matrices were made with growth factors and IL-1Ra variants and incubated in 10 times volume of buffer that was changed every day. The percentage of the recombinant proteins released from the matrices were quantified by ELISA. n=3. (24E and 24F) Critical-size calvarial defects (4.5 mm diameter) in wild-type mice were treated with growth factor (1 mg) and IL-1Ra (1 mg) variants delivered with fibrin. Eight weeks after treatment, regeneration was measured by microCT. Representative calvarial reconstructions are shown in 24E. Original defect area is shaded with a dashed outline. Coverage of defect and volume of new bone formed are shown in 24F. n=6. (24G and H) Critical-size femoral defects (2 mm wide, dashed boxes) in wild-type mice were treated with matrix-binding growth factors (1 mg) with or without super-affinity IL-1Ra (1 mg) delivered with fibrin. Twelve weeks after treatment, regeneration was measured by microCT. Representative femur reconstructions are shown in 24G. Quantification of the volume of new bone formed is shown in 24H. n=4. For all panels, data are means±SEM. For panels 24B, 24C, and 24H Student's t-test. For panel 24D, Two-way ANOVA with Bonferroni post hoc test. For panel 24F, One-way ANOVA with Bonferroni post hoc test for IL-1Ra only groups and PDGF-BB groups, Student's t-test for BMP-2 groups. *P<0.05, **P<0.01, ***P<0.001.

FIG. 25 provides various growth factor and IL-1Ra variant protein sequences.

FIG. 26 shows plasmin triggers the release of PIGF₁₂₃₋₁₄₁-fused proteins and BMP-2 from fibrin. Fibrin matrices containing growth factors or IL-1Ra variants were incubated in 10 times volume of release buffer containing plasmin that was changed daily. PDGF-BB and IL-1Ra are rapidly released. PDGF-BB/PIGF₁₂₃₋₁₄₁, IL-1Ra/PIGF₁₂₃₋₁₄₁ and BMP-2 are gradually released from the matrix over time. Data are means±SEM (n=3).

FIG. 27 shows that fusing PIGF₁₂₃₋₁₄₁ to PDGF-BB and IL-1Ra does not alter activity. (27A) Activity of PDGF-BB/PIGF₁₂₃₋₁₄₁ is similar to PDGF-BB in vitro. MSC proliferation in low serum (1%, basal condition) was stimulated with PDGF-BB or PDGF-BB/PIGF₁₂₃₋₁₄₁ at increasing concentrations. After 72 h, cell number increase over basal condition was measured. Data are means±SEM (n=6). One-way ANOVA with Bonferroni post hoc test between equal concentration; n.s.=not significant. (27B) Inhibitory activity of IL-1Ra/PIGF₁₂₃₋₁₄₁ is similar to IL-1Ra in vitro. MSC proliferation in low serum (1%, basal condition) was stimulated with PDGF-BB/PIGF₁₂₃₋₁₄₁ (20 ng/ml) in the presence of IL-1β (1 ng/ml) and IL-1Ra or IL-1Ra/PIGF₁₂₃₋₁₄₁ at increasing concentration. After 72 h, cell number increase over basal condition was measured. Data are means±SEM (n=6). One-way ANOVA with Bonferroni post hoc test between PDGF-BB/PIGF₁₂₃₋₁₄₁^{and} PDGF-BB/PIGF₁₂₃₋₁₄₁+IL-1β, and between equal concentrations of IL-1 Ra/PIGF₁₂₃₋₁₄₁; n.s.=not significant. ***P<0.001. (27C) MSCs were cultured in growth medium or osteogenesis induction medium (OIM) containing BMP-2 (50 ng/ml) in the presence of IL-1β (1 ng/ml) and IL-1Ra or IL-1Ra/PIGF₁₂₃₋₁₄₁ (100 ng/ml). After 21 days, matrix mineralization was revealed with alizarin red staining. Representative wells are shown (2 cm²).

FIG. 28 shows that delivering super-affinity IL-1Ra induces more M2-like macrophages. (28A) Critical-size calvarial defects (4.5 mm diameter) in wild-type mice were treated with a fibrin matrix. M2-like macrophages (CD11b⁺, F4/80⁺, CD206⁺) in the defect area were detected via flow cytometry after 3, 6 and 9 days. The percentage of anti-inflammatory macrophages gradually increase following bone injury. (28B) Critical-size calvarial defects (4.5 mm diameter) in wild-type mice were treated with a fibrin matrix containing IL-1Ra, IL-1Ra/PIGF₁₂₃₋₁₄₁ or saline control. The percentage of anti-inflammatory macrophages (CD11 b⁺, F4/80⁺, CD206⁺) in the defect area was detected via flow cytometry after 9 days. For both panels, data are means±SEM (n=5 per time point or condition). One-way ANOVA with Bonferroni post hoc test to compare saline versus IL-1 Ra variants; **P<0.01.

FIG. 29 shows the binding affinity of PIGF-2, NRTN and AREG for ECM proteins. PIGF-2 and AREG show a higher affinity for ECM proteins. The results obtained by ELISA assay (n=3, mean±SEM). The K_{D} values were measured by using a one site specific binding model: A450 nm=Bmax*[ECM protein]/(K_{D}+[ECM protein]). The K_{D} values are shown in Table 3.

FIG. 30 shows the release of AREG, PIGF-1, and PIGF-2 from a fibrin matrix. Over 60% PIGF-1 was released after two days while less than 30% of PIGF-2 and AREG were released after 7 days (n=3, mean±SEM).

FIG. 31 shows the binding affinity of PIGF-2, AREG, and NRTN fragments for ECM proteins and heparan sulphate. PIGF-2₁₂₃₋₁₄₁ (RRRPKGRGKRRREKQRPTD), NRTN₁₄₆₋₁₅₇ (RRLRQRRRLRRE) and AREG₁₂₆₋₁₃₈ (RKKKGGKNGKNRR) display a higher affinity for both ECM proteins and heparan sulphate. The results obtained by ELISA assay (n=3, mean±SEM). The summary of results is shown in Table 5.

FIG. 32 shows the binding-affinity of AREG₁₂₈₋₁₃₈/PDGF-BB and PDGF-BB for ECM proteins fibronectin, vitronectin, tenascin C, and fibrinogen.

FIG. 33 A shows representative histology (hematoxylin and eosin staining) 7 or 9 d post-treatment of full-thickness wounds in Lepr^{db/db} treated with PDGF-BB variants. FIG. 33 B shows wound closure following treatment with PDGF-BB variants evaluated by histomorphometric analysis of tissue sections.

FIG. 34 is a graph showing the surface electrical capacitance given in dermal phase meter arbitrary units to illustrate the epithelial barrier properties of IL-1Ra/PIGF₁₂₃₋₁₄₁-treated wounds.

FIG. 35 A shows wound closure in non-diabetic mice 6 days following treatment with saline or IL-1Ra PIGF₁₂₃₋₁₄₁, evaluated by histomorphometric analysis of tissue sections. FIG. 35 B provides representative histology (hematoxylin and eosin staining) 7 or 9 d post treatment of full-thickness wounds in wild-type (C57BL/6) mice treated with saline or IL-1 Ra/PIGF₁₂₃₋₁₄₁.

### DETAILED DESCRIPTION

As used in this specification and the appended claims, the singular forms “a”, “an” and “the” include plural referents unless the context clearly dictates otherwise. The terms “a” (or “an”), as well as the terms “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Unless defined otherwise, all technical and scientific terms used herein have the same meaning as commonly understood by one of ordinary skill in the art to which this invention is related.

Units, prefixes, and symbols are denoted in their Système International d'Unités (SI) accepted form. Numeric ranges are inclusive of the numbers defining the range. Unless otherwise indicated, amino acid sequences are written left to right in amino to carboxy orientation. The headings provided herein are not limitations of the various aspects or embodiments of the invention, which can be had by reference to the specification as a whole. Accordingly, the terms defined immediately below are more fully defined by reference to the specification in its entirety.

It is understood that wherever embodiments are described herein with the language “comprising,” otherwise analogous embodiments described in terms of “consisting of” and/or “consisting essentially of” are also provided.

Amino acids are referred to herein by either their commonly known three letter symbols or by the one-letter symbols recommended by the IUPAC-IUB Biochemical Nomenclature Commission. Nucleotides, likewise, are referred to by their commonly accepted single-letter codes.

As used herein, a “fusion protein” is a protein made from a fusion gene, which is created by joining of two or more genes that originally coded for separate polypeptides.

As used herein “polypeptide” refers to any sequence of two or more amino acids, regardless of length, post-translation modification, or function. Polypeptides can include natural amino acids and non-natural amino acids. Polypeptides can also be modified in any of a variety of standard chemical ways (e.g., an amino acid can be modified with a protecting group; the carboxy-terminal amino acid can be made into a terminal amide group; the amino-terminal residue can be modified with groups to, e.g., enhance lipophilicity; or the polypeptide can be chemically glycosylated or otherwise modified to increase stability or in vivo half-life). Polypeptide modifications can include the attachment of another structure such as a cyclic compound or other molecule to the polypeptide and can also include polypeptides that contain one or more amino acids in an altered configuration (i.e., R or S; or, L or D).

As used herein “peptide” means any chain of amino acids from 12 to 50 amino acid residues in length, preferably 12 to 40, 12 to 30, 12 to 25, or 12 to 24, or more preferably about 12, 13, 14, 15, 16, 17, 18, 19, 20, 21, 22, 23 or 24 amino acid residues in length.

An illustrative sequence for Interleukin-1 receptor antagonist is provided as SEQ ID NO:4.

Human Interleukin-1 receptor antagonist (IL-1Ra)

(SEQ ID NO: 4)
RPSGRKSSKMQAFRIWDVNQKTFYLRNNQLVAGYLQGPNVNLEEKIDVVP
IEPHALFLGIHGGKMCLSCVKSGDETRLQLEAVNITDLSENRKQDKRFAF
IRSDSGPTTSFESAACPGWFLCTAMEADQPVSLTNMPDEGVMVTKFYFQE
DE

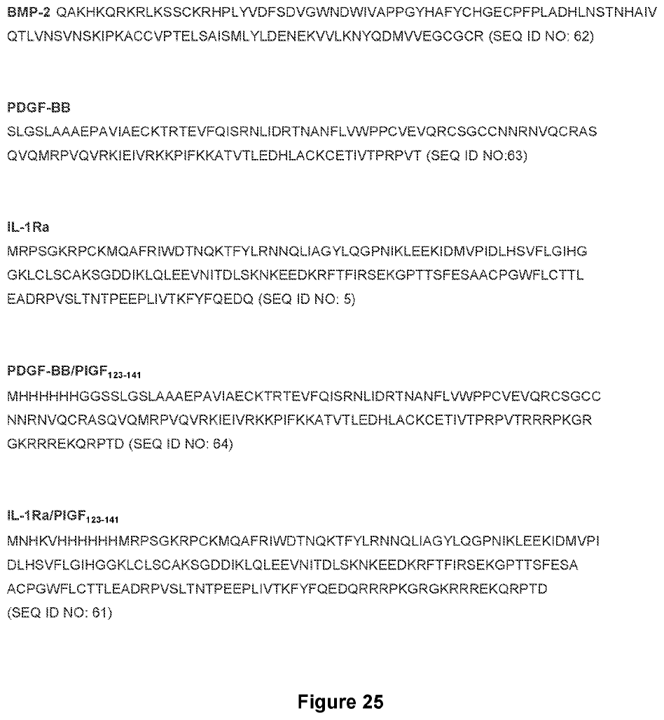

A further illustrative sequence for Interleukin-1 receptor antagonist is provided in FIG. 25 (SEQ ID NO: 5—mouse IL-1Ra).

The extracellular matrix (ECM) provides structural support for tissue and signalling capabilities for cells. The ECM plays an important role in development and tissue repair. The invention provides fusion proteins comprising IL-1Ra and peptides that specifically bind ECM proteins.

Specific binding, as that term is commonly used in the biological arts, refers to a molecule that binds to a target with a relatively high affinity compared to non-target tissues, and generally involves a plurality of non-covalent interactions, such as electrostatic interactions, van der Waals interactions, hydrogen bonding, and the like. Specific binding interactions characterize antibody-antigen binding, enzyme-substrate binding, and specifically binding protein-receptor interactions; while such molecules may bind tissues besides their targets from time to time, such binding is said to lack specificity and is not specific binding.

As used herein, a peptide that specifically binds to one or more or all extracellular matrix proteins selected from the group consisting of fibrinogen, fibronectin, vitronectin, tenascin C and heparan sulfate in preference to other proteins. In one embodiment a peptide that specifically binds to one or more or all ECM proteins binds with high affinity, preferably with a dissociation constant (K_{D}) of less than about 300 nM, or less than about 200 nM, or less than about 100 nM, or less than about 40 nM, or less than about 25 nM or less than about 15 nM or less than about 10 nM.

PIGF is an angiogenic cytokine that exists in multiple splice variants. PIGF was originally identified in the placenta, where it has been proposed to control trophoblast growth and differentiation. PIGF is expressed during early embryonic development. PIGF has been shown to be expressed in the villous trophoblast, while vascular endothelial growth factor (VEGF) is expressed in cells of mesenchymal origin within the chorionic plate. PIGF is expressed in several other organs including the heart, lung, thyroid, skeletal muscle, and adipose tissue. PIGF acts as a potent stimulator of VEGF secretion by monocytes and significantly increases mRNA levels of the proinflammatory chemokines interleukin-1 beta, interleukin-8, monocyte chemoattractant protein-1 and VEGF in peripheral blood mononuclear cells of healthy subjects. PIGF induces tumor angiogenesis by recruiting circulating hematopoietic progenitor cells and macrophages to the site of the growing tumors.

Illustrative sequences for PIGF variants comprising an ECM binding peptide (underlined) are provided below.

Placenta growth factor-2 (PIGF-2):

(SEQ ID NO: 6)
MPVMRLFPCFLQLLAGLALPAVPPQQWALSAGNGSSEVEVVPFQEVWGRS
YCRALERLVDVVSEYPSEVEHMFSPSCVSLLRCTGCCGDENLHCVPVETA
NVTMQLLKIRSGDRPSYVELTFSQHVRCECRPLREKMKPERRRPKGRGKR
RREKQRPTDCHLCGDAVPRR

Placenta growth factor-4 (PIGF-4):

(SEQ ID NO: 7)
MPVMRLFPCFLQLLAGLALPAVPPQQWALSAGNGSSEVEVVPFQEVWGRS
YCRALERLVDVVSEYPSEVEHMFSPSCVSLLRCTGCCGDENLHCVPVETA
NVTMQLLKIRSGDRPSYVELTFSQHVRCECRHSPGRQSPDMPGDFRADAP
SFLPPRRSLPMLFRMEWGCALTGSQSAVWPSSPVPEEIPRMHPGRNGKKQ
QRKPLREKMKPERRRPKGRGKRRREKQRPTDCHLCGDAVPRR

The heparin binding domain of PIGF-2 is located at PIGF₁₂₃₋₁₅₂,

SEQ ID NO: 8
RRRPKGRGKRRREKQRPTDCHLCGDAVPRR,.

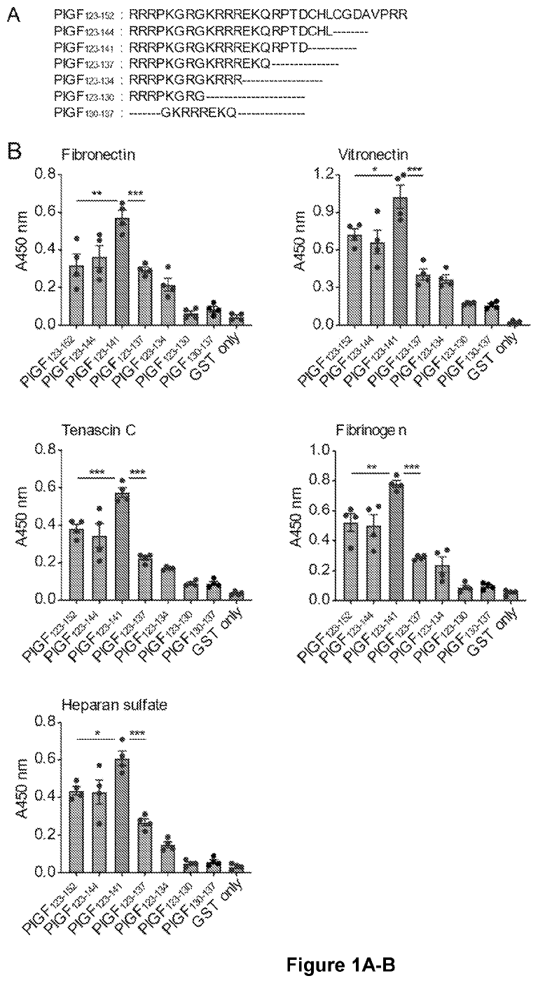

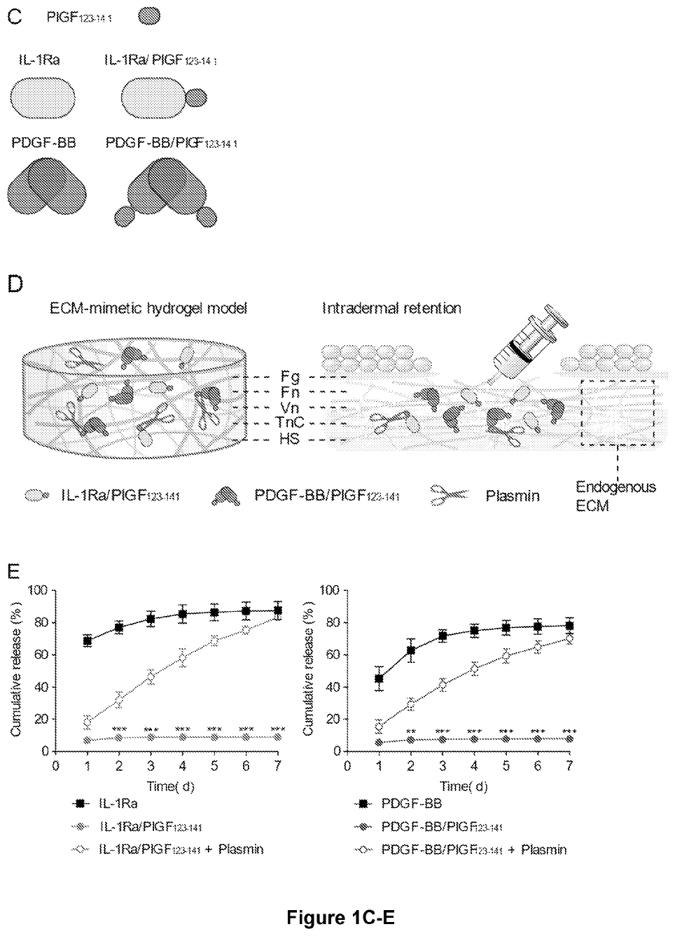

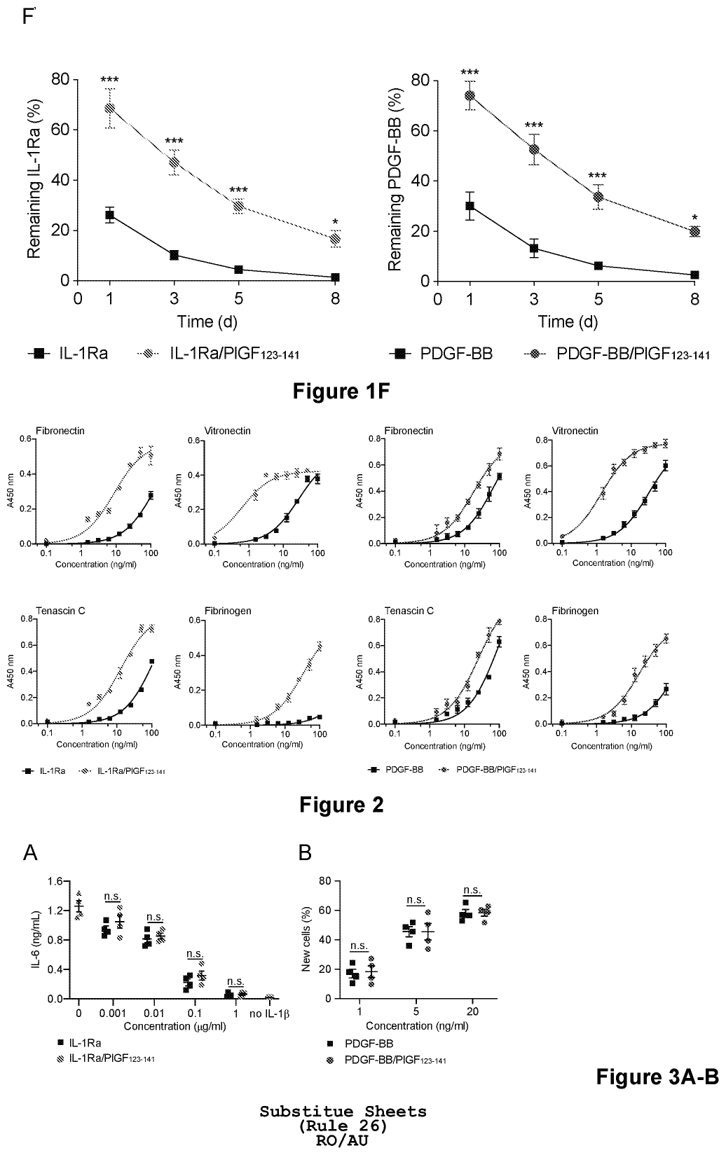

As shown in FIG. 1(B) SEQ ID NO: 1 binds very strongly to fibrinogen, as well as the extracellular matrix proteins fibronectin, vitronectin and tenascin C. SEQ ID NO:1 is referred to as PIGF₁₂₃₋₁₄₁.

Fragment and variants of SEQ ID NO: 8 comprising SEQ ID NO: 1 may be used in the fusion protein of the first aspect. These include orthologues of SEQ ID No: 1.
RRKTKGKRKRSRNSQTEE, SEQ ID NO:9—the mouse equivalent of SEQ ID NO:1;
RRRPKGRGKRKREKQRPTD, SEQ ID NO: 10—placenta growth factor isoform X7 [Canis lupus familiaris]
RRRPKGRGKRRREKQKPTD, SEQ ID NO: 11—placenta growth factor isoform X1 [Hylobates moloch]
RRRPKGRGKRRREKQRPKD, SEQ ID NO: 12—growth factor isoform X1 [Callithrix jacchus]
RRRPKGRGKRKRDKQRPTD, SEQ ID NO: 13—placenta growth factor isoform X1 [Galeopterus variegatus]
RRRPKGRGKRKREKQKPTD, SEQ ID NO: 14—placenta growth factor isoform X1 [Carlito syrichta]
RRRPKGRGKRKREKQRHTD, SEQ ID NO: 15—placenta growth factor isoform X3 [Lagenorhynchus obliquidens]
RRRPKGRGKRKREKQRPRD, SEQ ID NO: 16—placenta growth factor isoform X3 [Suricata suricatta]
RRRSRGRGKRKREKQRPTD, SEQ ID NO: 17—placenta growth factor isoform X1 [Trichechus manatus latirostris]
RRRPKGQGKRRREKQRP, SEQ ID NO: 18—placenta growth factor isoform X1 [Pelodiscus sinensis]
RRRPKGSGKRKKEKQRPTD, SEQ ID NO: 19—placenta growth factor isoform X1 [Eptesicus fuscus]
RRRPKGQGKRKREKQRP, SEQ ID NO: 20—placenta growth factor isoform X1 [Chrysemys picta bellii]
RRRHKGRRKRKREKQRPTD, SEQ ID NO: 21—placenta growth factor isoform X1 [Ailuropoda melanoleuca]
RRRPKVRGKRKREKQKPT, SEQ ID NO: 22—placenta growth factor isoform X1 [Fukomys damarensis]
RRRPKGRSKRKRAKQRPKD, SEQ ID NO: 23—placenta growth factor [Podarcis muralis]
RRRPKGRSKRKRAKQRPKD, SEQ ID NO: 24—placenta growth factor [Lacerta agilis]
RRRLKGRGKRKKEKQRSTD, SEQ ID NO: 25—placenta growth factor isoform X1 [Ictidomys tridecemlineatus]
RRRPKVRGKRKRENQKPT, SEQ ID NO: 26—placenta growth factor isoform X1 [Cavia porcellus]
RRRYRGRGKRKREKQRATD, SEQ ID NO: 27—placenta growth factor [Echinops telfairi]
RRRPKGRGRKRKEKQR, SEQ ID NO: 28—placenta growth factor-like [Electrophorus electricus]
RRRNKGSGKRKKEKQRPT, SEQ ID NO: 29—placenta growth factor [Phyllostomus discolor]
RRRPKGRGKRRGEKKRRKD, SEQ ID NO: 30—placenta growth factor [Anas platyrhynchos]
RRRQKGRGRKRKDKQRPKD, SEQ ID NO: 31—placenta growth factor [Paramormyrops kingsleyae]
RRRPKGRGKRRQDKMR, SEQ ID NO: 32—placenta growth factor [Strigops habroptila]
RRRSKDRGKRKRERPRPT, SEQ ID NO: 33—placenta growth factor [Ornithorhynchus anatinus]
RRRPKGRGKRRQERTR, SEQ ID NO: 34—placenta growth factor [Amazona aestiva]
RRRPRGRGRKRKEKQR, SEQ ID NO: 35—PREDICTED: vascular endothelial growth factor A-like [Pygocentrus nattereri]
RRRFKGRGKRKRDKQRTKD, SEQ ID NO: 36—placenta growth factor [Sarcophilus harrisii]
RRRPKGRGKRKREKQRPTD, SEQ ID NO: 37—consensus placental growth factor sequence (differs by only one amino acid from the human sequence)

Additional ECM binding peptides from PIGF include the following amino acid sequences or conservative variations thereof.

SEQ ID NO: 38
RRRPKGRGKRRREKQRPTDC,
PIGF₁₂₃₋₁₄₂
SEQ ID NO: 39
RRRPKGRGKRRREKQRPTDCH,
PIGF₁₂₃₋₁₄₃
SEQ ID NO: 40
RRRPKGRGKRRREKQRPTDCHLC,
PIGF₁₂₃₋₁₄₅
SEQ ID NO: 41
RRRPKGRGKRRREKQRPTDCHLCG,
PIGF₁₂₃₋₁₄₆
SEQ ID NO: 42
RRRPKGRGKRRREKQRPTDCHLCGD,
PIGF₁₂₃₋₁₄₇
SEQ ID NO: 43
RRRPKGRGKRRREKQRPTDCHLCGDA,
PIGF₁₂₃₋₁₄₈
SEQ ID NO: 44
RRRPKGRGKRRREKQRPTDCHLCGDAV,
PIGF₁₂₃₋₁₄₉
SEQ ID NO: 45
RRRPKGRGKRRREKQRPTDCHLCGDAVP,
PIGF₁₂₃₋₁₅₀
SEQ ID NO: 46
RRRPKGRGKRRREKQRPTDCHLCGDAVPR,
PIGF₁₂₃₋₁₅₁

Additional ECM binding peptides from amphiregulin (AREG) include the following amino acid sequences or conservative variations thereof.

SEQ ID NO: 47
RKKKGGKNGKNRRN
AREG₁₂₆₋₁₃₉
SEQ ID NO: 48
RKKKGGKNGKNRRNR
AREG₁₂₆₋₁₄₀
SEQ ID NO: 49
RKKKGGKNGKNRRNRK
AREG₁₂₆₋₁₄₁
SEQ ID NO: 50
RKKKGGKNGKNRRNRKK
AREG₁₂₆₋₁₄₂
SEQ ID NO: 51
RKKKGGKNGKNRRNRKKK
AREG₁₂₆₋₁₄₃
SEQ ID NO: 52
RKKKGGKNGKNRRNRKKKN
AREG₁₂₆₋₁₄₄
SEQ ID NO: 53
RKKKGGKNGKNRRNRKKKNP
AREG₁₂₆₋₁₄₅
SEQ ID NO: 54
RKKKGGKNGKNRRNRKKKNPC
AREG₁₂₆₋₁₄₆
SEQ ID NO: 55
RKKKGGKNGKNRRNRKKKNPCN
AREG₁₂₆₋₁₄₇
SEQ ID NO: 56
RKKKGGKNGKNRRNRKKKNPCNA
AREG₁₂₆₋₁₄₈
SEQ ID NO: 57
KPKRKKKGGKNGKNRRNRKKKNP
AREG₁₂₃₋₁₄₅
SEQ ID NO: 58
PKRKKKGGKNGKNRRNRKKKNP
AREG₁₂₄₋₁₄₅
SEQ ID NO: 59
KRKKKGGKNGKNRRNRKKKNP
AREG₁₂₅₋₁₄₅

The present invention also extends to fusion proteins comprising orthologues, functional homologues or variants of IL-1Ra which are capable of antagonizing IL-1R and/or peptides which are orthologues, functional homologues or variants of the ECM binding peptide.

An orthologue as used herein is the equivalent of the protein or peptide used in the fusion protein of the first aspect whose sequence is derived from a non-human animal, preferably a mammal.

Functional homologues or variants may be derived by insertion, deletion or substitution of amino acids in, or chemical modification of, the native carboxyl-terminal sequence. Amino acid insertion variants include amino and/or carboxylic terminal fusions as well as intra-sequence insertions of single or multiple amino acids. Insertion amino acid sequence variants are those in which one or more amino acid residues are introduced into a predetermined site in the protein although random insertion is also possible with suitable screening of the resulting product. Deletion variants are characterised by the removal of one or more amino acids from the sequence. Substitution amino acid variants are those in which at least one amino acid residue in the sequence has been replaced by another of the twenty, primary protein amino acids, or by a non-protein amino acid. In one embodiment substitutions are with conservative amino acids.

A conservative substitution is where one amino acid residue is substituted by another with similar biochemical properties, e.g. charge, hydrophobicity and size.

Conservative substitutions include those between the following classes of amino acid residues:
Aliphatic—G, A, L, I, V
Hydroxyl or sulfur containing—S, C, T, M
Aromatic—F, Y, W
Basic—R, K, H
Acidic and their amides—D, E, N, Q

In one embodiment variants of IL-1Ra or the ECM binding peptide used in the fusion protein of the invention may comprise one, two, three, four or five insertions, deletions or substitutions compared to the natural IL-1Ra or ECM binding peptide, provided that the function of the native sequence is retained.

In one embodiment amino acids, except for glycine, are of the L-absolute configuration. D configuration amino acids may also be used.

Persons skilled in the art will appreciate that the IL-1Ra or ECM binding peptide used may be modified to improve storage stability, bioactivity, circulating half-life, or for any other purpose using methods available in the art, such as glycosylation, by conjugation to a polymer to increase circulating half-life, by pegylation or other chemical modification.

Variants of the human IL-1Ra sequence provided as SEQ ID NO: 4 or the ECM binding peptides of SEQ ID NOs: 1, 2 or 3 preferably have at least about 80% amino acid sequence identity with the relevant human sequence as disclosed herein (the reference sequence). Ordinarily, a variant will have at least about 80% amino acid sequence identity, alternatively at least about 81%, 82%, 83%, 84%, 85%, 86%, 87%, 88%, 89%, 90%, 91%, 92%, 93%, 94%, 95%, 96%, 97%, 98%, or 99% amino acid sequence identity, to the reference sequence.

In some cases, a determination of the percent identity of a peptide or protein to a sequence set forth herein may be required. In such cases, the percent identity is measured in terms of the number of residues of the peptide or protein, or a portion of the peptide or protein. A polypeptide of, e.g., 90% identity, may also be a portion of a larger polypeptide or protein.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

In the fusion protein of the invention, the ECM binding peptide is inserted at or near the C-terminus or N-terminus of IL-1Ra. Insertion of the ECM binding peptide at the C-terminus or N-terminus may change the stability of the fusion protein.

In the fusion proteins of the invention, the IL-1Ra may be directly linked to the ECM binding peptide or indirectly linked by a linker. In an embodiment a linker is present between the IL-1Ra and ECM binding peptide. Suitable linkers comprise Glycine and Serine, for example GGS or SGG or repeats thereof.

Preferably the linker sequence comprises from about 1 to 20 amino acids, more preferably from about 1 to 16 amino acids. The linker sequence is preferably flexible so as not hold the IL-1Ra in a single undesired conformation.

The fusion protein as described herein may additionally comprise an N-terminal signal peptide domain, which allows processing, e.g., extracellular secretion, in a suitable host cell. Preferably, the N-terminal signal peptide domain comprises a protease, e.g., a signal peptidase cleavage site and thus may be removed after or during expression to obtain the mature protein.

Further, the fusion protein may comprise comprises a recognition/purification domain, e.g., a Strep-tag domain and/or a poly-His domain, which may be located at the N-terminus or at the C-terminus. In one embodiment the fusion protein comprises a histidine tag at the N-terminus.

In a particular embodiment, the fusion protein comprises IL-1Ra of SEQ ID NO: 2 or a variant thereof having at least 80% sequence identify thereto, linked to an ECM binding peptide of SEQ ID NO: 1, SEQ ID NO: 2 or SEQ ID NO: 3 or any one of SEQ ID NO: 8-59, either directly or via a linker, preferably comprising GGS or SGG or repeats thereof. The ECM binding peptide may be at the N or C terminus of the fusion protein.

In a particular embodiment the fusion protein comprises any one of SEQ ID NO: 1, 2 or 3 or 8-59 or a variant thereof having at least 80% sequence identify thereto, linked at its C terminus to SEQ ID NO: 4 or SEQ ID NO: 5, optionally via a linker such as SGG or GGS or SGGSGG or GGSGGS.

In a particular embodiment the fusion protein comprises any one of SEQ ID NO: 1, 2 or 3 or 8-59 or a variant thereof having at least 80% sequence identify thereto, linked at its N terminus to SEQ ID NO: 4 or SEQ ID NO: 5, optionally via a linker such as SGG or GGS or SGGSGG or GGSGGS.

In a particular embodiment the fusion protein comprises SEQ ID NO: 1 or a variant thereof having at least 80% sequence identify thereto, linked at its C terminus to SEQ ID NO: 4, optionally via a linker such as SGG or GGS or SGGSGG or GGSGGS.

In a particular embodiment the fusion protein comprises SEQ ID NO: 1 or a variant thereof having at least 80% sequence identify thereto, linked at its N terminus to SEQ ID NO: 4, optionally via a linker such as SGG or GGS or SGGSGG or GGSGGS.

In a preferred embodiment the fusion protein comprises SEQ ID NO: 4 (human IL1-Ra) with PIGF₁₂₃₋₁₄₁ (SEQ ID NO: 1—bold) at its C terminus, to provide IL1-Ra/PIGF₁₂₃₋₁₄₁ with the following amino acid sequence:

(SEQ ID NO: 60)
RPSGRKSSKMQAFRIWDVNQKTFYLRNNQLVAGYLQGPNVNLEEKIDVVP
IEPHALFLGIHGGKMCLSCVKSGDETRLQLEAVNITDLSENRKQDKRFAF
IRSDSGPTTSFESAACPGWFLCTAMEADQPVSLTNMPDEGVMVTKFYFQE
DE RRRPKGRGKRRREKQRPTD.

In a preferred embodiment the fusion protein comprises SEQ ID NO: 5 (mouse IL1-Ra) with PIGF₁₂₃₋₁₄₁ (SEQ ID NO: 1—bold) at its C terminus and a histidine tag (underlined) at the N terminus, to provide IL1-Ra/PIGF₁₂₃₋₁₄₁ with the following amino acid sequence as shown in FIG. 25 (SEQ ID NO: 61):

(SEQ ID NO: 61)
MNHKVHHHHHHMRPSGKRPCKMQAFRIWDTNQKTFYLRNNQLIAGYLQGP
NIKLEEKIDMVPIDLHSVFLGIHGGKLCLSCAKSGDDIKLQLEEVNITDL
SKNKEEDKRFTFIRSEKGPTTSFESAACPGWFLCTTLEADRPVSLTNTPE
EPLIVTKFYFQEDQRRRPKGRGKRRREKQRPTD.

In general, preparation of the fusion proteins of the invention can be accomplished by procedures disclosed herein and by recognized recombinant DNA techniques involving, e.g., polymerase chain amplification reactions (PCR), preparation of plasmid DNA, cleavage of DNA with restriction enzymes, preparation of oligonucleotides, ligation of DNA, isolation of mRNA, introduction of the DNA into a suitable cell, transformation or transfection of a host, culturing of the host. Additionally, the fusion proteins can be isolated and purified using chaotropic agents and well known electrophoretic, centrifugation and chromatographic methods.

The invention further provides nucleic acid sequences and particularly DNA sequences that encode the present fusion proteins. Preferably, the DNA sequence is carried by a vector suited for extrachromosomal replication such as a phage, virus, plasmid, phagemid, cosmid, YAC, or episome. In particular, a DNA vector that encodes a desired fusion protein can be used to facilitate preparative methods described herein and to obtain significant quantities of the fusion protein. The DNA sequence can be inserted into an appropriate expression vector, i.e., a vector which contains the necessary elements for the transcription and translation of the inserted protein-coding sequence. A variety of host-vector systems may be utilized to express the protein-coding sequence. These include mammalian cell systems infected with virus (e.g., vaccinia virus, adenovirus, etc.); insect cell systems infected with virus (e.g., baculovirus); microorganisms such as yeast containing yeast vectors, or bacteria transformed with bacteriophage DNA, plasmid DNA or cosmid DNA. Depending on the host-vector system utilized, any one of a number of suitable transcription and translation elements may be used.

In general, a preferred DNA vector according to the invention comprises a nucleotide sequence linked by phosphodiester bonds comprising, in a 5′ to 3′ direction a first cloning site for introduction of a first nucleotide sequence encoding II-1 RA operably linked to a nucleotide sequence encoding an ECM binding peptide.

In most instances, it will be preferred that each of the fusion protein components encoded by the DNA vector be provided in a “cassette” format. By the term “cassette” is meant that each component can be readily substituted for another component by standard recombinant methods.

The fusion proteins described herein are preferably produced by standard recombinant DNA techniques. The resultant hybrid DNA molecule can be expressed in a suitable host cell to produce the fusion protein. The DNA molecules are ligated to each other in a 5′ to 3′ orientation such that, after ligation, the translational frame of the encoded polypeptides is not altered (i.e., the DNA molecules are ligated to each other in-frame). The resulting DNA molecules encode an in-frame fusion protein.

The components of the fusion protein can be organized in nearly any order provided each is capable of performing its intended function.

A number of strategies can be employed to express the fusion proteins of the invention. For example, the gene fusion construct described above can be incorporated into a suitable vector by known means such as by use of restriction enzymes to make cuts in the vector for insertion of the construct followed by ligation. The vector containing the gene construct is then introduced into a suitable host for expression of the fusion protein. Selection of suitable vectors can be made empirically based on factors relating to the cloning protocol. For example, the vector should be compatible with, and have the proper replicon for the host that is being employed. Further the vector must be able to accommodate the DNA sequence coding for the fusion protein that is to be expressed. Suitable host cells include eukaryotic and prokaryotic cells, preferably those cells that can be easily transformed and exhibit rapid growth in culture medium. Specifically, preferred hosts cells include prokaryotes such as E. coli, Bacillus subtillus, etc. and eukaryotes such as animal cells and yeast strains, e.g., S. cerevisiae. Mammalian cells are generally preferred, particularly J558, NSO, SP2-O or CHO. Other suitable hosts include, e.g., insect cells such as Sf9. Conventional culturing conditions are employed. Stable transformed or transfected cell lines can then be selected. Cells expressing fusion proteins according to the invention can be determined by known procedures.

Nucleic acid encoding a desired fusion protein can be introduced into a host cell by standard techniques for transfecting cells. The term “transfecting” or “transfection” is intended to encompass all conventional techniques for introducing nucleic acid into host cells, including calcium phosphate co-precipitation, DEAE-dextran-mediated transfection, lipofection, electroporation, microinjection, viral transduction and/or integration.

The present invention further provides a production process for isolating a fusion protein of interest. In the process, a host cell (e.g., a yeast, fungus, insect, bacterial or animal cell), into which has been introduced a nucleic acid encoding the fusion protein operatively linked to a regulatory sequence, is grown at production scale in a culture medium. Subsequently, the fusion protein of interest is isolated from harvested host cells or from the culture medium. Standard protein purification techniques can be used to isolate the fusion protein from the medium or from the harvested cells. In particular, the purification techniques can be used to express and purify a desired fusion protein on a large-scale (i.e. in at least milligram quantities) from a variety of implementations including roller bottles, spinner flasks, tissue culture plates, bioreactor, or a fermentor.

An expressed fusion protein can be isolated and purified by known methods. Typically, the culture medium is centrifuged and then the supernatant is purified by affinity or immunoaffinity chromatography, e.g. Protein-A or Protein-G affinity chromatography or an immunoaffinity protocol comprising use of monoclonal antibodies that bind the expressed fusion protein. The fusion proteins of the present invention can be separated and purified by appropriate combination of known techniques. These methods include, for example, methods utilizing solubility such as salt precipitation and solvent precipitation, methods utilizing the difference in molecular weight such as dialysis, ultra-filtration, gel-filtration, and SDS-polyacrylamide gel electrophoresis, methods utilizing a difference in electrical charge such as ion-exchange column chromatography, methods utilizing specific affinity such as affinity chromatograph, methods utilizing a difference in hydrophobicity such as reverse-phase high performance liquid chromatograph and methods utilizing a difference in isoelectric point, such as isoelectric focusing electrophoresis, metal affinity columns such as Ni-NTA.

It is preferred that the fusion proteins of the present invention be substantially pure. That is, the fusion proteins have been isolated from cell substituents that naturally accompany it so that the fusion proteins are present preferably in at least 80% or 90% to 95% homogeneity (w/w). Fusion proteins having at least 98 to 99% homogeneity (w/w) are most preferred for many pharmaceutical, clinical and research applications. Once substantially purified the fusion protein should be substantially free of contaminants for therapeutic applications. Once purified partially or to substantial purity, the soluble fusion proteins can be used therapeutically, or in performing in vitro or in vivo assays as disclosed herein. Substantial purity can be determined by a variety of standard techniques such as chromatography and gel electrophoresis.

Fusion proteins according to the invention may be administered in a pharmaceutical composition optionally together with pharmaceutically acceptable carriers or excipients for administration. Fusion proteins according to the invention may be administered in a veterinary composition optionally together with carriers or excipients suitable for administration to animals.

The pharmaceutical diluents, excipients, extenders, or carriers (termed herein as a pharmaceutically acceptable carrier, or a carrier) are suitably selected with respect to the intended form of administration and as consistent with conventional pharmaceutical practices.

Pharmaceutically acceptable carriers or excipients may be used to deliver embodiments as described herein. Excipient refers to an inert substance used as a diluent or vehicle for a therapeutic agent. Pharmaceutically acceptable carriers are used, in general, with a compound so as to make the compound useful for a therapy or as a product. In general, for any substance, a carrier is a material that is combined with the substance for delivery to an animal. Conventional pharmaceutical carriers, aqueous, powder or oily bases, thickeners and the like may be necessary or desirable. In some cases, the carrier is essential for delivery, e.g., to solubilize an insoluble compound for liquid delivery; a buffer for control of the pH of the substance to preserve its activity; or a diluent to prevent loss of the substance in the storage vessel. In other cases, however, the carrier is for convenience, e.g., a liquid for more convenient administration. Pharmaceutically acceptable salts of the compounds described herein may be synthesized according to methods known to those skilled in the arts. Pharmaceutically acceptable substances or compositions are highly purified to be free of contaminants, are sterile, and are biocompatible. They further may include a carrier, salt, or excipient suited to administration to a patient. In the case of water as the carrier, the water is highly purified and processed to be free of contaminants, e.g., endotoxins.

The deliverable compound may be made in a form suitable for oral, rectal, topical, intravenous injection, intra-articular injection, parenteral administration, intra-nasal, or tracheal administration. Carriers include solids or liquids, and the type of carrier is chosen based on the type of administration being used. Suitable binders, lubricants, disintegrating agents, coloring agents, flavoring agents, flow-inducing agents, and melting agents may be included as carriers, e.g., for pills. For instance, an active component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol and the like. The compounds can be administered orally in solid dosage forms, such as capsules, tablets, and powders, or in liquid dosage forms, such as elixirs, syrups, and suspensions. The active compounds can also be administered parentally, in sterile liquid dosage forms. Buffers for achieving a physiological pH or osmolarity may also be used.

The invention in one aspect relates to the treatment of conditions. The terms “treating” and “treatment” as used herein refer to reduction in severity and/or frequency of symptoms, elimination of symptoms and/or underlying cause, prevention of the occurrence of symptoms (prophylaxis) and/or their underlying cause, and improvement or remediation of damage. Thus, for example, the present method of “treating” a condition encompasses both prevention of the condition in a predisposed individual, treatment of the condition in a clinically symptomatic individual and treatment of a healthy individual for beneficial effect.

“Prophylaxis” or “prophylactic” or “preventative” therapy as used herein includes preventing the condition from occurring or ameliorating the subsequent progression of the condition in a subject that may be predisposed to the condition but has not yet been diagnosed as having it.

As used herein, “condition” refers to any deviation from normal health and includes a disease, disorder, defect or injury, such as injury caused by trauma, and deterioration due to age, inflammatory, infectious or genetic disorder or due to environment.

Conditions in which IL-1Ra administration is beneficial are disclosed in the prior art. Of those, conditions that may be treated in accordance with the present invention fall generally into the categories of those requiring tissue regeneration, particularly those in which increased chondrocyte, collagen, proteoglycan, cartilage or muscle mass form or function is desirable.

Chondrocytes are the only cells found in cartilage. They produce and maintain the cartilaginous matrix, which consists mainly of Type II collagen, proteoglycans and elastin.

Cartilage is a flexible connective tissue found in many areas in the bodies of humans and animals, including joints between bones, rib cage, ear, nose, elbow, knee, ankle, bronchial tubes and intervertebral discs. Unlike other connective tissues, cartilage does not contain blood vessels and thus has limited repair capabilities. Because chondrocytes are bound in lacunae, they cannot migrate to damaged areas. Therefore, if cartilage is damaged, it is difficult and slow to heal.

For the purpose of the present disclosure, conditions that can be treated include Chondrocyte-Related Conditions that will benefit from repair or new growth of cartilage tissue or chondrocytes. This is not exclusive however and is used descriptively to emphasise the benefit of the presently disclosed methods.

Chondrocyte-Related Conditions include joint disorders involving cartilage damage and include cartilage damage caused by tibial plateau decompression.

The cause of osteoarthritis is multifactorial and includes body habitus, genetics and hormonal status.

In osteoarthritis, the cartilage covering bones (articular cartilage—a subset of hyaline cartilage) is thinned, eventually completely wearing out, resulting in a “bone against bone” joint, reduced motion and pain. Current therapeutic modalities are aimed at reducing pain and increasing joint function. Non-invasive interventions such as exercise and weight loss are the first lines of treatment, followed by anti-inflammatory medications. These latter treatments alleviate the symptoms but do not inhibit the processes that result in the changes characteristic of this disease and may actually accelerate joint destruction. Failure of these treatments usually culminates in surgical intervention (arthroplasty). Joint replacement is extremely successful with respect to restoring patient mobility and decreasing pain. However, failure as a result of osteolysis and aseptic loosening due to effects of wear debris or biomechanically-related bone loss limit the lifetime of these implants necessitating higher-risk revision surgery at the expense of increased patient morbidity and failure rate. The present invention provides a treatment for osteoarthritis.

In traumatic rupture or detachment, the cartilage in the knee is frequently damaged, and can be partially repaired through knee cartilage replacement therapy.

In achondroplasia, reduced proliferation of chondrocytes in the epiphyseal plate of long bones during infancy and childhood results in dwarfism.

Costochondritis is an inflammation of cartilage in the ribs, causing chest pain.

In spinal disc herniation, an asymmetrical compression of an intervertebral disc ruptures the sac-like disc, causing a herniation of its soft content. The hernia often compresses the adjacent nerves and causes back pain.

In relapsing polychondritis, a destruction, probably autoimmune, of cartilage, especially of the nose and ears, causes disfiguration. Death occurs by suffocation as the larynx loses its rigidity and collapses.

Tumours made up of cartilage tissue, either benign or malignant, can occur.

The present invention provides a treatment for each of the conditions above. Any of these conditions can be treated by repairing or growing new cartilage or chondrocytes according to the methods disclosed herein utilising a fusion protein according to the present invention.

Other conditions that may be treated in accordance with the invention include: chondromalacia patella; chondromalacia; chondrosarcoma—head and neck; chondrosarcoma; costochondritis; enchondroma; hallux rigidus; hip labral tear; osteochondritis dissecans (OCD); osteochondrodysplasias; perichondritis; polychondritis; or torn meniscus.

The invention provides means to improve the function of existing chondrocytes and cartilage in maintaining a cartilaginous matrix. It also provides means to promote growth of chondrocytes and cartilage and provide a cartilaginous matrix, with or without an implant or prosthesis. In one embodiment the invention provides means to promote cartilage formation or repair in a cellular scaffold or in tissue engineering techniques, for example for cartilage generation or repair to grow new cartilage tissue in tissues including the nose, septum, ear, elbow, knee, ankle and invertebrate discs.

In one aspect the fusion protein is administered with an implant or the like to produce or repair chondrocytes or cartilage tissue that may interact with the implant to treat a condition as disclosed herein. As used herein, “interact” refers to the effect in conjunction of components to achieve a desired biological outcome.

While not wishing to be bound by theory, when an implant “interacts” with chondrocytes, the effect of the implant in treating the condition is greater than the effect of the implant alone and may be synergistic.

In one aspect the fusion protein is administered in combination with growth factors to enhance the regenerative activity of growth factors or reduce desensitisation of cells to growth factor stimulation. The effect of treatment with the fusion protein and growth factors may be more than the additive effect of the separate treatments and may be synergistic.

As used herein, “growth factors” are proteins that regulate many aspects of cellular function, including survival, proliferation, migration and differentiation. Growth factors determine the fate of cells as they differentiate from being progenitors along either neuronal or glial lineages. In addition, during embryonic development, growth factors are crucial for regulating neuronal survival, determining cell fate and establishing proper connectivity. Growth factors typically act as signalling molecules between cells. They often promote cell differentiation and maturation, which varies between growth factors. For example, platelet-derived growth factor BB (PDGF BB) enhances osteogenic differentiation, while fibroblast growth factors and vascular endothelial growth factors stimulate blood vessel differentiation (angiogenesis).

Growth factors that may be administered in combination with the fusion protein of the invention to enhance the regenerative activity of the growth factors or reduce desensitisation of cells to growth factor stimulation include platelet derived growth factor (PDGF), FGF, VEGF and bone morphogenic protein (BMP), particularly PDGF-BB, VEGF-A and BMP-2.

In this embodiment, the “desired biological outcome” provided by the invention is preferably wound healing or bone or cartilage repair and bone or cartilage growth, more preferably removal of the symptoms of osteoarthritis and most preferably treatment and prevention of osteoarthritis.

It is also contemplated that fusion proteins of the invention can be used to promote muscle growth, to improve recovery of muscle from injury, trauma or use, to improve muscle strength, to improve exercise tolerance, to increase the proportion of muscle, to increase muscle mass, decrease muscle wasting, improve muscle repair, or may be useful to treat disorders of muscle including wasting disorders, such as cachexia, and hormonal deficiency, anorexia, AIDS wasting syndrome, sarcopenia, muscular dystrophies, neuromuscular diseases, motor neuron diseases, diseases of the neuromuscular junction, and inflammatory myopathies in a subject in need thereof.

The invention extends to treatment of disorders of muscle and of diseases associated with muscular degeneration characteristics. Non-limiting examples of such disorders are various neuromuscular diseases, cardiac insufficiency, weakness of single muscles such as e.g. the constrictor or bladder muscle, hypo- or hypertension caused by problems with the constrictor function of vascular smooth muscle cells, impotence/erectile dysfunction, incontinence, AIDS-related muscular weakness, and general and age-related amyotrophia.

Disorders of muscle as referred to herein particularly include muscle wasting conditions or disorders in which muscle wasting is one of the primary symptoms.

“Muscle wasting” refers to the progressive loss of muscle mass and/or to the progressive weakening and degeneration of muscles, including the skeletal or voluntary muscles which control movement, cardiac muscles which control the heart, and smooth muscles. In one embodiment, the muscle wasting condition or disorder is a chronic muscle wasting condition or disorder. “Chronic muscle wasting” is defined herein as the chronic (i.e. persisting over a long period of time) progressive loss of muscle mass and/or to the chronic progressive weakening and degeneration of muscle. Chronic muscle wasting may occur as part of the aging process.

The loss of muscle mass that occurs during muscle wasting can be characterized by a muscle protein breakdown or degradation, by muscle protein catabolism. Protein catabolism occurs because of an unusually high rate of protein degradation, an unusually low rate of protein synthesis, or a combination of both. Protein catabolism or depletion, whether caused by a high degree of protein degradation or a low degree of protein synthesis, leads to a decrease in muscle mass and to muscle wasting. The term “catabolism” has its commonly known meaning in the art, specifically an energy burning form of metabolism.

Muscle wasting can occur as a result of age, a pathology, disease, condition or disorder. In one embodiment, the pathology, illness, disease or condition is chronic. In another embodiment, the pathology, illness, disease or condition is genetic. In another embodiment, the pathology, illness, disease or condition is neurological. In another embodiment, the pathology, illness, disease or condition is infectious. As described herein, the pathologies, diseases, conditions or disorders directly or indirectly produce a wasting (i.e. loss) of muscle mass, that is a muscle wasting disorder.

Also contemplated is the treatment of neuromuscular diseases which are aligned with joint or skeletal deformities. In one embodiment, muscle wasting in a subject is a result of the subject having a muscular dystrophy; muscle atrophy; or X-linked spinal-bulbar muscular atrophy (SBMA).

The muscular dystrophies are genetic diseases characterized by progressive weakness and degeneration of the skeletal or voluntary muscles that control movement. The muscles of the heart and some other involuntary muscles are also affected in some forms of muscular dystrophy. The major forms of muscular dystrophy (MD) are: Duchenne muscular dystrophy, myotonic dystrophy, Becker muscular dystrophy, limb-girdle muscular dystrophy, facioscapulohumeral muscular dystrophy, congenital muscular dystrophy, oculopharyngeal muscular dystrophy, distal muscular dystrophy and Emery-Dreifuss muscular dystrophy.

Muscular dystrophy can affect people of all ages. Although some forms first become apparent in infancy or childhood, others may not appear until middle age or later. Duchenne MD is the most common form, typically affecting children. Myotonic dystrophy is the most common of these diseases in adults.

Muscle atrophy (MA) is characterized by wasting away or diminution of muscle and a decrease in muscle mass. For example, Post-Polio MA is a muscle wasting that occurs as part of the post-polio syndrome (PPS). The atrophy includes weakness, muscle fatigue, and pain. Another type of MA is X-linked spinal-bulbar muscular atrophy (SBMA—also known as Kennedy's Disease). This disease arises from a defect in the androgen receptor gene on the X chromosome, affects only males, and its onset is in adulthood.

Sarcopenia is a debilitating disease that afflicts the elderly and chronically ill patients and is characterized by loss of muscle mass and function. Further, increased lean body mass is associated with decreased morbidity and mortality for certain muscle-wasting disorders. In addition, other circumstances and conditions are linked to, and can cause muscle wasting disorders. For example, studies have shown that in severe cases of chronic lower back pain, there is paraspinal muscle wasting.

Muscle wasting and other tissue wasting is also associated with advanced age. It is believed that general weakness in old age is due to muscle wasting. As the body ages, an increasing proportion of skeletal muscle is replaced by fibrous tissue. The result is a significant reduction in muscle power, performance and endurance.

Long term hospitalization due to illness or injury, or disuse deconditioning that occurs, for example, when a limb is immobilized, can also lead to muscle wasting, or wasting of other tissue. Studies have shown that in patients suffering injuries, chronic illnesses, burns, trauma or cancer, who are hospitalized for long periods of time, there is a long-lasting unilateral muscle wasting, and a decrease in body mass.

Injuries or damage to the central nervous system (CNS) are also associated with muscle wasting and other wasting disorders. Injuries or damage to the CNS can be, for example, caused by diseases, trauma or chemicals. Examples are central nerve injury or damage, peripheral nerve injury or damage and spinal cord injury or damage. In one embodiment CNS damage or injury comprise Alzheimer's diseases (AD); stroke, anger (mood); anorexia, anorexia nervosa, anorexia associated with aging and/or assertiveness (mood).

In another embodiment, muscle wasting or other tissue wasting (e.g. tendons or ligaments) may be a result of alcoholism.

In one embodiment, the wasting disease, disorder or condition being treated is associated with chronic illness

This embodiment is directed to treating, in some embodiments, any wasting disorder, which may be reflected in muscle wasting, weight loss, malnutrition, starvation, or any wasting or loss of functioning due to a loss of tissue mass.

In some embodiments, wasting diseases or disorders, such as cachexia, including cachexia caused by malnutrition, tuberculosis, leprosy, diabetes, renal disease, chronic obstructive pulmonary disease (COPD), cancer, end stage renal failure, emphysema, osteomalacia, or cardiomyopathy, may be treated by the methods of this invention

In some embodiments, wasting is due to infection with enterovirus, Epstein-Barr virus, Herpes zoster, HIV, trypanosomes, influenza, coxsackie, rickettsia, trichinella, schistosoma or mycobacteria.

Cachexia is weakness and a loss of weight caused by a disease or as a side effect of illness. Cardiac cachexia, i.e. a muscle protein wasting of both the cardiac and skeletal muscle, is a characteristic of congestive heart failure. Cancer cachexia is a syndrome that occurs in patients with solid tumours and haematological malignancies and is manifested by weight loss with massive depletion of both adipose tissue and lean muscle mass.

Cachexia is also seen in COPD, acquired immunodeficiency syndrome (AIDS), human immunodeficiency virus (HIV)-associated myopathy and/or muscle weakness/wasting is a relatively common clinical manifestation of AIDS. Individuals with HIV-associated myopathy or muscle weakness or wasting typically experience significant weight loss, generalized or proximal muscle weakness, tenderness, and muscle atrophy.

Untreated muscle wasting disorders can have serious health consequences. The changes that occur during muscle wasting can lead to a weakened physical state resulting in poor performance of the body and detrimental health effects.

Thus, muscle atrophy can seriously limit the rehabilitation of patients after immobilizations. Muscle wasting due to chronic diseases can lead to premature loss of mobility and increase the risk of disease-related morbidity. Muscle wasting due to disuse is an especially serious problem in elderly, who may already suffer from age-related deficits in muscle function and mass, leading to permanent disability and premature death as well as increased bone fracture rate. Despite the clinical importance of the condition few treatments exist to prevent or reverse the condition. The inventors propose that the fusion proteins of the invention can be used to prevent, repair and treat muscle wasting or atrophy associated with any of the conditions recited above.

In a preferred embodiment the fusion protein is used to treat burns and sepsis.

The invention in other aspects also contemplates treating healthy individuals to cause an increase in muscle mass, strength, function or overall physique.

The term “increase in muscle mass” refers to the presence of a greater amount of muscle after treatment relative to the amount of muscle mass present before the treatment.

The term “increase in muscle strength” refers to the presence of a muscle with greater force generating capacity after treatment relative to that present before the treatment.

The term “increase in muscle function” refers to the presence of muscle with greater variety of function after treatment relative to that present before the treatment.

The term “increase in exercise tolerance” refers to the ability to exercise with less rest between exercise after treatment relative to that needed before the treatment.

A muscle is a tissue of the body that primarily functions as a source of power. There are three types of muscles in the body: a) skeletal muscle—striated muscle responsible for generating force that is transferred to the skeleton to enable movement, maintenance of posture and breathing; b) cardiac muscle—the heart muscle; and c) smooth muscle—the muscle that is in the walls of arteries and bowel. The methods of the invention are particularly applicable to skeletal muscle but may have some effect on cardiac and or smooth muscle. Reference to skeletal muscle as used herein also includes interactions between bone, muscle and tendons and includes muscle fibres and joints.

In a preferred embodiment the fusion proteins of the invention are used to treat conditions such as skin wound healing, (including diabetic wounds and ulcers), skin burns, bone defects and fractures, osteoporosis, osteoarthritis, spinal fusion, ankle fusion, muscle and tendon defects, cartilage defects and degeneration, ischemic tissues (including ischemic limb, ischemic cardiac tissue, and ischemic brain after a stroke).

Fusion proteins according to the invention may be administered by any suitable route, and the person skilled in the art will readily be able to determine the most suitable route and dose for the condition to be treated and the subject. Fusion proteins may be administered orally, sublingually, buccally, intranasally, by inhalation, transdermally, topically, intra-articularly or parenterally in dosage unit formulations containing conventional non-toxic pharmaceutically acceptable carriers, adjuvants, and vehicles. The term parenteral as used herein includes subcutaneous, intravenous, intramuscular, intrathecal, intracranial, injection or infusion techniques.

In one embodiment the fusion protein may be administered with or in an implant, medical device or prosthesis. The implant may be a biodegradable implant or slow release depot or other implant as known to persons skilled in the art. Such embodiment is particularly appropriate for improving muscle growth and strength after muscle trauma or damage. In one embodiment the fusion protein is capable of delivering IL-1Ra to its intended site of action, e.g. a wound and providing a sustained release of IL-1Ra without a carrier or delivery vehicle.

When used to treat burns the fusion protein may be administered orally, topically or parenterally.

Compositions comprising the fusion protein are to be administered in a therapeutically effective amount. As used herein, an “effective amount” is a dosage which is sufficient to reduce to achieve a desired biological outcome. The desired biological outcome may be any therapeutic benefit including an increase in muscle mass, an increase in muscle strength, muscle growth, or treatment of burns or wounds. Such improvements may be measured by a variety of methods including those that measure lean and fat body mass (such as duel ray scanning analysis), muscle strength, or the formation of muscle cells.

A typical daily dosage might range from about 1 μg/kg to up to 100 mg/kg or more, depending on the mode of delivery.

Dosage levels of the fusion protein could be of the order of about 0.1 mg per day to about 50 mg per day or will usually be between about 0.25 mg to about 1 mg per day. The amount of fusion protein which may be combined with the carrier materials to produce a single dosage will vary, depending upon the subject to be treated and the particular mode of administration. For example, a formulation intended for administration to humans may contain about 1 mg to 1 g of the fusion protein with an appropriate and convenient amount of carrier material, which may vary from about 5 to 95 percent of the total composition. Dosage unit forms will generally contain between from about 0.1 mg to 50 mg of active ingredient.

It will be understood, however, that the specific dose level for any particular subject will depend upon a variety of factors including the activity of the specific compound employed, the age, body weight, general health, sex, diet, time of administration, route of administration, rate of excretion, drug combination and the severity of the particular disease undergoing therapy.

Dosage schedules can be adjusted depending on the half-life of the fusion protein, or the severity of the subject's condition.

Generally, the compositions are administered as a bolus dose, to maximize the circulating levels of peptide for the greatest length of time after the dose. Continuous infusion may also be used after the bolus dose.

In one embodiment a single dose of the fusion protein is delivered locally, optionally in combination with a biomaterial. If required a further dose of fusion protein may be delivered after a period of time selected from one week, two weeks, three weeks, four weeks, one month, two months, three months, 6 months or a year or more.

The treatments of the present invention are suitable for subjects in need thereof. “Subject,” as used herein, refers to human and non-human animals.

The term “non-human animals” includes all vertebrates, e.g., mammals, such as non human primates (particularly higher primates), sheep, horse, dog, rodent (e.g., mouse or rat), guinea pig, goat, pig, cat, rabbits, cow, and non-mammals, such as chickens, amphibians, reptiles, etc. In one embodiment, the subject is an experimental animal, an animal suitable as a disease model, or in animal husbandry (animals as food source), where methods to increase lean muscle mass will greatly benefit the industry. Additionally, the method is particularly important in race horses.

In one embodiment the treatment is for humans, particularly adult humans, children aged 11 to 16 years old, aged 4 to 10 years old, infants of 18 months up to 4 years old, babies up to 18 months old. The treatment may also be used for elderly or infirm humans.

In one embodiment the treatments of the present invention are used to supplement alternative treatments for the same condition. For example, the fusion proteins can be used to supplement stem cell therapies for joint and muscle repair.

### EXAMPLES

The invention described herein will be more readily understood by reference to the following examples which are included merely for purposes of illustration of certain aspects and embodiments of the present disclosure and are not intended to limit the invention in any way.

### Example 1

### IL-1Ra Fused to PIGF₁₂₃₋₁₄₁ Displays Supper-Affinity to ECM Components

Because diabetic mice deficient for IL-1R1 close wound much faster, the inventors proposed delivering IL-1Ra directly in the wounds to promote wound closure. As a positive control, they used PDGF-BB which is a clinically relevant growth factor that is well-known to promote healing of chronic wounds. To optimize the delivery of IL-1Ra and PDGF-BB, the inventors chose to enhance the affinity of the recombinant proteins for endogenous extracellular matrix (ECM) components by fusing them to an ECM-binding sequence derived from the heparin-binding domain of placenta growth factor (PIGF₁₂₃₋₁₅₂).

Firstly, the inventors determined the smallest ECM-binding sequence from PIGF₁₂₃₋₁₅₂, by producing seven truncated version of PIGF₁₂₃₋₁₅₂ and testing their binding to common ECM proteins (fibronectin, vitronectin, tenascin C, and fibrinogen) and heparan sulfate.

### Recombinant PIGF Fragment Production and Purification

PIGF fragment sequences were cloned into the expression vector pGEX6P-1 (GE Healthcare). To purify the proteins, a histidine tag (6×His) was added at the C-terminus of fragments. The fragments were expressed in E. coli BL21 (DE3). Bacteria were cultured overnight in 10 ml lysogeny broth (LB) medium with 100 μg/ml of ampicillin overnight. Then the culture was diluted 1:100 in 250 ml of LB medium with 100 μg/ml of ampicillin and cultured at 37° C. for 3 h. Protein production was induced with 1 mM of isopropyl β-D-1-thiogalactopyranoside overnight at 25° C. Then, the culture was centrifuged at 4,000 g for 10 min. The pellets were resuspended in cold PBS with 1 tablet of protease inhibitor cocktail (Roche), 50 mg of lysozyme (Roche). The solution was sonicated for 20 s with maximum amplitude for 3-4 cycles. Benzonase (500 U, Millipore), 1 mM MgCl2, and 1% Triton X-100 was added and the solution was incubated on a rotor for 30 min at 4° C. Lysate was centrifuged at 12,000 g for 10 min and the supernatant filtered through a 0.22 μm filter. Proteins were first purified using a GSTrap HP 5 ml and secondly with a HisTrap HP 5 ml (GE Healthcare) affinity columns. Chaperone proteins were removed by using an ATP buffer (50 mM Tris-HCl, 150 mM NaCl, 10 mM MgSO₄, 2 mM ATP, pH 7.4). A Triton X-114 buffer (PBS with 0.1% Triton X-114) was used to remove lipopolysaccharides. The final protein solution was dialyzed against PBS and filtered through a 0.22 μm filter. The fragments were verified as >99% pure by SDS-PAGE and stored at −80° C.

### Binding of PIGF Fragments to ECM Proteins

ELISA plates (96-Well Medium Binding, Greiner bio-one) were coated with solutions of 100 nM of human plasma fibronectin (Sigma), human vitronectin (Peprotech), human tenascin C (R&D Systems), or human fibrinogen (Enzyme Research Laboratories) in PBS for 1 h at 37° C. Wells were washed with washing buffer (PBS-T, PBS with 0.05% Tween-20) and blocked with 1% BSA in PBS-T for 1 h at room temperature. Then, wells were incubated with 100 nM of GST-fused PIGF fragments (in PBS-T with 0.1% BSA) for 1 h at room temperature. GST (100 nM) was used as negative control. Wells were washed 3 times with washing buffer and incubated with 0.1 μg/ml of HRP-conjugated antibody against GST (GE Healthcare, RPN1236V) in PBS-T with 0.1% BSA for 45 min at room temperature. Wells were washed 3 times with PBS-T and detection was done with tetramethylbenzidine substrate and measurement of the absorbance at 450 nm.

### Binding of PIGF Fragments to Heparan Sulfate

Heparin-binding plates (Corning, #354676) were coated with 25 fag/ml of heparan sulfate (Sigma-Aldrich, H7640) overnight at room temperature and blocked with a PBS solution containing 0.2% gelatin and 0.5% BSA for 1 h at room temperature. Then, plates were washed 3 times with a washing buffer (100 mM NaCl, 50 mM NaAc, 0.2% Tween-20, pH 7.2). GST-fused PIGF fragments (100 nM in PBS with 0.5% BSA) were added and incubated for 1 h at room temperature. Plates were washed 3 times with the washing buffer and bound GST fragments were detected with HRP-conjugated antibody against GST (0.1 μg/ml in PBS-T with 0.1% BSA; GE Healthcare, RPN1236V). Wells were washed 3 times with PBS-T and detection was done with tetramethylbenzidine substrate and measurement of the absorbance at 450 nm.

The inventors found that a shorter version of the ECM-binding sequence, PIGF₁₂₃₋₁₄₁, strongly binds all ECM protein tested as well as heparan sulfate (FIG. 1 A, B). Then, we engineered IL-1Ra and PDGF-BB with PIGF₁₂₃₋₁₄₁ at their C-terminus to generate IL-1Ra/PIGF₁₂₃₋₁₄₁ and PDGF-BB/PIGF₁₂₃₋₁₄₁ (FIG. 1C).

### Recombinant Protein Production and Purification

IL-1Ra/PIGF₁₂₃₋₁₄₁ and PDGF-BB/PIGF₁₂₃₋₁₄₁ were designed to have a 6× histidine tag at their N-terminus and the PIGF₁₂₃₋₁₄₁ sequence at the C-terminus. Recombinant proteins were produced in E. coli BL21 (DE3) via pET-22b (Novagen). Bacteria were cultured overnight in 10 ml LB medium with 100 μg/ml of ampicillin overnight. Then the culture was diluted 1:100 in 1 l of LB medium with 100 μg/ml of ampicillin and incubated at 37° C. for 3 h. Protein production was induced with 1 mM of isopropyl β-D-1-thiogalactopyranoside overnight at 25° C. Then, the culture was centrifuged at 4,000 g for 10 min. Following protein production and bacterial lysis, the soluble fraction of IL-1Ra/PIGF₁₂₃₋₁₄₁ was purified by affinity chromatography using a chelating SFF(Ni) column with an extensive Triton X-114 wash (0.1% v/v) to remove endotoxins. PDGF-BB/PIGF₁₂₃₋₁₄₁ was extracted from inclusion bodies using a solubilization buffer (50 mM Tris, 6 M GuHCl, 10 mM DTT, pH 8.5). The extracted proteins were then added drop by drop in a refolding buffer (50 mM Tris, 1 mM GSH, 0.1 mM GSSG, pH 8.2) at 4° C., over 4 days, for a final protein solution to buffer ratio of 1:100. The refolded proteins were then purified by affinity chromatography using a chelating SFF(Ni) column with an extensive Triton X-114 wash (0.1% v/v) to remove endotoxins. The fraction containing dimers were pulled together. IL-1Ra/PIGF₁₂₃₋₁₄₁ was stored in PBS with 5 mM EDTA while PDGF-BB/PIGF-2₁₂₃₋₁₄₁ was stored in 4 mM HCl. Murine wild-type IL-1Ra and PDGF-BB were purchased from Peprotech.

### Binding-Affinity of PIGF₁₂₃₋₁₄₁-Fused IL-1Ra and PDGF-BB for ECM Proteins

ELISA plates (Medium binding, Greiner bio-one) were coated with 100 nM of ECM proteins in 50 μl of PBS for 1 h at 37° C.; human plasma fibronectin (Sigma), human vitronectin (Peprotech), human tenascin C (R&D Systems), human fibrinogen (Enzyme Research Laboratories). Then, wells were washed with PBS-T (PBS with 0.05% Tween-20) and blocked with 300 l PBS-T containing 1% BSA for 1 h at room temperature. ECM and control wells (no ECM and blocked with BSA) were further incubated 1 h at room temperature with solutions of murine PDGF-BB (Peprotech), IL-1Ra (R&D Systems), or PIGF₁₂₃₋₁₄₁-fused proteins at concentrations ranging from 0 to 100 nM (50 μl in PBS-T containing 0.1% BSA; PROKEEP tubes, Watson bio lab). Then, wells were washed 3 times with PBS-T and bound PDGF-BB and IL-1Ra variants were detected using biotinylated antibodies in PBS-T containing 0.1% BSA. Antibodies used were from PDGF-BB DuoSet ELISA (R&D Systems, DY8464) for PDGF-BB and IL-1ra/IL-1F3 DuoSet ELISA (R&D Systems, DY480) for IL-1Ra. To calculate the dissociation constants (K_{D}) specific-binding values were fitted with a one site specific binding model using A₄₅₀ nm=Bmax*[growth factors or IL-1Ra variants]/(K_{D}+[growth factors or IL-1Ra variants]).

Remarkably, the engineered proteins displayed 4 to 100-fold increase in affinity for the ECM proteins (Table 1, FIG. 2). Moreover, fusing PIGF₁₂₃₋₁₄₁ to IL-1Ra and PDGF-BB did not alter their activity. Dermal fibroblast proliferation in response to wild-type PDGF-BB and PDGF-BB/PIGF₁₂₃₋₁₄₁ was similar (FIG. 3). Likewise, the ability of IL-1Ra and IL-1Ra/PIGF₁₂₃₋₁₄₁ to inhibit the macrophage response to IL-1β was comparable (FIG. 3).

TABLE 1
IL-1Ra and PDGF-BB engineered with PIGF₁₂₃₋₁₄₁
display super affinity for ECM proteins.

| | Fibronectin K_{d} | Vitronectin K_{d} | Tenascin C K_{d} | Fibrinogen K_{d} |
|---|---|---|---|---|
| Recombinant proteins | (nM) | (nM) | (nM) | (nM) |
| IL-1Ra | 129.0 ± 7.1 | 73.2 ± 23.8 | 112.8 ± 13.0 | 662.7 ± 214.1 |
| IL-1Ra/PIGF₁₂₃₋₁₄₁ | 21.9 ± 13.1* | 0.7 ± 0.2* | 20.7 ± 7.5* | 43.5 ± 12.9* |
| PDGF-BB | 67.5 ± 10.3 | 32.1 ± 3.6 | 94.3 ± 20.6 | 206.2 ± 85.7 |
| PDGF-BB/PIGF₁₂₃₋₁₄₁ | 17.7 ± 2.0** | 1.4 ± 1.0*** | 25.0 ± 2.9** | 16.7 ± 2.0* |

Dissociation constants (K_{d}) in nM are shown. K_{d} values of PIGF₁₂₃₋₁₄₁-fused proteins are all significantly lower compared to wild-type proteins.
Data are means ± SEM. n = 3 independent experiments.
Two-tailed Student's t-test.
*P ≤ 0.05, **P ≤ 0.01, ***P ≤ 0.001.

Next, we assessed the ability of IL-1Ra and PDGF-BB to bind the ECM using an ECM-mimetic hydrogel composed of fibrinogen, fibronectin, vitronectin, tenascin C, and heparan sulfate (FIG. 1D).

Release Assay from ECM-Mimetic Hydrogel

ECM-mimetic hydrogels (50 μl) were generated from a HEPES solution (20 mM, 150 nM NaCl, pH 7.4) containing 8 mg/ml human fibrinogen (Enzyme Research Laboratories), 1 mg/ml human plasma fibronectin (Sigma), 500 μg/ml human vitronectin (Peprotech), 50 ug/ml human tenascin C (R&D Systems), 50 ug/ml heparan sulfate (Sigma) and 500 ng/ml of PDGF-BB or IL-1Ra variants. Matrices were polymerized in Ultra Low Cluster 96-well plate (Corning) at 37° C. for 2 h with 10 U/ml bovine thrombin (Sigma) and 5 mM CaCl₂). Then, matrices were transferred to Ultra Low Cluster 24-well plate (Corning) containing 500 μl of buffer (20 mM Tris-HCl, 150 mM NaCl, 0.1% BSA, pH 7.4). Control wells that served as 100% released control contained only PDGF-BB and IL-1Ra variants in 500 μl of buffer. Every 24 h, buffers were removed, kept at −20° C. and replaced with fresh buffer. For the 100% release control well, 20 μl of buffer was taken out every d and stored at −20° C. After 7 d, the cumulative release of PDGF-BB and IL-1Ra variants was quantified by ELISA using the 100% released control as reference (PDGF-BB DuoSet, IL-1Ra/IL-1F3 DuoSet; R&D Systems). For release assays with plasmin, the same method was used except that the release buffer contained 100 μU/ml of plasmin (Roche).

Both IL-1Ra/PIGF₁₂₃₋₁₄₁ and PDGF-BB/PIGF₁₂₃₋₁₄₁ were retained in the hydrogel while the wild-type forms were quickly released. Moreover, IL-1Ra/PIGF₁₂₃₋₁₄₁ and PDGF-BB/PIGF₁₂₃₋₁₄₁ were gradually released in the presence of the protease plasmin which cleaves the ECM proteins that form the hydrogel as well as PIGF₁₂₃₋₁₄₁ (FIG. 1E).

Lastly, we tested the retention of the engineered proteins after intradermal administration, to conform binding to ECM in vivo.

### Mice

Wild-type C57BL/6 mice were obtained from the Monash Animal Research Platform. BKS.Cg-Dock7^{m}+/+Lepr^{db}/J (Lepr^{db/db)} mice were obtained from the Jackson Laboratory. Because Lepr^{db/db} mice are sterile, Lepr^{db/+} mice were crossed to Il1r1^{-/-} mice³⁸ to obtain fertile Lepr^{db/+}-Il1r1^{-/+} mice. Then, Lepr^{db/+}-Il1r1^{-/+} were crossed together to obtain Lepr^{db/db}-Il1r1^{-/-} mice. Animals were kept under specific pathogen-free conditions. All animal experiments were conducted in accordance with Monash University guidelines and approved by the local ethics committee.

### Intradermal Retention Assay

Back of C56BL/6 mice (8-week-old) were shaved and 10 μl of 6× histidine-tagged wild-type (IL-1Ra-His, Sapphire Biosciences) or IL-1Ra/PIGF₁₂₃₋₁₄₁ in PBS were injected intra-dermally. Injection sites were marked with and mice were euthanized directly after (100% control) or after 1, 3, 5, and 8 d. Full-thickness skin tissue was harvested and the area of the injection site was collected with a 6 mm biopsy punch. Tissue was transferred into 500 μl of T-PER Tissue Protein Extraction Reagent (Thermo Fisher Scientific) containing a protease inhibitor cocktail (1 tablet for 50 ml, Roche) and minced. Samples were incubated for 1 h at room temperature under agitation and centrifugated at 5,000 g for 5 min and supernatants were stored at −80° C. Concentration of IL-1Ra-His or IL-1Ra/PIGF₁₂₃₋₁₄₁ was determined by ELISA utilizing an anti-histidine tag capture antibody (Abcam, ab18184) and a detection antibody form IL-1Ra/IL-1F3 DuoSet DuoSet ELISA kit (R&D Systems). The percent retention was calculated using the d 0 concentrations as the 100%.

Remarkably, compared to the wild-type forms, IL-1Ra and PDGF-BB fused to PIGF₁₂₃₋₁₄₁ showed much longer retention in tissue with about 50% retained after five days (FIG. 1F).

### Discussion:

Efficiently delivering biologics such as growth factors and cytokines in chronic wounds and more generally for regenerative medicine applications has demonstrated to be very challenging, due to their low stability and a short window of activity following the delivery. These issues can be solved by delivering much higher doses, but high doses of growth factors and cytokines may trigger serious adverse effects. In addition, the use of high doses of therapeutics makes therapy likely less cost-effective and therefore less scalable. For example, recombinant IL-1Ra (anakinra, Kineret) is approved for the treatment of rheumatoid arthritis and neonatal-onset multisystem inflammatory disease. Yet, IL-1Ra needs to be used at very high doses (more than 100 mg per injection) with multiple administrations and its usage can lead to side effects such as immunogenicity. The use of high doses of recombinant PDGF-BB (becaplermin, Regranex) has been approved to heal chronic wounds, but the product raised major concerns regarding safety and cost-effectiveness. Therefore, better delivery systems need to be developed to ensure precise localization and retention of low doses of these drugs at the delivery sites. One of the strategies is to engineer the recombinant proteins to strongly bind a biomaterial carrier or the endogenous ECM of the tissue where they are delivered. We have previously shown that engineering growth factors to bear the ECM-binding sequence of PIGF confers super-affinity to ECM components. Here, we thought of designing super-affinity IL-1Ra and PDGF-BB in a similar fashion, to allow retention in the wound and gradual release via proteases that cleave the ECM-binding sequence¹². While we have previously reported PIGF₁₂₃₋₁₄₄ as the ECM-binding sequence, we found here that the shorter sequence, PIGF₁₂₃₋₁₄₁, binds ECM components more strongly. Therefore, we used PIGF₁₂₃₋₁₄₁ to design super-affinity IL-1Ra and PDGF-BB and demonstrated that both engineered factors can be retained into an ECM-mimetic hydrogel and into skin tissue for an extended time.

To evaluate the capacity of super-affinity IL-1Ra to promote wound healing, we compared the engineered antagonist to super-affinity PDGF-BB, since the use of PDGF-BB has been shown to be one of the most promising strategies to promote healing of chronic wounds. A previous study reported that very high doses of wild-type IL-1Ra (750 μg of anakinra) delivered with a gelatin-transglutaminase gel accelerates wound closure in diabetic mice. Here, we found that wound treatment with low dose of wild-type IL-1Ra (0.5 μg) delivered once without a biomaterial carrier has only a marginal effect on diabetic mouse wound closure. However, we demonstrated the same dose of super-affinity IL-1Ra was able to significantly accelerate healing with wound nearly closed one-week post-treatment. Interestingly, treatment with super-affinity IL-1Ra was even faster at promoting wound closure compared to the same dose of super-affinity PDGF-BB (0.5 μg), indicating that delivering immunomodulatory signals may be more important than delivering morphogens. One of the problems that could arise when delivering IL-1Ra is the risk of dampening the immune response in case of bacterial infection. Nevertheless, the use of antiseptics and systemic antibiotics are common in chronic wounds management and low doses of super-affinity IL-1Ra would not have a systemic effect, because of its ability to localize only at the delivery site.

### Example 2: IL-1Ra/PIGF₁₂₃₋₁₄₁ Fusion Protein for Treating Chronic Diabetic Wounds

Chronic wounds have become a major challenge to healthcare systems worldwide potentially affecting a number of at-risk populations including diabetic patients, elderly patients, and those that remain bedridden. While the causes leading to impaired wound healing are relatively diverse, chronic wounds have features in common such as excessive levels of pro-inflammatory immune cells and cytokines, high concentration of proteases, low levels of growth factors, and higher number of senescent cells. Because of these cellular and molecular characteristics, chronic wounds are usually trapped in the inflammation phase of the healing process and fail to progress through the normal phases of healing in an orderly and timely manner. The persistent inflammation preventing the progression of chronic wounds to an anti-inflammatory and repair state is likely due to dysregulation of immune signalling. Indeed, inadequate immune responses and imbalance in immune signalling are very common in diabetic and elderly patients, where high levels of the pro-inflammatory cytokine interleukin-1 (IL-1) plays a central role. For example, obesity and hyperglycaemia are known to induce expression IL-1β in a number of different cell types, including immune cells such as macrophages.

The impact of IL-1β in chronic wounds is still elusive, but IL-1β is known to act as an upstream signal for sustaining inflammasome activity in wound macrophages, in addition to induce inflammatory signals in other cell types. Since activation of the inflammasome promotes the release of IL-1β, the pro-inflammatory cytokine could be part of an inflammatory positive-feedback loop that prevents polarization of macrophages towards an anti-inflammatory phenotype. More generally, an excess of IL-1β-driven inflammatory signals in wounds may trigger a cascade of events that prevent wound closure. For instance, these events could include slow clearance of inflammatory cells, senescence of fibroblasts, and degradation of pro-repair growth factors and extracellular matrix proteins due to high levels of matrix metalloproteinases (MMPs). Therefore, blocking IL-1β or IL-1 receptor (IL-1R1) signalling may be an interesting option to reduce the persistent inflamed condition in chronic wounds and promote healing.

In this study, we first used a knockout mouse model to evaluate the actual impact of IL-1R1 signalling in diabatic would healing. We demonstrate that IL-1R1 prevents wound closure in diabetic conditions. Then, we optimized a protein engineering system to deliver low doses of recombinant IL-1 receptor antagonist (IL-1Ra) in a localized and sustained manner using an IL-1Ra/PIGF fusion protein that binds to the extracellular matrix, removing the need for using a biomaterial carrier. We show that wound treatment with the IL-1Ra fusion protein promotes wound healing by re-establishing a healing microenvironment in diabetic mice, characterized by lower levels of pro-inflammatory cells, cytokines and senescent fibroblasts, and higher levels of anti-inflammatory cytokines and growth factors. In addition, the engineered IL-1Ra fusion protein was also surprisingly superior to a growth factor-based treatment with engineered platelet-derived growth factor-BB (PDGF-BB). Engineered IL-1Ra has translational potential for chronic wounds and other inflammatory conditions where IL-1R1 signalling needs to be dampened.

### Methods:

### Macrophage Isolation and Stimulation

Bone marrow from femora and tibiae of C57BL/6 mice (8-week-old) was flushed out with Dulbecco's Modified Eagle Medium/Nutrient Mixture (DMEM/F12 medium, Gibco) using a 27-gauge needle and a syringe. Cells were filtered through a 70 μm nylon strainer, centrifuged at 500 g for 10 min at 4° C., and resuspended in DMEM/F12 medium containing 10% heat-inactivated FBS, 100 mg/ml penicillin/streptomycin, and 20 ng/ml murine M-CSF (R&D Systems). Cells were plated in 150 mm diameter petri dishes at a density of 5×10⁷ and cultured for 7 d at 37° C. and 5% CO₂. Medium was replaced every 3 d. After 7 d, macrophages were detached using TrypLE (Gibco) containing 3 mM EDTA and seeded in 12-well plates at a density of 2×10⁵ cells per well in DMEM/F12 with 10% heat-inactivated FBS and 100 mg/ml penicillin/streptomycin. For measurement of secreted IL-1β, macrophages were unstimulated or stimulated with 100 ng/ml LPS (InvivoGene) for 3 h followed by 5 mM ATP (InvivoGene) for 21 h. The concentration of IL-1 released in the media was measure by ELISA (IL-1 DuoSet ELISA kit, R&D Systems). For macrophage stimulation with IL-1β, cells were co-stimulated with IL-1β (1 ng/ml) and IL-1Ra variants at increasing concentrations (0 to 1 μg/ml of IL-1Ra or equimolar concertation of IL-1Ra/PIGF₁₂₃₋₁₄₁). After 24 h, the concentration of IL-6 released in the media was measure by ELISA (IL-6 DuoSet ELISA kit, R&D Systems).

### Blood Glucose Measurements

Non-fasted blood glucose levels were measured in 10-week-old Lepr^{db/db}, Lepr^{db/+}-Il1r1^{-/+}, and C57BL/6 mic. Briefly, tail veins were pricked and small blood samples (2-5 μl) were collected followed by measurement of blood glucose concentration using a FreeStyle Optium Neo meter (Abbott). Any values exceeding the maximum readout of the glucometer was recorded at 500 mg/dl.

### Skin Wound Healing Model

Male mice (12 to 14-week-old) backs were shaved and four full-thickness punch-biopsy wounds (5 mm in diameter) were created as described in Mochizuki, M et al., (2019) Nat Biomed Eng. After 10 minutes, the wounds were treated with 10 μl saline (PBS) or protein solution in PBS (0.5 μg IL-1Ra, 0.61 μg IL-1Ra/PIGF₁₂₃₋₁₄₁, 0.5 μg PDGF-BB, 0.65 μg PDGF-BB/PIGF₁₂₃₋₁₄₁). For non-diabetic mice (Lepr^{db/+}) only two wounds per mice were created, due to their smaller size compared diabetic mice. The wounds were covered with non-adhering dressing (Adaptic, Johnson & Johnson) and adhesive film dressing (Hydrofilm, Hartmann). At specific time points post-wounding, animals were euthanized and the wounds were harvested for biochemical or histological analysis.

### Histological Analysis

Wounds were harvested with an 8 mm tissue biopsy punch and fixed in 10% neutral buffered formalin for 24 h at room temperature. The samples were trimmed until the edge of the wound, embedded in paraffin, and serially sectioned onto 4 μm slides until the centre of the wound was passed. The extent of re-epithelialization was measured by histomorphometric analysis. Slides were stained with hematoxylin and eosin and wound centres were determined by measuring the distance between the panniculus carnosus muscle gap using Aperio ImageScope Viewer (Germany). The extent of wound closure was calculated as the ratio between the epidermis closure over the length of the panniculus carnosus gap.

### Biochemical Analysis of the Wounds

Area of 8 mm in diameter were excised from the centre of the wounds, minced with scissors, and incubated for 1 h at room temperature in T-PER Tissue Protein Extraction Reagent (500 μl per wound, Thermo Fisher Scientific) containing 1 tablet of protease inhibitor for 10 ml (Roche). Samples were then centrifuged at 5,000 g for 5 min and supernatants were stored at −80° C. Total protein concentration was measured with a Bradford assay (Millipore). Cytokines, growth factors and MMPs and TIMP-1 were detected by ELISA from R&D Systems; Mouse IL-1ra/IL-1F3 DuoSet ELISA ELISA; Mouse IL-1 beta/IL-1F2 DuoSet ELISA; Mouse IL-6 DuoSet ELISA; Mouse CXCL1/KC DuoSet ELISA; Mouse FGF basic/FGF2/bFGF DuoSet ELISA; Mouse/Rat PDGF-BB DuoSet ELISA; Mouse VEGF DuoSet ELISA; Mouse TGF-beta 1 DuoSet ELISA; Mouse IL-10 Quantikine ELISA Kit; Mouse IL-4 DuoSet ELISA; Total MMP-2 Quantikine ELISA Kit; Mouse Total MMP-9 DuoSet ELISA; Mouse TIMP-1 DuoSet ELISA.

### Dermal Fibroblast Isolation

C57BL/6J mice tails were cut off completely from the base followed by a brief sterilization procedure by submerging tails in 70% ethanol for 2 min. An incision was made along the midline, throughout the length of the tail from the base to the tail tip, and the skin was peeled off from the bone. Tails were rinsed in PBS, cut into 1-2 cm² pieces and incubated with 2 mg/ml ice-cold dispase II (Sigma) at 4° C. for 10-12 h. Then, skin pieces were washed, and the epidermis along with hair follicles was peeled off. The dermal pieces were minced digested in with 300 U/ml collagenase II (Sigma) for 30 min at 37° C. The supernatant was collected and filtered with a 100 μm filter and inactivated with EDTA (5 mM). Cells were centrifuged at 500 g for 10 min and plated on T75 flasks (1 flask per tail) in fibroblast media (DMEM with 2 mM GlutaMAX, 1 mM sodium pyruvate, 10% heat-inactivated FBS, 100 units/ml penicillin and 100 mg/ml streptomycin). All cells were used within the first 3 passages for experiments.

### Proliferation Assay

Dermal fibroblasts (passage 4) were starved for 24 h in low-serum α-MEM (100 mg/ml penicillin/streptomycin, 2 mM glutamax, 2% FBS). Then, cells were seeded in a 96-well plate (2,000 cells/well) with low-serum α-MEM containing PDGF-BB variants. Percentage of new cells was calculated over basal proliferation (low-serum α-MEM only) using Cyquant (Thermo Fisher Scientific) and the equation ((cell number in basal proliferation group/cell number in stimulation group)−1)×100.

### In Vivo Phenotyping of Neutrophils and Macrophages

Mice (12-week-old) were wounded as described above and euthanized after 6, 9, and 14 d. The back skin was excised and wounds were harvested with an 8 mm biopsy punch and placed into RPMI media 1040 (Gibco) and 2% BSA. Wounds were minced with scissors and digested with collagenase XI for 30 min at 37° C. Enzymatic digestion was neutralized with 5 mM EDTA and the mixture was passed through a 70 μm cell strainer. Single cell suspensions were stained for 20 min on ice using with antibodies from Biolegends (2 μg/ml): BV711 anti-F4/80 (clone BM8), biotin anti-CD11b (clone M1/70), PE-Cy7 anti-CD206 (clone C0862C), BV421 anti-Ly6G (clone 1A8). UV379-streptavidin was from BD Biosciences. Fixable live/dead viability staining was done using Zombie Aqua Fixable Viability Kit (Biolegend). All flow cytometry was performed on an BD LSR Fortessa X20 flow cytometer and analysed using FlowJo (Tree Star).

### Senescence-Associated β-Galactosidase Activity

Mice (12-week-old) were wounded as described above and euthanized after 9 d. Injured skin was harvested using an 8 mm biopsy and healthy skin tissue was collected from an uninjured. Samples were minced and digested with 1.5 mg/ml Collagenase XI (Sigma), at 37° C. for 45 min twice. Collagenase was inactivated with 5 mM EDTA and the supernatant was passed through a 70 μm filter. Cells were first stained with LIVE/DEAD Fixable Aqua Dead Cell Stain Kit (1:500 diluted in PBS, Invitrogen) for 30 min at 4° C., followed by incubation with 10 μg/ml TruStain FcX anti-CD16/32 (clone 93, BioLegend) antibodies diluted in flow cytometry buffer (PBS with 5% FBS and 5 mM EDTA). Then, cells were stained with BV711 conjugated Biolegend rat anti-mouse antibodies against CD11b (2 ug/ml, clone M1/70), F4/80 (5 ug/ml, clone BM8) and CD45 (1 ug/ml, clone 30-F11) diluted in flow cytometry buffer, for 30 min at 4° C. SA-3-gal staining was performed using CellEvent Senescence Green Flow Cytometry Assay Kit (Invitrogen, #C10840), according to the manufacturer's instructions. Then, cells were washed with PBS containing 1% BSA and permeabilised with 0.25% Triton X-100 in 1% BSA in PBS for 15 min at room temperature. Cells were washed with flow cytometry buffer containing 0.25% saponin, and further stained with Abcam rabbit monoclonal antibodies against Vimentin (0.2 μg/ml, clone EPR3776), Hsp47 (25 μg/ml, clone EPR4217) and S100A4 (7.5 μg/ml, clone EPR14639(2)) for 45 min at 4° C., followed by staining with secondary antibody Alexa Fluor® 647-conjugated goat anti-rabbit IgG (Abcam, #ab150083) for 45 min at 4° C. Finally, cells were washed and resuspended in flow cytometry buffer for analysis on BD LSRFortessa X-20. The post-acquisition analysis was done using FlowJo software (Tree Star Inc.). For the in vitro experiment, fibroblasts (passage 2) were cultured in 10% FBS with IL-1β (1 ng/ml) or PBS control. Media was changed twice at d 3 and 6. At d 9, cells were stained with LIVE/DEAD Fixable Aqua Dead Cell Stain Kit and CellEvent Senescence Green Flow Cytometry Assay Kit.

### Statistical Analysis

All data are presented as means±SEM. Statistical analyses were performed using GraphPad Prism 8 statistical software (GraphPad). Significant differences were calculated with Student's t-test or by analysis of variance (ANOVA), followed by the Bonferroni post hoc test when performing multiple comparisons between groups. P<0.05 was considered as a statistically significant difference. The symbols *, **, and *** indicate P values less than 0.05, 0.01, and 0.001 respectively; n.s., not significant.

### Results:

### IL-1R1 Signalling Impairs Wound Healing in Diabetic Mice

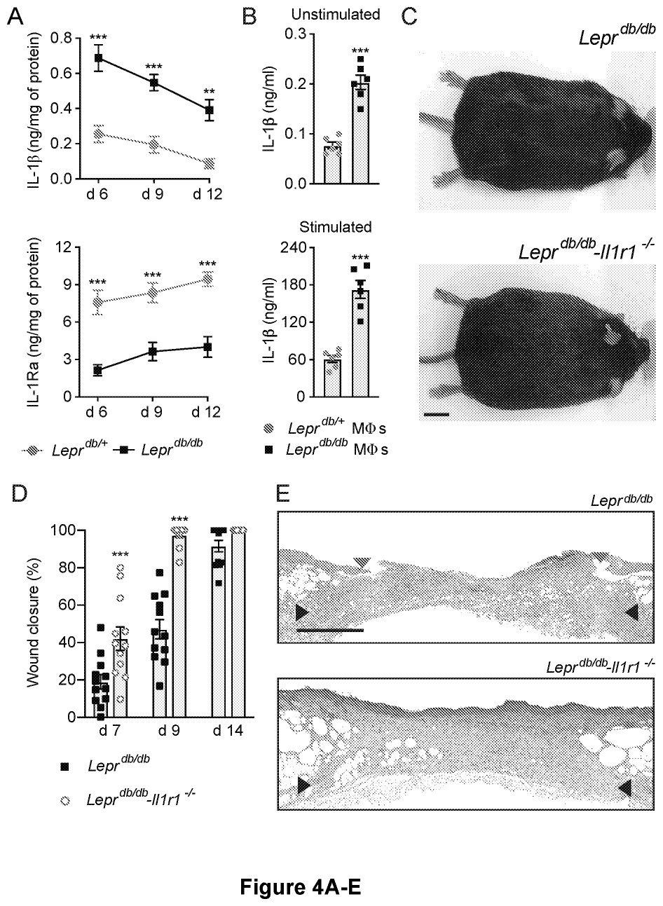

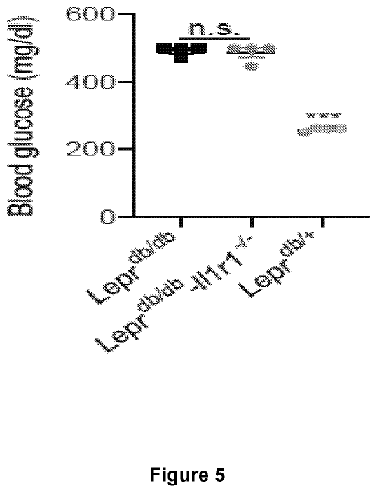

IL-1R1 signalling is tightly controlled by IL-1 receptor antagonist (IL-1Ra) and a high ratio between IL-1β and IL-1Ra in tissues leads to robust IL-1R1 signalling. Thus, we first tested if the ratio between IL-1β and IL-1Ra differs between skin wounds of non-diabetic and diabetic mice. We measured the concentrations of IL-1β and IL-1Ra in wounds of Lepr^{db/db} (diabetic) and of Lepr^{+/db} (non-diabetic littermates) mice at various time points post-injury. At every time point, we found significantly elevated levels of IL-1β in diabetic mice compared to non-diabetic mice, as well as significantly lower levels of IL-1Ra (FIG. 4A). Macrophages derived from circulating monocytes accumulating in injured tissues are known to be a significant source of IL-1β. Therefore, we compared IL-1β production by Lepr^{db/db} and Lepr^{+/db} monocyte-derived macrophages. Lepr^{db/db} macrophages significantly secreted more IL-1β in steady-state and following stimulation with lipopolysaccharides (LPS) and adenosine triphosphate (ATP) (FIG. 4B). Then, to evaluate the actual impact of IL-1R1 signalling during wound healing in diabetic mice, we crossed Lepr^{db/db} mice with Il1r1^{-/-} to obtain diabetic mice deficient for IL-1R1 signalling (Lepr^{db/db}-Il1r1^{-/-}) (FIG. 4C; FIG. 5). Interestingly, diabetic mice deficient for IL-1R1 signalling healed wounds much faster with near-complete re-epithelization after 9 d, while diabetic wounds were approximately 50% open (FIG. 4 D, E).

### Super-Affinity IL-1Ra Promotes Wound Healing in Diabetic Mice

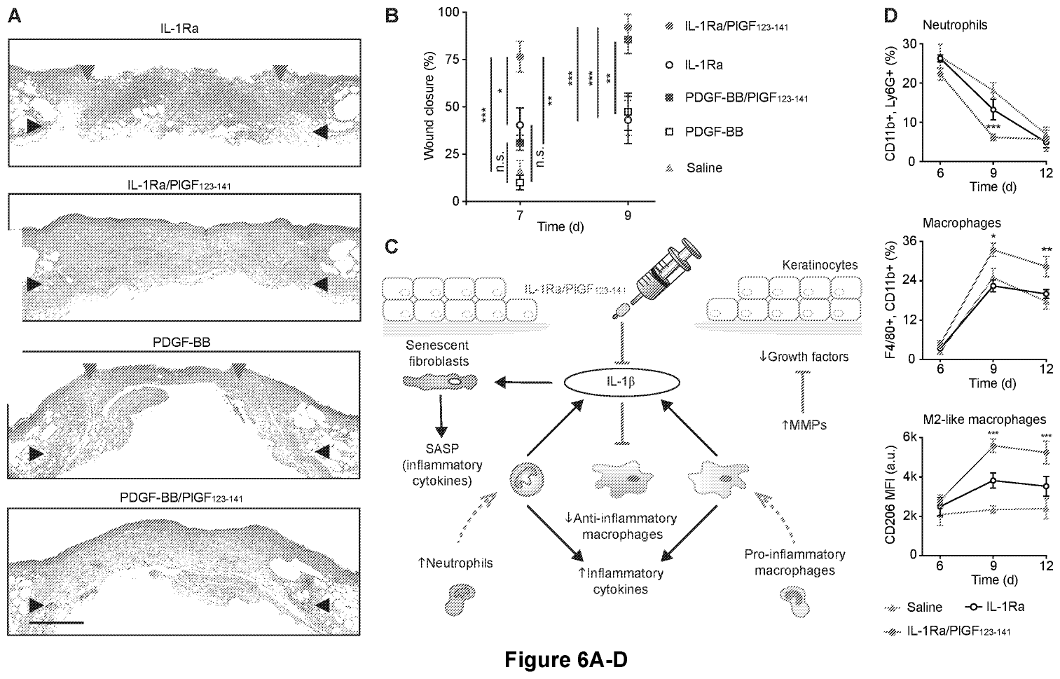

To assess whether localized delivery of IL-1Ra promotes healing of diabetic wounds, we chose the Lepr^{db/db} mouse model again. Low doses of IL-1Ra variants and PDGF-BB variants were delivered topically in saline only once following full-thickness wounding (0.5 μg of IL-1Ra and PDGF-BB, and equimolar doses of the engineered forms). One week after treatment, wounds that received IL-1Ra/PIGF₁₂₃₋₁₄₁, showed significantly more closure—characterized by the extent of re-epithelization—compared to saline control, IL-1Ra, and both forms of PDGF-BB (FIG. 6A,6B). Near 100% closure was observed 9 d after treatment, in wounds that received IL-1Ra/PIGF₁₂₃₋₁₄₁ and PDGF-BB/PIGF₁₂₃₋₁₄₁, while wounds treated with saline, IL-1Ra and PDGF-BB were still largely open (FIG. 6A,6B).

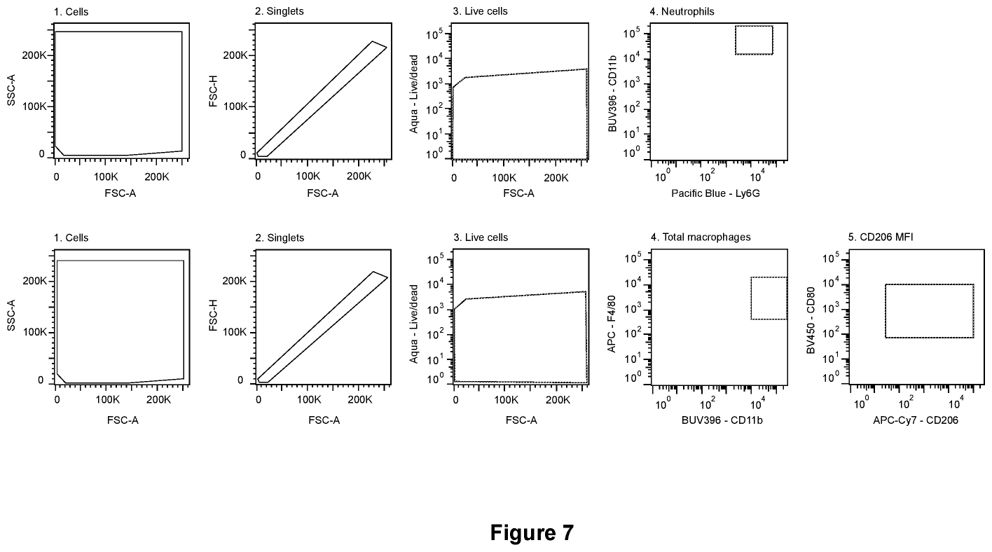

Based on these results, we hypothesized that treatment with IL-1Ra would lead to a series of molecular events resulting from the inhibition of IL-1β (FIG. 6C). IL-1β triggers a strong inflammatory response that induces the mobilization of inflammatory neutrophils and macrophages. Therefore, we tested to which extent IL-1Ra/PIGF₁₂₃₋₁₄₁ modulates neutrophil and macrophage accumulation in wounds during the healing process, using flow cytometry (FIG. 7). Interestingly, treatment of wounds with IL-1Ra/PIGF₁₂₃₋₁₄₁, promoted faster clearance of neutrophils (CD11 b⁺, Ly6G⁺ cells) and accumulation of more macrophages (F4/80⁺, CD11 b⁺ cells), compared to treatment with wild-type IL-1Ra or saline (FIG. 6D). Moreover, the expression levels of CD206—a marker for M2-like macrophages—were significantly higher in the IL-1Ra/PIGF₁₂₃₋₁₄₁ treated group, indicating that wound macrophages were likely more anti-inflammatory (FIG. 6D).

### Super-Affinity IL-1Ra Leads to a Pro-Healing Microenvironment

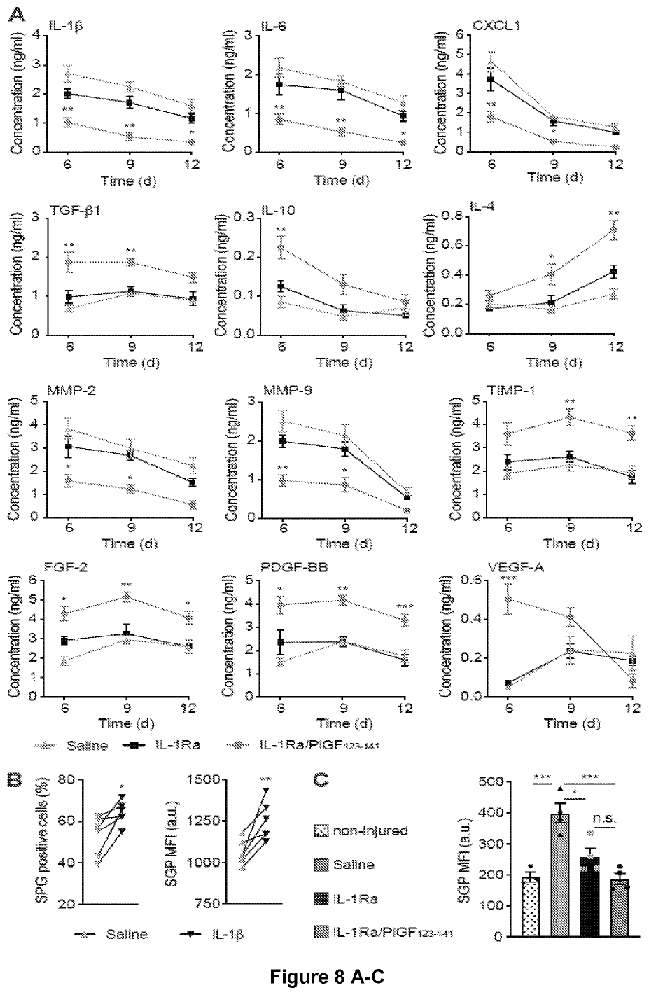

Levels of cytokines, matrix-metalloproteinases (MMPs), and growth factors in the wound microenvironment are critical for the progression of the healing process. Thus, we investigated to which extent treatment with super-affinity IL-1Ra influences the concentration of these factors during wound healing. We first measured wound concentrations of the pro-inflammatory cytokines IL-1β, IL-6, and CXC chemokine ligand (CXCL) 1 (a neutrophil chemoattractant), as well as the anti-inflammatory cytokines transforming growth factor-β1 (TGF-β1), IL-10, and IL-4. Remarkably, compared to treatment with saline and IL-1Ra, IL-1 Ra/PIGF₁₂₃₋₁₄₁ significantly reduced pro-inflammatory factor concentrations in the wounds while increasing the concentrations of the ant-inflammatory factors (FIG. 8A). Next, we measured the levels of MMP-2 and MMP-9 which are usually at excessive concertation in diabetic wounds and are known to degrade ECM components and growth factors. Delivering IL-1Ra/PIGF₁₂₃₋₁₄₁ significantly decreased the levels of MMP-2 and 9 but increased the levels of the MMP inhibitor metallopeptidase inhibitor TIMP-1. Lastly, we focused on fibroblast growth factor-2 (FGF-2), PDGF-BB, and vascular endothelial growth factor-A (VEGF-A) which are key wound healing growth factors secreted by macrophages and other cells. Delivering IL-1Ra/PIGF₁₂₃₋₁₄₁ significantly enhanced the concentration of these pro-healing factors compared to saline and IL-1Ra (FIG. 8A).

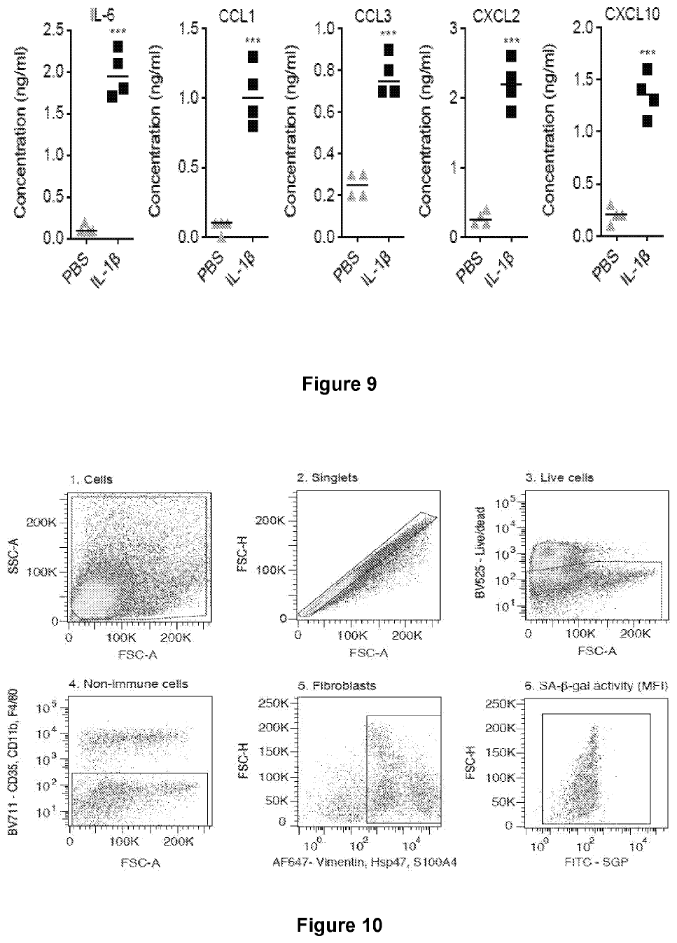

Since fibroblast senescence is a major hurdle in diabetic wounds healing, we explored the effect of IL-1Ra treatment on wound fibroblast senescence. Firstly, we tested the effect of IL-1β on dermal fibroblast in vitro. We decided to measure senescence-associated β-galactosidase (SA-β-gal), as an increase in its activity is considered a hallmark for senescence. Interestingly, we found that treatment of primary dermal fibroblasts with IL-113 increases SA-β-gal activity, suggesting that the cytokine accelerates cellular senescence (FIG. 8B). Confirming that exposure to IL-1β drives senescence, stimulation of fibroblasts with IL-1β induced the secretion of MMP-2 and typical senescence-associated cytokines including IL-6, CC chemokine ligands 1 (CCL1) and 3 (CCL3), as well as CXCL2, and 10 (FIG. 9). Lastly, we tested whether treatment of diabetic wounds with IL-1Ra reduced wound fibroblast senescence. We found that both IL-1Ra variants, but IL-1Ra/PIGF₁₂₃₋₁₄₁, to more extent, decrease SA-β-gal activity in wound fibroblasts. Interestingly, 9 d post-treatment with IL-1Ra/PIGF₁₂₃₋₁₄₁ wound fibroblast displayed a level of SA-β-gal activity similar to dermal fibroblasts found in uninjured skin (FIG. 8C, FIG. 10).

### Discussion

There has been a recent growing interest in developing therapies based on immuno-modulation for chronic wounds and more generally for tissue repair. For instance, a number of strategies have explored the use of biomaterials, cytokines, microRNA, and extracellular vesicles. However, translating these approaches into the clinic may be challenging, due to issues related to safety, scalability, and cost-effectiveness. In addition, a better understanding of the most important molecular pathways through which the immune system prevents or promotes the wound healing process is essential to design novel and effective therapies. Here, we assumed that IL-1R1 signalling via IL-1β is one of the most important pathways preventing the maturation of a pro-repair microenvironment in chronic wounds. As a model, we used full-thickness wound healing in diabetic mice (Lepr^{db/db}), since it is a well-established and widely accepted clinically relevant experimental model of impaired wound healing.

IL-1R1 signalling in tissues is tightly regulated by the ratio between IL-1 and IL-1Ra. Therefore, our first experiment was to measure the IL-18 and IL-1Ra concentrations in wounds of non-diabetic and diabetic mice, to determine if the intensity and duration of IL-1R1 signalling are stronger in the diabetic model. We found that the IL-18:1L-1Ra ratio is continuously higher in Lepr^{db/db} wounds, which therefore indicate that IL-1R1 signalling is probably much more pronounced in the diabetic situation. Interestingly, a similar higher ratio has been reported at the mRNA level in cornea wounds of diabetic mice. Then, we sought to evaluate the real impact of IL-1R1 signalling during wound healing in diabetic mice by generating a diabetic mouse deficient for IL-1R1 (Lepr^{db/db}-IL-1R1^{-/-}). Strikingly, diabetic mice deficient for IL-1R1 healed wounds significantly faster compared to diabetic mice with functional IL-1R1 signalling. Together, these results strongly suggest that IL-1R1 signalling is a key component that prevents wound closure in diabetic mice and it prompted us to explore its inhibition through the delivery of IL-1Ra as a possible treatment to promote wound healing.

Efficiently delivering biologics such as growth factors and cytokines in chronic wounds and more generally for regenerative medicine applications has demonstrated to be very challenging, due to their low stability and a short window of activity following the delivery. These issues can be solved by delivering much higher doses, but high doses of growth factors and cytokines may trigger serious adverse effects. In addition, the use of high doses of therapeutics makes therapy likely less cost-effective and therefore less scalable. For example, recombinant IL-1Ra (anakinra, Kineret) is approved for the treatment of rheumatoid arthritis and neonatal-onset multisystem inflammatory disease. Yet, IL-1Ra needs to be used at very high doses (more than 100 mg per injection) with multiple administrations and its usage can lead to side effects such as immunogenicity. The use of high doses of recombinant PDGF-BB (becaplermin, Regranex) has been approved to heal chronic wounds, but the product raised major concerns regarding safety and cost-effectiveness. Therefore, better delivery systems need to be developed to ensure precise localization and retention of low doses of these drugs at the delivery sites. One of the strategies is to engineer the recombinant proteins to strongly bind a biomaterial carrier or the endogenous ECM of the tissue where they are delivered. We have previously shown that engineering growth factors to bear the ECM-binding sequence of PIGF confers super-affinity to ECM components. Here, we thought of designing super-affinity IL-1Ra and PDGF-BB in a similar fashion, to allow retention in the wound and gradual release via proteases that cleave the ECM-binding sequence. While we have previously reported PIGF₁₂₃₋₁₄₄ as the ECM-binding sequence, we found here that the shorter sequence, PIGF₁₂₃₋₁₄₁, binds ECM components more strongly. Therefore, we used PIGF₁₂₃₋₁₄₁ to design super-affinity IL-1Ra and PDGF-BB and demonstrated that both engineered factors can be retained into an ECM-mimetic hydrogel and into skin tissue for an extended time.

To evaluate the capacity of super-affinity IL-1Ra to promote wound healing, we compared the engineered antagonist to super-affinity PDGF-BB, since the use of PDGF-BB has been shown to be one of the most promising strategies to promote healing of chronic wounds. A previous study reported that very high doses of wild-type IL-1Ra (750 μg of anakinra) delivered with a gelatin-transglutaminase gel accelerates wound closure in diabetic mice. Here, we found that wound treatment with low dose of wild-type IL-1Ra (0.5 μg) delivered once without a biomaterial carrier has only a marginal effect on diabetic mouse wound closure. However, we demonstrated the same dose of super-affinity IL-1Ra is able to significantly accelerate healing with wound nearly closed one-week post-treatment. Interestingly, treatment with super-affinity IL-1Ra was even faster at promoting wound closure compared to the same dose of super-affinity PDGF-BB (0.5 μg), indicating that delivering immunomodulatory signals may be more important than delivering morphogens. One of the problems that could arise when delivering IL-1Ra is the risk of dampening the immune response in case of bacterial infection. Nevertheless, the use of antiseptics and systemic antibiotics are common in chronic wounds management and low doses of super-affinity IL-1Ra would not have a systemic effect, because of its ability to localize only at the delivery site.

To understand the underlying mechanisms by which super-affinity IL-1Ra promotes healing of diabetic wounds, we first focused on wound neutrophils and macrophages, because elevated number of these cells coupled with immune dysregulations contribute to the development of non-healing wounds. For instance, an abnormal high number of neutrophils in chronic wounds leads to an over-production of pro-inflammatory cytokines, reactive oxygen species (ROS), extracellular traps, and proteases (MMPs and serine proteases). Similarly, failure of wound macrophages conversion to an anti-inflammatory phenotype leads to high levels of pro-inflammatory cytokines (e.g. IL-1β and IL-6) and proteases as well as reduction of key growth factors such as VEGF-A, PDGFs and TGF-β1. Altogether, this creates a non-healing microenvironment where wounds cannot progress into the proliferation and resolution phases, due to exacerbation of inflammatory signals combined with ECM degradation and low levels of growth factors. Interestingly, we found that treatment with super-affinity IL-1Ra reduces the neutrophil percentage over total cells in the wounds. Consequently, the percentage of macrophages was up in this group. However, compared to the other groups, the surface expression of CD206 in the macrophages was higher, indicating that polarization towards anti-inflammatory phenotype was more advanced. Further supporting lower levels of inflammatory immune cells following treatment with super-affinity IL-1Ra, the wound concentration of pro-inflammatory cytokines (IL-1β, IL-6, CXCL1) and MMPs (MMP-2, MMP-9) were significantly lower, while anti-inflammatory cytokines (TGF-β1, IL-4, IL-10) and TIMP-1 concentration were higher. The concentrations of wound healing growth factors (FGF-2, PDGF-BB, VEGF-A) were also higher after treatment with super-affinity IL-1Ra. Together, the pro-repair microenvironment induced by super-affinity IL-1Ra treatment can probably be attributed to higher number of growth factor-expressing M2-like macrophages in the wound as well as of lower levels of MMPs which degrades ECM components and growth factors.

Last, we explored the effect of the treatment with super-affinity IL-1Ra on wound fibroblast senescence, because it is a major hurdle in diabetic wound healing. Mechanistically, it has been reported that over-production of ROS by immune cells at the wound site causes ECM and cell membrane damage which result in premature cell senescence. In addition, inflamatory macrophages have been show to activate a senescence program in dermal fibroblast. High levels of senescent dermal fibroblasts further contribute to wound chronicity via having senescence-associated secretome characterized by elevated levels of pro-inflammatory cytokines, chemokines, and proteases. Here, we first showed in vitro that IL-1β increased β-gal activity in dermal fibroblast indicating that the pro-inflammatory cytokine likely accelerates senescence. Further supporting this effect, IL-1β induced the secretion of pro-inflammatory cytokines and chemokines including IL-6, CCL1, CCL3, CXCL2, and CXCL10 which are typically associated with a senescence phenotype. Last, wound treatments with super-affinity IL-1Ra was able to reduce the apparent dermal fibroblast senescence at 9 days post-injury to levels observed in non-inured skin. Together, these results suggest that super-affinity IL-1Ra reduces dermal fibroblast senescence directly by inhibiting IL-1R1 signalling in fibroblasts and indirectly through decreasing neutrophil and inflammatory macrophage levels in the wound.

In conclusion, the inventors demonstrate that IL-1R1 signalling is a major factor that prevents the healing of diabetic wounds. Simple inhibition of IL-1R1 by delivering an engineered matrix-binding form of IL-1Ra that allows precise localization and retention in the wounds without the need of a biomaterial carrier to promote fast wound healing. Wound treatment with engineered IL-1Ra re-establishes a pro-healing microenvironment which is characterized by lower levels of pro-inflammatory cells, cytokines, and senescent fibroblasts and higher levels anti-inflammatory cytokines and growth factors. ECM-binding IL-1Ra holds clinical translational potential for chronic wounds and may also be used in other tissue and inflammatory conditions where IL-1R1 signalling need to be turned down.

### Example 3: Local and Sustained Inhibition of IL-1 Receptor with an Engineered IL-1 Receptor Antagonist Fusion Protein with Binding Affinity to the ECM (IL-1Ra/PIGF₁₂₃₋₁₄₁ Fusion Protein) Augments the Regenerative Effectiveness of Growth Factors in Two Bone Regeneration Models

Growth factors are powerful molecules capable of stimulating a variety of cellular processes including cell proliferation, migration, and differentiation. Therefore, they have raised a lot of hope for regenerative medicine and several growth factor-based products have reached clinical applications. Nevertheless, therapies based on recombinant growth factors are still hindered by limitations that include ineffectiveness at low doses and serious side-effects at high doses. For example, these limitations have led the US Food and Drug Administration (USFDA) to release boxed warning for some growth factors such as bone morphogenetic protein-2 (BMP-2) and platelet-derived growth factor-BB (PDGF-BB). Indeed, the issues that limit growth factors in regenerative medicine are certainly linked to the use of sub-optimal delivery systems. Thus, various strategies have been developed to ensure precise localization of recombinant growth factors in tissues and control their release. However, controlling growth factor localization and release may just be the first step to improve their regenerative potential and safety. Understanding how the signalling of growth factors is affected by the tissue microenvironment once they are released is likely of utmost importance. Importantly, an immune response almost always accompanies the tissue repair regeneration processes but the impact of the immune system and inflammatory signals on growth factor signalling has been overlooked and is poorly understood. In particular, the response of cells to growth factors could change depending on the inflammatory and immune microenvironment at the delivery site. Moreover, although growth factors are delivered with the hope of targeting cells that make new tissues such as tissue-resident stem cells and progenitor cells, they would inevitably also act on immune cells present in damaged tissues, as many of them express growth factor receptors. However, the response of immune cells following a local delivery of recombinant growth factors is still elusive.

The pro-inflammatory cytokine interleukin-1 (IL-1) and its ubiquitously expressed receptor (IL-1R1) are central mediators of innate immunity and inflammation, virtually affecting all cells and organs. Moreover, IL-1R1 has been shown to have a significant impact in the repair and regeneration of various tissues. The inventors investigated the extent to which IL-1R1-mediated pro-inflammatory signalling affects tissue regeneration driven by recombinant growth factors, with the ultimate goal of designing successful regenerative strategies that integrate control of the immune system. As a model system, they used bone regeneration in mice driven by BMP-2 and PDGF-BB, which are two clinically relevant growth factors for regenerative medicine.

### Materials and Methods

### Mice

Wild-type C57BL16 mice were obtained from the Monash Animal Research Platform or Japan SLC. Il1r1^{-/-} mice were backcrossed onto a C57BL/6 background for more than 8 generations. Animals were kept under specific pathogen-free conditions. All animal experiments were conducted in accordance with Monash University guidelines and approved by the local ethics committee or the Animal Research Committee of the Research Institute for Microbial Diseases of Osaka University.

### Calvarial Defect Model

Mice used for surgery were 10-12 weeks old. Mice were anaesthetized with isoflurane and the top of their head was shaved. A longitudinal incision was performed to reveal the skull, and bone tissue was exposed by retracting the soft tissues. Using a drill, two craniotomy defects (4.5 mm diameter) were created in the parietal bones of the skull on each side of the sagittal suture line. The defects were washed with saline and covered with a fibrin matrix polymerized atop the dura (40 μl per defect, 14 mg/ml fibrinogen (Enzyme Research Laboratories), 4 U/ml of bovine thrombin (Sigma), 5 mM CaCl₂), 25 μg/ml aprotinin (Roche)). In selected conditions, 1 μg of growth factor and IL-1Ra variants were added in the matrix. Then, the soft tissue was closed with sutures. As a painkiller, mice received a subcutaneous injection of buprenorphine (0.1 mg/kg), Mice were sacrificed 8 weeks after surgery and the skulls were analyzed by microCT.

### MicroCT

Skulls were scanned with a microCT 40 (Scanco Medical AG) operated at an energy of 55 kVp and intensity of 145 ms for detailed measurements. Scans were reconstructed with a nominal isotropic resolution of 30 μm. After reconstruction, a 3D Gaussian filter (sigma 1.2, support 1) was applied to all images. Bone was segmented from background using a global threshold of 22.4% of maximum grey value. Afterwards, cylindrical masks were placed manually at the defects. Bone coverage and volume within these masks was calculated using the scanner software (IPL, Scanco Medical AG). Coverage was calculated on a dorso-ventral projection of the cylindrical area. Femurs were scanned I ethanol with the same microCT operated at an energy of 55 kVp and intensity of 145 ms and 300 ms integration time for detailed measurements. Scans were performed at high-resolution mode resulting in a nominal isotropic resolution of 15 μm. After reconstruction, a 3D Gaussian filter (sigma 1.2, support 1) was applied to all images. Images were rotated to align the longitudinal axis of the bone to the y-axis of the image. Bone was segmented from background using a global threshold of 31.6% of maximum grey value and a region of interest of 250×320×250 voxels selected centrally at the defect region. Bone volume was calculated using the scanner software (IPL, Scanco Medical AG).

### Compact Bone-Derived MSC Isolation

All muscle and cartilage tissue from long bones (arms and legs) of C57BL/6 mice (6-8 weeks old) were removed and briefly submerged in 70%, before being washed in PBS. The epiphysis of each bone was cut off and bone marrow was flushed using a 27-gauge needle attached to a 10 ml syringe filled with PBS. The cleaned bones were cut into 1-2 mm chips and digested for 1 h at 37° C. in 5 ml α-MEM containing 1 mg/ml collagenase II (Merck). Chips were washed with α-MEM (with 10% FBS), evenly distributed on a cell culture dish (one 10 cm² dish per mouse) and covered with 6 ml culture media (α-MEM, 100 mg/ml penicillin/streptomycin, 2 mM glutamax, 10% FBS). Chips were incubated at 37° C., 5% CO₂ for 10 days allowing cells to migrate out and adhere on the surface. Media was changed at day 5. Cells and chips were washed with PBS, harvested with 0.25% trypsin/EDTA, and transferred to cell culture dishes (two 21 cm² dishes per mouse). Cells were grown to 80% confluence and transferred without the chips in flasks (one 175 cm² flask per mouse). MSCs were expanded until 2 passages with a 1:4 split ratio and stored in liquid nitrogen. MSC phenotype was assessed by staining cells with 1 μg/ml TruStain FcX anti-CD16/32 (clone 93, BioLegend) and LIVE/DEAD Zombie Aqua (1:500 dilution, BioLegend). Then, cells were labelled with the following antibodies from Biolegend; 2 μg/ml anti-CD11b PE (clone M1/70), 5 μg/ml CD45 FITC (clone 30-F11), 1 ug/ml anti-MHCII APC-Cy7 (clone M5/114.15.2), 2 ug/ml anti-CD44 APC-Cy7 (clone 1M7), 2 μg/ml anti-CD29 PE (clone HM61-1), 2 ug/ml anti-CD90.2 APC (clone 30-H12), 2 ug/ml anti-CD140b APC (clone APB5), and 2 ug/ml anti-Sca-1 PE (clone D7). Anti-CD19 APC from BD Pharmigen (clone 1D3) was used at 2 μg/ml. Antibodies were diluted in flow cytometry buffer (PBS, 5 mM EDTA, 1% BSA). Samples were acquired on CyAn ADP (Beckman Coulter) and analyzed with FlowJo software (Tree Star Inc.).

### Osteoblast Isolation

Calvariae of C57BL/6 3 days old mice were digested in α-MEM containing 1 mg/ml collagenase type II (Merck) and 2 mg/ml dispase (Sigma) at 37° C. for 20 min in a shaking water bath to release calvarial cells. The supernatant containing released cells was transferred to a new tube, centrifuged at 300×g, and the pellet was resuspended in α-MEM containing 100 mg/ml penicillin/streptomycin and 10% FBS. The calvariae were digested 3 more times for a total of 4 digestions. Digestions 3 and 4 containing osteoblasts were combined together and transferred in a tissue culture-treated dish at a density of 3×10⁵ cells/ml. Calvarial cells were maintained in culture for 2 weeks in osteogenesis induction medium (α-MEM with 2 mM of L-glutamine, 10% FBS, 100 mg/ml penicillin/streptomycin, 50 mM ascorbate-phosphate, 10 mM β-glycerophosphate, 50 ng/ml of BMP-2) and stored in liquid nitrogen before use.

### Matrix Mineralization

MSCs (passage 3) were seeded in 24-well plates (10,000 cells/well) with 750 μl of osteoblast differentiation medium (α-MEM with 2 mM of L-glutamine, 10% FBS, 100 mg/ml penicillin/streptomycin, 50 mM ascorbate-phosphate, 10 mM β-glycerophosphate) with 50 ng/ml of BMP-2 and/or 1 ng/ml of murine IL-1β (Peprotech). For Smurf2 inhibition experiment, 20 mM heclin (R&D Systems) was added in the medium. After 5 days, osteogenesis inducing medium was replaced without IL-16 or heclin. The medium was changed every 4 days until day 21. To determine the degree of mineralization, medium was removed and the wells washed once with PBS. Cells were fixed with 10% formalin at room temperature for 1 h and wells were washed with water before being stained with 2% alizarin red for 20 min. Alizarin red was aspirated and wells were washed twice with water before being photographed.

### Quantitative PCR for Osteoblastic Differentiation

Cells (MSCs passage 3 or osteoblasts) were seeded in 24-well plates (10,000 cells/well) with 750 μl of osteogenesis inducing medium (α-MEM with 2 mM of L-glutamine, 10% FBS, 100 mg/ml penicillin/streptomycin, 50 mM ascorbate-phosphate, 10 mM β-glycerophosphate) containing BMP-2 (50 ng/ml) with or without murine IL-1β (1 ng/ml). After 5 days, osteogenesis inducing medium was replaced (without IL-18). Media was changed every 4 days. Seven or 14 days following stimulation with IL-18, total RNAs were isolated, using RNeasy Plus Mini Kit (Qiagen) and reverse transcription was performed using ReverTra Ace (Toyobo Co., Ltd.). Quantitative PCR was performed with an ABI PRISM 7500 using TaqMan Assay. The following primers from Applied Biosystems were used: Alpl mouse, Mm00475834_m1; Runx2 mouse, Mm00501580_m1; lbsp mouse, Mm00492555_m1; Eukaryotic 18S rRNA Endogenous Control (VIC/MGB Probe, Primer limited).

### MSC Colony Formation

Fresh MSCs were seeded in 6-well plates (100 cells/well) and grown in 6 ml of α-MEM (containing 100 mg/ml penicillin/streptomycin, 2 mM glutamax and 10% FBS) with or without IL-1β (1 ng/ml) and PDGF-BB (10 ng/ml) for 5 days. Media was changed once and cells were cultured for 5 more days. To assess colony formation, media was aspirated and plates were rinsed with PBS. Then, cells were stained in 3% crystal violet solution in methanol for 10 min. The plates were washed with water until excess crystal violet was removed. The number of colonies (more than 50 cells, determined by light microscopy) was counted and their size was measured using ImageJ software.

### Cell Proliferation Assays

Cells (MSCs passage 3 or osteoblasts) were starved for 24 h in low-serum α-MEM (100 mg/ml penicillin/streptomycin, 2 mM glutamax, 1% FBS). Then, cells were seeded in a 96-well plate (1,500 cells/well) with low-serum α-MEM containing 10 ng/ml PDGF-BB and/or 1 ng/ml of murine IL-10 (Peprotech). For the PHLPP inhibition experiment, medium contained 20 mM of NSC-45586 (Aobious). For the experiment with IL-1Ra variants, 10, 100 or 1,000 ng/ml IL-1Ra (R&D Systems) or IL-1Ra/PIGF₁₂₃₋₁₄₁ was added to the medium. For experiment with PDGF-BB variants, medium contained only 1, 5 or 20 ng/ml of PDGF-BB or PDGF-BB/PIGF₁₂₃₋₁₄₁. Percentage of new cells was calculated over basal proliferation (low-serum α-MEM only) using Cyquant (Thermo Fisher) and the equation ((cell number in basal proliferation group/cell number in stimulation group)−1)×100.

### Migration Assays

Low-serum α-MEM (100 mg/ml penicillin/streptomycin, 2 mM glutamax, 1% FBS) containing 10 ng/ml of murine PDGF-BB with or without 1 ng/ml of murine IL-1β, was added to the bottom side of a collagen type I (C4243, Sigma) coated transwell (8 mm pore size, Millipore). For PHLPP inhibition experiments, medium contained 20 mM of NSC-45586 (Aobious). Directly after, cells (30,000 cells in 200 ml) were added onto the transwell upper chambers. After 6 h, membranes of each insert were removed and mounted on microscopy glass slides using Vectashield mounting medium containing DAPI (Vector Laboratories). The number of cells that migrated to the bottom side of the membrane was counted in 3 random fields using a fluorescent microscope. Fold increase in migration was calculated over basal migration (bottom chamber with medium only).

### Smad Proteins Quantification

Cells (MSCs passage 3 or osteoblasts) were seeded in 6-well plates and cultured until 70% confluency with α-MEM (100 mg/ml penicillin/streptomycin, 2 mM glutamax, 10% FBS). Then, cells were starved for 24 h with low-serum α-MEM before being stimulated with PBS or 1 ng/ml of murine IL-1β (Peprotech). For Smurf2 inhibition experiment, 20 mM heclin (R&D Systems) was added to the medium. After 24 h, Smad1/5/8 relative levels were quantified using a sensitive ELISA kit according to the manufacturer's instructions (Signosis, TE-0015).

### SMURF and PHLPP Quantitative PCRs

Cells (MSCs passage 3 or osteoblasts) were seeded in 6-well plates and cultured until 70% confluency with α-MEM (100 mg/ml penicillin/streptomycin, 2 mM glutamax, 10% FBS). Then, cells were starved for 24 h with low-serum α-MEM before being stimulated with 1 ng/ml of murine IL-1β (Peprotech) for 0 to 72 h (Smurf experiment) or for 0 to 24 h (PHLPP experiment). Total RNA was isolated using RNeasy Plus Mini Kit (Qiagen) and reverse transcription was performed using ReverTra Ace (Toyobo). Quantitative PCR was performed with an ABI PRISM 7500 using TaqMan Assay with the following primers: Smurf1, Mm00547102_m1; Smurf2, Mm03024086_ml; Phlpp1, Mm01295850_m1; Phlpp2 mouse, Mm01244267_m1; Eukaryotic 18S rRNA Endogenous Control (VIC/MGB Probe, primer limited) (Applied Biosystems).

### SMURF2 and PHLPP1 ELISAs

Cells (MSCs passage 3 or osteoblasts) were seeded in 6-well plates and cultured until 70% confluency with α-MEM (100 mg/ml penicillin/streptomycin, 2 mM glutamax, 10% FBS). Then, cells were starved for 24 h with low-serum α-MEM before being stimulated with PBS or 1 ng/ml of murine IL-1β (Peprotech). After 24 h, Smurf2 and PHLPP1 were quantified using ELISA (Mouse E3 Ubiquitin-Protein Ligase SMURF2 and Mouse PH Domain Leucine-Rich Repeat-Containing Protein Phosphatase 1 ELISA Kits, MyBiosource) according to the manufacturer's instructions.

### Akt Phosphorylation Assay

Cells (MSCs passage 3 or osteoblasts) were seeded in 6-well plates and cultured until 70% confluency with α-MEM (100 mg/ml penicillin/streptomycin, 2 mM glutamax, 10% FBS). Cells were starved for 24 h with low-serum α-MEM before being stimulated with PBS or 1 ng/ml murine IL-1β (Peprotech) for 4 h. Then, murine PDGF-BB (10 ng/ml, Peprotech) was added in the medium for 0 to 180 min. For PHLPP inhibition experiments, 20 mM of NSC-45586 (Aobious) was added in the medium. Total Akt and phosphorylated Akt were quantified by ELISA (Phospho-Akt (S473) Pan Specific DuoSet IC, R&D Systems) according to the manufacturer's instructions.

### SA-β-Gal Assay

MSCs (passage 4) were seeded at 3,000 cells/cm² in α-MEM (100 mg/ml penicillin/streptomycin, 2 mM glutamax, 5% FBS) containing IL-1β (1 ng/ml, Peprotech). Media was changed every 3 days and cells were used for senescence analysis on day 5 and day 15. Cells seeded for day 15 were passaged twice during the treatment duration to avoid over-confluency. At the indicated time points, cells were harvested using TrypLE Express (Gibco), and first stained with LIVE/DEAD Fixable Aqua Dead Cell Stain Kit (1:500 diluted in PBS, Invitrogen) for 30 min at 4° C. Then, SA-β-gal activity was measured, using CellEvent Senescence Green Flow Cytometry Assay Kit (Invitrogen, #C10840), according to the manufacturer's instructions. Briefly, cells were fixed with paraformaldehyde (2% in PBS) for 15 min at room temperature. Then, cells were washed with PBS containing 1% BSA, and further stained with the CellEvent Senescence Green Probe (diluted 1:500 in pre-warmed CellEvent Senescence Buffer) for 2 h at 37° C. without CO₂. After incubation, cells were washed with PBS containing 1% BSA and resuspended in flow cytometry buffer (PBS with 1% BSA and 5 mm EDTA) for analysis on BD LSRFortessa X-20. The post-acquisition analysis was done using FlowJo software (Tree Star Inc.).

### Senescence-Associated Cytokine Detection

MSCs (passage 4) were seeded at in 6-well plates at 70% confluency in α-MEM (100 mg/ml penicillin/streptomycin, 2 mM glutamax, 2% FBS) and stimulated with IL-1β (5 ng/ml). After 48 h, cytokines in the media were detected using an antibody array (Mouse Cytokine Array Panel A, R&D Systems) according to the manufacturer's instructions. The assay was done with 400 ml of cell culture supernatant. The chemiluminescent signals were detected using ImageQuant LAS 4000 and quantified with ImageQuant TL software (GE Healthcare Life Sciences).

### Release of IL-1β Following Bone Injury

Calvarial defects (4.5 mm diameter) in C57BL/6 mice were treated with a fibrin matrix as described for the calvarial defect model with 1 mg of murine BMP-2 or PDGF-BB (Peprotech). After 1, 3, 6, 10, and 15 days, the partially remodeled matrix and the bone tissue surrounding the defect (1 mm farther) was collected. As a control a 4.5 mm diameter calvarial bone tissue was collected (day 0). Fibrinous matrices and tissue samples were incubated in 1 ml of tissue protein extraction reagent (T-PER, Thermo Scientific) containing protease inhibitors (1 tablet of protease inhibitor cocktail (Roche) for 10 ml) and homogenized with a tissue homogenizer. Tissue lysates were incubated for 1 h at 4° C. and centrifuged at 5′000×g for 5 min, before being stored at −80° C. Cytokines were detected by ELISA (Mouse IL-1 beta/IL-1F2 DuoSet, R&D Systems) according to the manufacturer's instructions.

### Macrophage Depletion

One day before surgery, 200 μl of 5 mg/ml clodronate liposomes or empty liposomes (Liposoma) was intravenously injected in C57BL6 mice (10-12 weeks old). Additional 200 μl of clodronate liposomes or empty liposomes were intraperitoneally injected every 2 days until day 6. Mouse spleens were harvested, crushed and red blood cells lysed with red blood cell lysis buffer (8.3 g/L ammonium chloride, 10 mM Tris-HCl in distilled water). Macrophage depletion was verified by resuspending splenocytes in 1 μg/ml TruStain FcX anti-CD16/32 (clone 93, BioLegend) antibodies to block non-specific binding and 1:500 dilution of Zombie Aqua (BioLegend) diluted in PBS. Subsequently, splenocytes were labelled with the following antibodies; 1 μg/ml anti-CD11b PE (clone M1/70, BioLegend), 1 μg/ml anti-Ly6G BV421 (clone 1A8, BioLegend) and 3 μg/ml anti-F4/80 Biotin (clone REA126, Miltenyi Biotech) conjugated to 0.4 μg/ml Streptavidin APC/Fire 750 (BioLegend) diluted in flow cytometry buffer (PBS with 1% BSA and 5 mM EDTA). Samples were acquired on a BD FACS Fortessa X20 and analyzed with FlowJo software (Tree Star Inc.).

Macrophage Stimulation with Growth Factors and Detection of Growth Factor Receptors

Bone marrow from femora and tibiae of C57BL/6 mice (8-12 weeks) was flushed out with Dulbecco's Modified Eagle Medium/Nutrient Mixture (DMEM/F12 medium, Gibco) using a 27-gauge needle and a 10 ml syringe. Cells were filtered through a 70 μm nylon strainer, centrifuged at 500×g for 10 min at 4° C., and resuspended in DMEM/F12 medium containing 10% heat-inactivated FBS, 100 mg/ml penicillin/streptomycin, and 20% L929 fibroblasts-conditioned medium. Cells were plated in 150 mm diameter petri dishes at a density of 5×10⁷ and cultured for 7 days at 37° C. and 5% 002. Medium was replaced every 3 days. After 7 days, macrophages were detached using TrypLE (Gibco) containing 3 mM EDTA and seeded in 12-well plates at a density of 2×10⁵ cells per well in DMEM/F12 with 10% heat-inactivated FBS and 100 mg/ml penicillin/streptomycin. After 1 day, macrophages were detached and stained with 1 μg/ml TruStain FcX anti-CD16/32 (clone 93, BioLegend) and LIVE/DEAD Fixable Aqua Dead Cell Stain Kit (1:500 diluted in PBS, Invitrogen) for 30 min at room temperature. Then cells were washed once with PBS and incubated with biotin anti-CD140a (PDGFRα)(10 fag/ml, clone APA5, Biolegend), APC anti-CD140b (PDGFRb)(10 μg/ml, clone APB5, Biolegend), Alexa Fluor 488 anti-BMPR1A (20 μg/ml, C32447, Signalway Antibody), Alexa Fluor 647 anti-BMPR1B (20 μg/ml, C32547, Signalway Antibody), Alexa Fluor 488 anti-BMPR2 (20 μg/ml, C32961, Signalway Antibody), or Alexa Fluor 647 anti-ACVR1 (20 μg/ml, C43627, Signalway Antibody). PE-streptavidin (1 μg/ml, Biolegend) was used with biotin anti-mouse CD140a. Cells were acquired on a LSRFortessa X-20 and analyzed with FlowJo software (Tree Star Inc.).

### Binding of Growth Factors and IL-1β to ECM Proteins

ELISA plates (Greiner bio-one medium binding, Thermo Fisher Scientific) were coated with 100 nM of ECM proteins in 50 ml of PBS for 1 h at 37° C. Then, wells were washed with 400 ml of PBS-T (0.05% Tween-20) and blocked with 300 ml of BSA solution (1% in PBS-T) for 1 h at room temperature. Wells without ECM protein coating, and wells blocked only with the BSA solution were used as controls for non-specific binding. ECM coated wells and control wells were further incubated 1 h at room temperature with solutions of BMP-2 (Peprotech), murine PDGF-BB (Peprotech), IL-1Ra (R&D Systems), or PIGF₁₂₃₋₁₄₁-fused proteins at concentrations ranging from 0 to 100 nM (50 ml in PBS-T containing 0.1% BSA prepared in protein-low bind tubes (PROKEEP, Watson bio lab). Then, wells were washed 3 times with 300 ml of PBS-T and incubated for 1 h with 50 ml of biotinylated polyclonal detection antibodies in PBS-T with 0.1% BSA. Detection antibodies used were from BMP-2 DuoSet ELISA (R&D Systems, DY355) for BMP-2, PDGF-BB DuoSet ELISA (R&D Systems, DY8464) for PDGF-BB and IL-1Ra/IL-1F3

DuoSet ELISA (R&D Systems, DY480) for IL-1Ra. Wells were further washed 3 times with PBS-T and incubated for 20 min with 50 ml of streptavidin-horseradish peroxidase (HRP). After three washes with PBS-T, TMB (ThermoFisher) was added followed by 50 ml of stop solution (0.16M sulfuric acid). Concentrations of detection antibodies and streptavidin-HRP were used according to the manufacturer's instructions. To calculate the dissociation constants (K_{D}) specific-binding values were fitted with a one site specific binding model using A450 nm=Bmax*[growth factors or IL-1Ra variants]/(K_{D}+[growth factors or IL-1Ra variants]).

Release of Growth Factor and IL-1Ra Variants from Fibrin Matrix

Fibrin matrices were generated with 8 mg/ml fibrinogen (Enzyme Research Laboratories), 2 U/ml human thrombin (Sigma), 5 mM CaCl₂, and 500 ng/ml of growth factors or IL-1Ra variants. Fibrin matrices were polymerized at 37° C. for 1 h and transferred to Ultra Low Cluster 24-well plate (Corning) containing 500 ml of buffer (20 mM Tris-HCl, 150 mM NaCl, 0.1% BSA, pH 7.4). Control wells that served as 100% released control contained only the growth factor and IL-1Ra variants in 500 ml of buffer. Every 24 h, buffers were removed, kept at −20° C. and replaced with fresh buffer. For the 100% release control well, 20 ml of buffer was taken out every day and stored at −20° C. After 7 days, the cumulative release of growth factor and IL-1Ra variants was quantified by ELISA using the 100% released control as reference (BMP-2 DuoSet, PDGF-BB DuoSet, IL-1Ra/IL-1F3 DuoSet; R&D Systems). For release assay with MMPs, the same method was used except that the release buffer contained 100 mU/ml of plasmin (Roche).

### Recombinant Growth Factors and IL-1Ra Variants

IL-1Ra/PIGF₁₂₃₋₁₄₁ and PDGF-BB/PIGF₁₂₃₋₁₄₁ were designed to bear a 6×His-tag at their N-terminus and the PIGF₁₂₃₋₁₄₁ sequence at the C-terminus. Recombinant proteins were produced in E. coli via pET-22b (Novagen) and subsequently purified and refolded. Briefly, following protein production and bacterial lysis, the soluble fraction of IL1 Ra; PIGF₁₇₃₋₁₄₁ was purified by affinity chromatography using a chelating SFF(Ni) column with an extensive Triton X-114 wash (0.1% v/v) to remove endotoxins. PDGF-BB/PIGF₁₂₃₋₁₄₁₁ was extracted from inclusion bodies using a solubilization buffer (50 mM Tris, 6M GuHCl, 10 mM DTT, pH 8.5). The extracted proteins were then added drop by drop in a refolding buffer (50 mM Tris, 1 mM GSH, 0.1 mM GSSG, pH 8.2) at 4° C., over 4 days, for a final protein solution to buffer ratio of 1:100. The refolded proteins were then purified by affinity chromatography using a chelating SFF(Ni) column with an extensive Triton X-114 wash (0.1% v/v) to remove endotoxins. The fraction containing dimers were pulled together IL1Ra/PIGF₁₂₃₋₁₄₁ was stored in PBS with 5 mM EDTA while PDGF-BB/PIGF₁₂₃₋₁₄₁ was stored in 4 mM HCl. Murine wild-type IL-1Ra and BMP-2 were purchased from Peprotech and were endotoxin free.

### Macrophage Polarization

Calvarial defects were treated with fibrin matrices as described above. Matrices were functionalized with IL1Ra IL-1Ra/PIGF₁₂₃₋₁₄₁ (250 ng). Mice were sacrificed at days 3, 6, or 9 post-surgery and matrices were harvested along with bone surrounding the defect area (1 mm farther). The harvested material was then mechanically broken down in smaller pieces and digested in collagenase XI (1 mg/ml, Gibco) at 37° C. After 30 min. 500 μl of serum was added and the digested material was passed through a 70 μm cell strainer and centrifuged. Cells were washed in PBS and labeled with LIVE/DEAD Zombie Aqua (1:500 dilution, BioLegend) in PBS. Then, cells were washed once with PBS and surface staining was performed for 15 min in PBS with 2% heat inactivated FBS. Cells were fixed using FoxP3 fixation and permeabilization kit (eBioScience) and incubated with the following antibodies form Biolegends; anti-F4/80 FTC (clone BM8), anti-CD11b PE (clone M1/70). anti-CD80 BV421 (clone 16-10A1), and anti-CD206 PE-Cy7 (clone C068C2). Samples were acquired on CyAn ADP (Beckman Coulter) and data were analyzed with FlowJo software (Tree Star Inc.).

### Long Bone Defect

Mice (C57BL/6) used for surgery were 14 weeks old. Femoral defects and stabilization were made using the MouseFix plate 6-hole system, purchased from RISystem (RIS.401.130). In brief, mice were anaesthetized with isoflurane. Their left hind limb was shaved and scrubbed for aseptic surgery using iodine wipes. The skin and the fascia lata were incised from the hip joint to the knee and the vastus lateralis and biceps femoris were split to expose the full length of the femur. The stabilization plate was fixed by four screws on the femur and a 2 mm osteotomy was performed using a saw guide and wire saw. The defects were filled with a fibrin matrix polymerized between the restricted ends of the femur (40 μl per defect, 14 mg/ml fibrinogen (Enzyme Research Laboratories), 2U/ml of thrombin (Sigma-Aldrich), 5 mM CaCl₂, 25 μg/ml aprotinin (Roche)). The matrices were functionalized with 1 μg BMP-2 and 1 μg PDGF-BB/PIGF₁₂₃₋₁₄₁ with or without 1 μg IL-1Ra/PIGF₁₂₃₋₁₄₁. Mice were sacrificed 12 weeks after surgery and femurs were analyzed by microCT.

### Results

### The Regenerative Effect of BMP-2 and PDGF-BB is Enhanced in Il1r1^{-/-} Mice

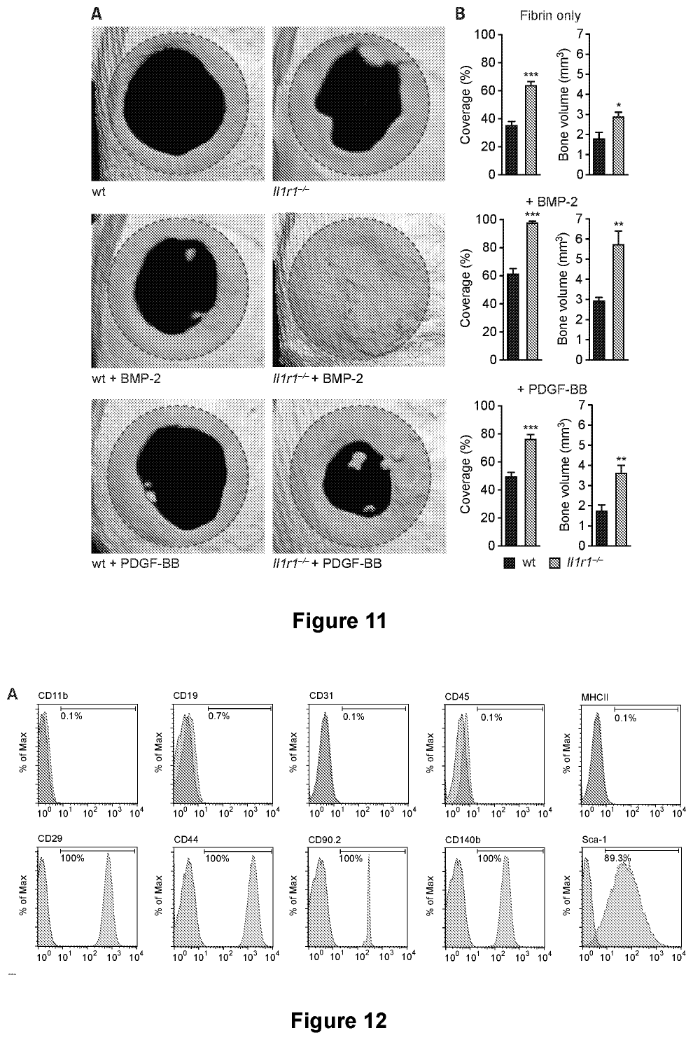

To evaluate the importance of IL-1R1 during bone regeneration driven by local delivery of growth factors, we first analyzed the regenerative capacity of recombinant BMP-2 and PDGF-BB in the IL-1R1 knockout mouse. As a typical bone regeneration model, we used critical-size calvarial defects that we treated with the growth factors delivered via a fibrin matrix. Two months after treatment, the coverage of the defects with mineralized bone as well as the volume of new bone formed was measured. As expected, delivering BMP-2 and PDGF-BB in wild-type mice enhanced regeneration to some extent. However, the regenerative effect of both growth factors was significantly enhanced in Il1r1^{-/-} mice, suggesting that IL-1R1 signalling impairs regeneration driven by BMP-2 and PDGF-BB (FIG. 11).

### IL-1R1 Signalling Inhibits the Response of Bone-Forming Cells to BMP-2 and PDGF-BB

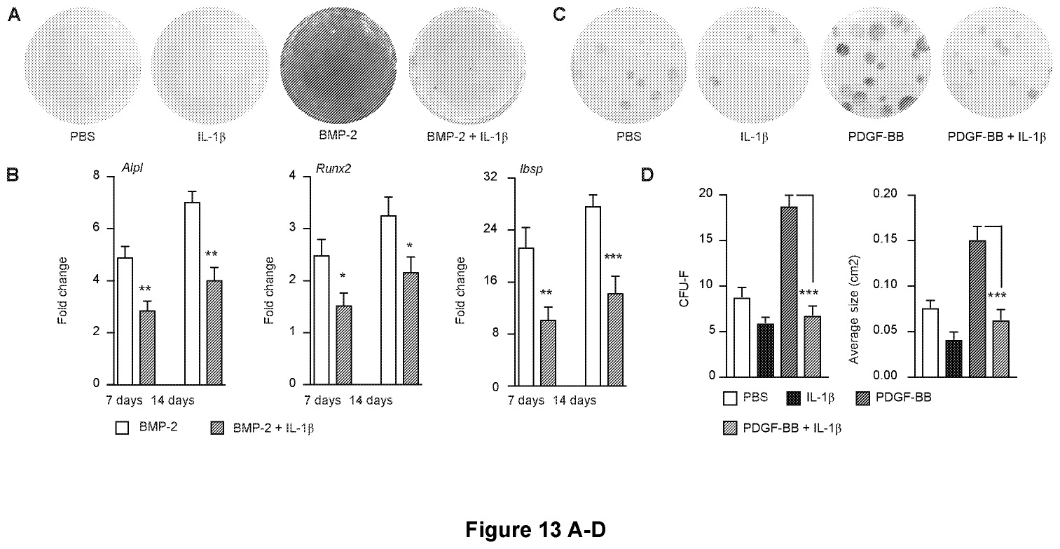

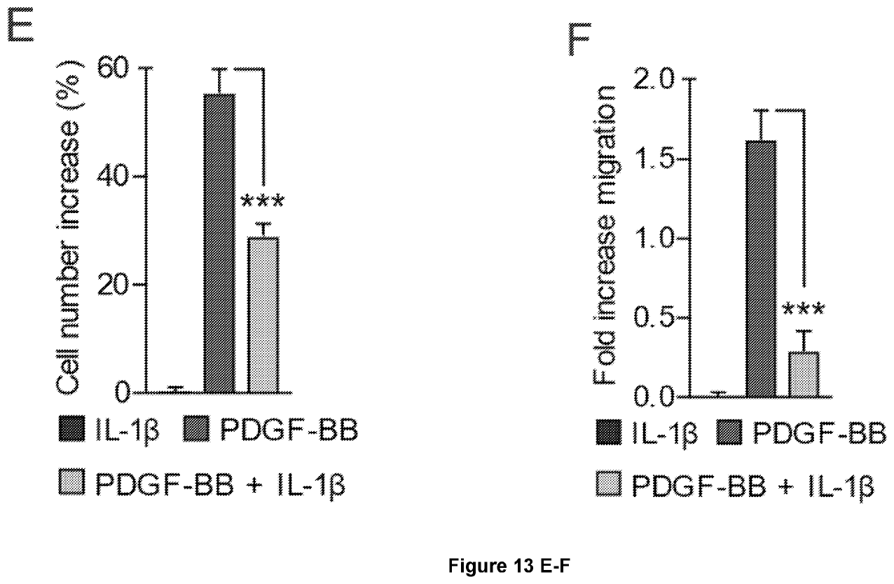

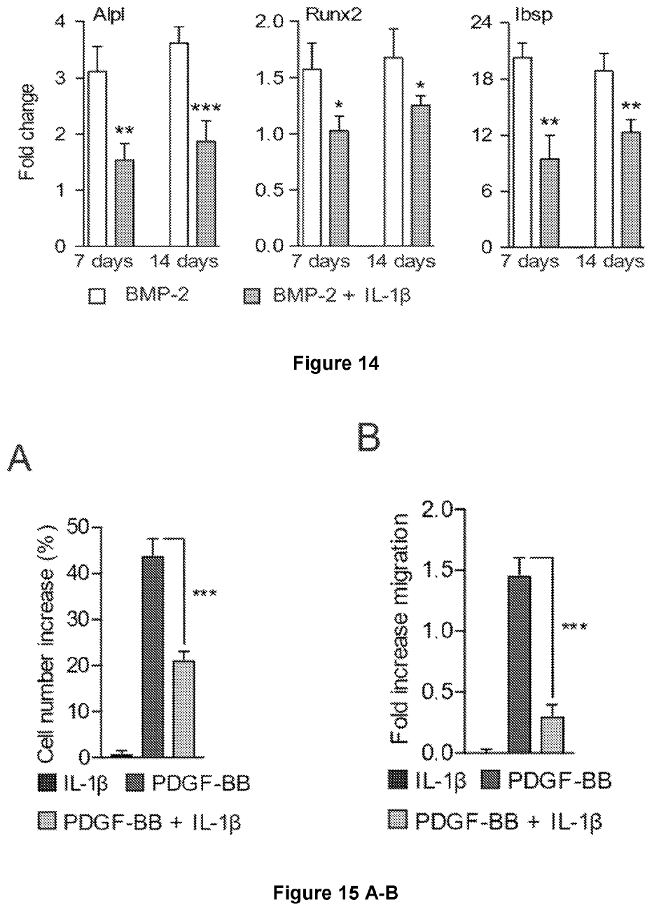

During bone regeneration, BMP-2 induces differentiation of bone-forming cells, i.e. skeletal stem/progenitor cells and osteoblasts, while PDGF-BB induces both migration and proliferation of these cells. Thus, we examined if IL-1β—the main IL-1R1 ligand released following bone injury—affects the ability of growth factors to induce these key cellular processes. As bone-forming cell models, we used compact bone-derived mesenchymal stromal cells (called MSCs herein—FIG. 12) and osteoblasts. IL-1β significantly inhibited the capacity of BMP-2 to upregulate osteoblast-specific genes and to induce matrix mineralization in MSCs (FIGS. 13 A and 13B). The same effect was observed with the expression of differentiation markers in osteoblasts (FIG. 14). Since MSCs highly express PDGF receptor-β (PDGFR-β or CD140b, FIG. 12), their stimulation with PDGF-BB enhances colony-forming unit-fibroblasts (CFU-F) formation. However, we found that IL-1β inhibits the boosting effect of PDGF-BB on CFU-F formation (FIGS. 13C and 13D). Similarly, IL-1β inhibited PDGF-BB-driven MSC and osteoblast proliferation and migration (FIGS. 13E and 13; FIG. 15), further supporting that IL-1R1 signalling impairs the response of these bone-forming cells to growth factors.

### Exposure to IL-1R1 Signalling Desensitizes MSCs and Osteoblasts to Growth Factors

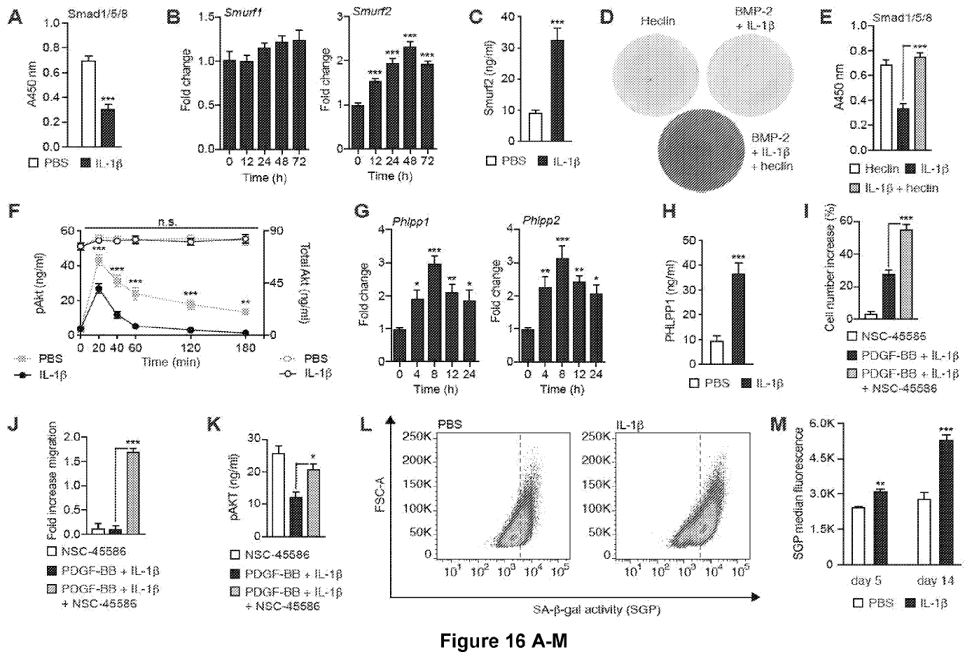

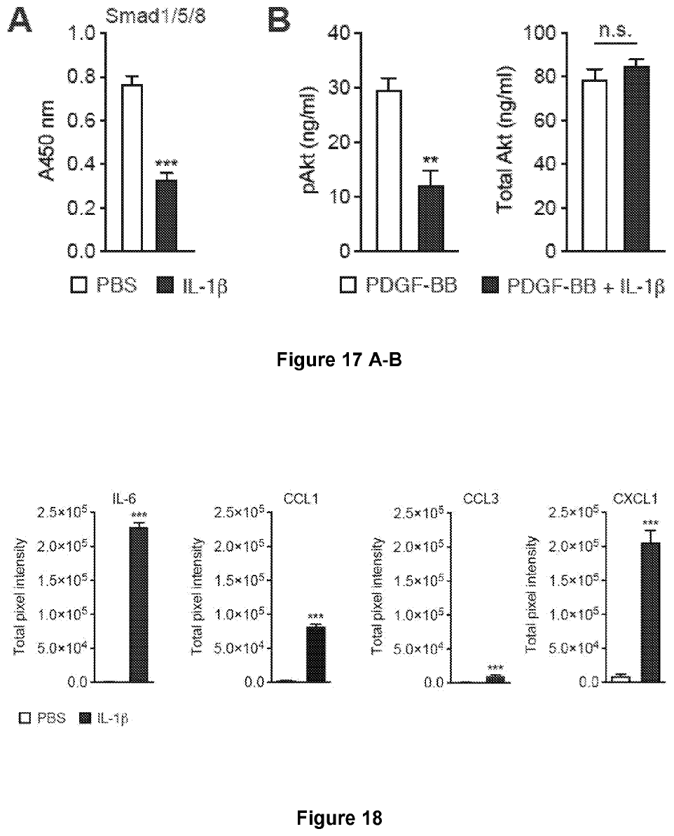

Because Smad proteins (Smad1/5/8) are critical in BMP-2 signalling transduction, we explored if IL-1R1 activation affects Smad1/5/8. Using an ELISA system specific for Smad1/5/8, we found that stimulation of MSCs and osteoblasts with IL-1β leads to a decrease of Smad1/5/8 (FIG. 16A, FIG. 17A). Moreover, IL-1β significantly upregulated the expression of the E3 ubiquitin-protein ligase Smurf2 (FIGS. 16B and 16C) which targets Smad1/5/8 for ubiquitination and degradation. To validate that IL-1β inhibits BMP-2-driven osteoblastic differentiation via Smad1/5/8 degradation by Smurf2, we used heclin—a selective inhibitor of Smurf2. Inhibition of BMP-2-driven osteoblastic differentiation by IL-1β was stopped in the presence of heclin as shown by a rescue of matrix mineralization (FIG. 16D). Further confirming the IL-1β-Smurf2-Smad1/5/8 axis, Smad1/5/8 degradation following IL-1β stimulation was inhibited in the presence of heclin (FIG. 16E).

Since Akt (protein kinase B) phosphorylation is central in PDGF-BB signalling, we tested if IL-1R1 stimulation affects this kinase. When MSCs and osteoblasts were pre-incubated with IL-1β, Akt phosphorylation which is normally induced after PDGF-BB stimulation was rapidly turned down (at Ser⁴⁷³) (FIG. 16F, FIG. 17B), indicating that Akt dephosphorylation is faster when cells are exposed to IL-1R1 signalling. Because Akt dephosphorylation is mainly driven by pleckstrin homology domain leucine-rich-repeats protein phosphatases (PHLPPs), we further tested if IL-1β modulates their expression. Remarkably, we found that MSC stimulation with IL-1β quickly increases the expression of PHLPPs (FIG. 16G, 16H). Then, to verify that IL-1β inhibits the response to PDGF-BB via PHLPP-driven dephosphorylation of Akt, we used NCS-45586 which is a selective inhibitor of PHLPPs). Inhibition of MSC proliferation and migration by IL-1 β as well as Akt dephosphorylation was suppressed in the presence of NCS-45586 (FIG. 16I, 16J, 16K), confirming the IL-16-PHLPPs-Akt pathway.

Last, we tested the effect of IL-1β on MSC senescence, because Smurf2 has been linked to this cellular process. We chose to measure senescence-associated β-galactosidase (SA-β-gal), as an increase of its activity is considered a hallmark for senescence. Interestingly, we found that treatment of MSCs with IL-16 increases SA-β-gal activity in a time-dependent manner, suggesting that IL-1β accelerates the induction of senescence in these cells (FIGS. 16L and 16M). In addition, MSC treatment with IL-16 induced the secretion of typical senescence-associated cytokines including IL-6, CC chemokine ligands 1 (CCL1) and 3 (CCL3), as well as CXC chemokine ligand 1 (CXCL1, IL-8 homologue in the mouse) (FIG. 18).

### Local Delivery of BMP-2 or PDGF-BB Induces the Release of IL-1β by Macrophages

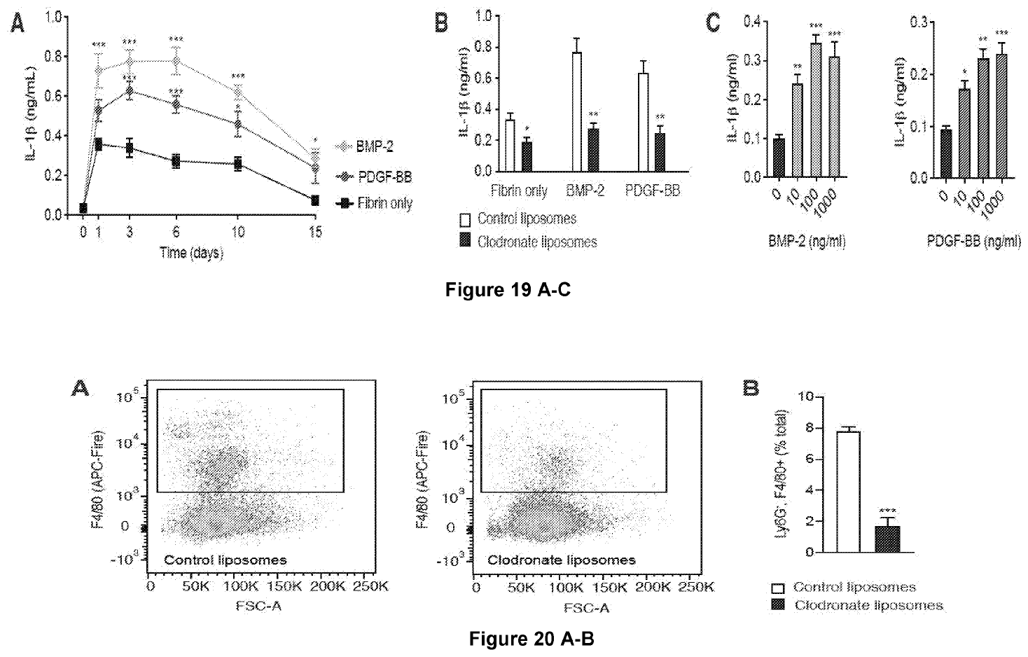

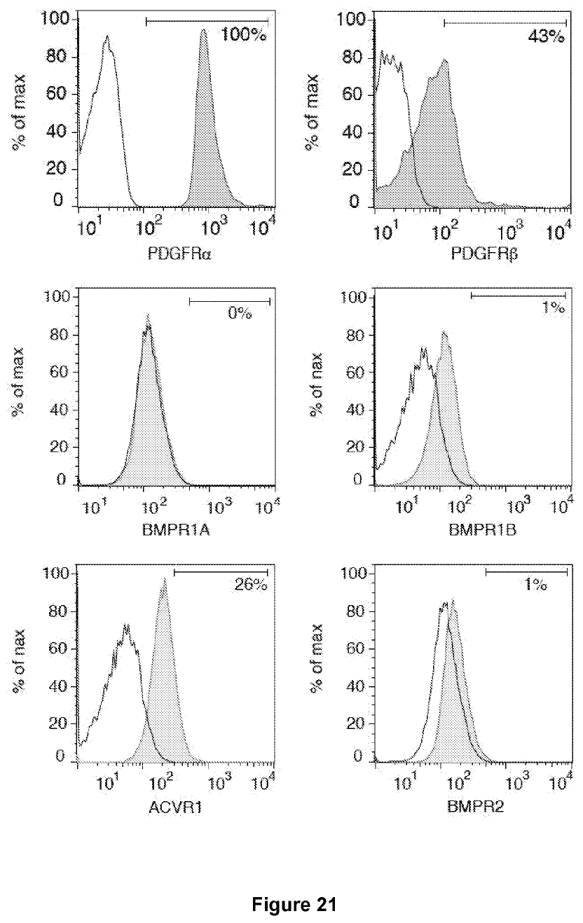

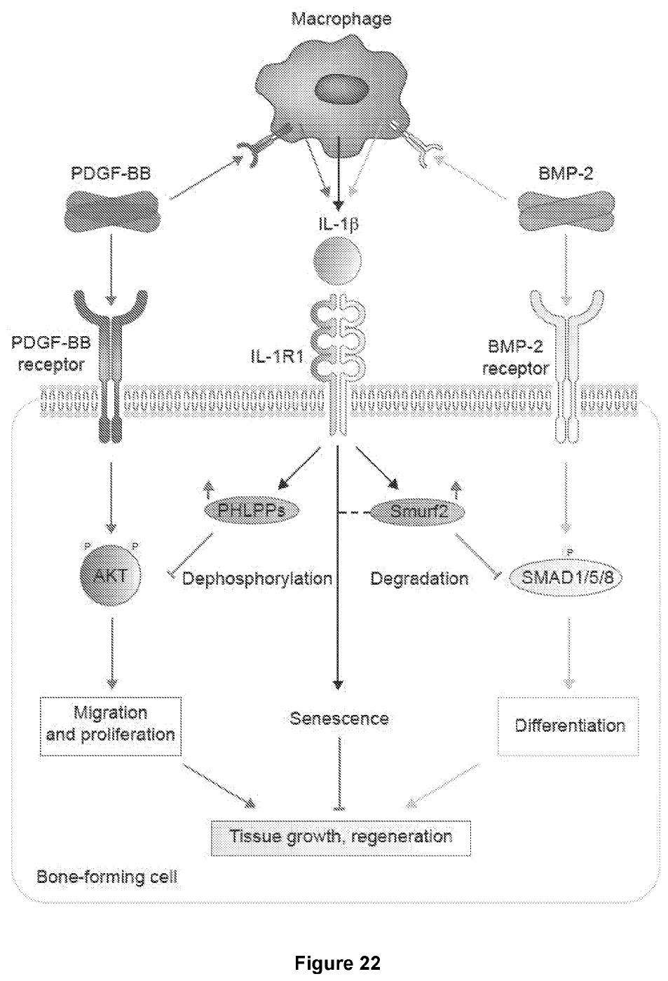

Because IL-1β is the main IL-1R1 ligand in the context of bone healing, we measured its concentration in calvarial defects following treatment with growth factors. Surprisingly, while IL-1β was released after bone injury, delivering BMP-2 or PDGF-BB led to a significant increase of the cytokine during the first two weeks following treatment FIG. 19A). Next, to determine which cell type was the primary source of IL-1β in the defect microenvironment, we repeated the experiment in mice where macrophages were depleted by clodronate liposomes (FIG. 20), since these cells release large amounts of IL-1β. Interestingly, in the absence of macrophages, IL-13 concentration in the bone defects was not enhanced by the delivery of BMP-2 or PDGF-BB, suggesting that the two growth factors trigger the release of IL-1β via macrophages (FIG. 19B). Indeed, we confirmed that primary bone marrow-derived macrophages (unpolarized) are equipped to respond to PDGF-BB and BMP-2 since they express some of their receptors (PDGFRα, PDGFRβ and ACVR1 as well as BMPR1B and BMPR2 to a lesser extent; FIG. 21). Confirming that the growth factors trigger the release of IL-1β by macrophages, in vitro stimulation of macrophages with BMP-2 or PDGF-BB induced the release of IL-1β (FIG. 19C). Taken together, we show that exposure to IL-1R1 signalling inhibits the response of bone-forming cells (MSCs and osteoblasts) to BMP-2 and PDGF-BB, while local delivery of the growth factors further promotes IL-1R1 signalling via triggering the release of IL-1β by macrophages (FIG. 22).

### Fusing PIGF₁₂₃₋₁₄₁ to PDGF-BB and IL-1Ra Confers Super-Affinity for ECM Proteins

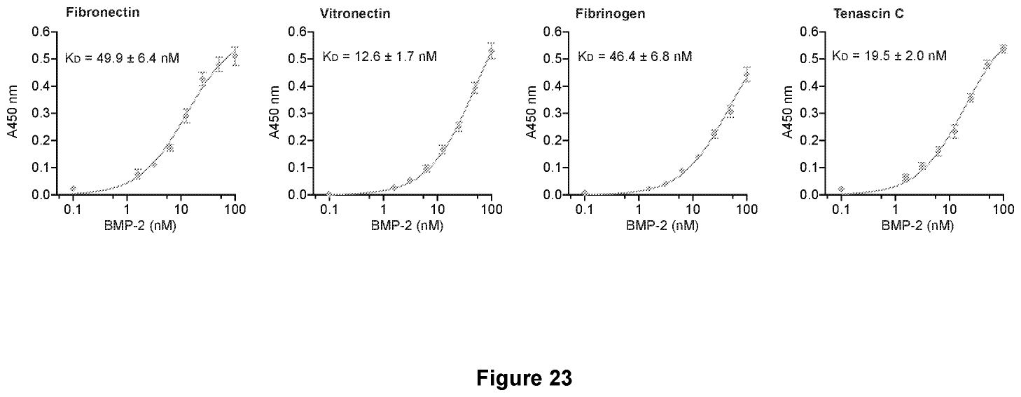

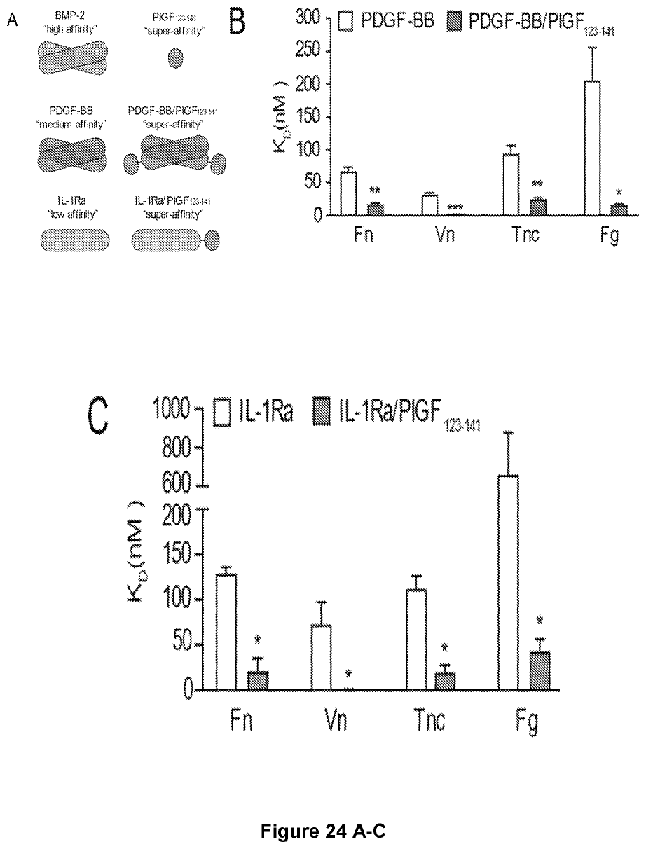

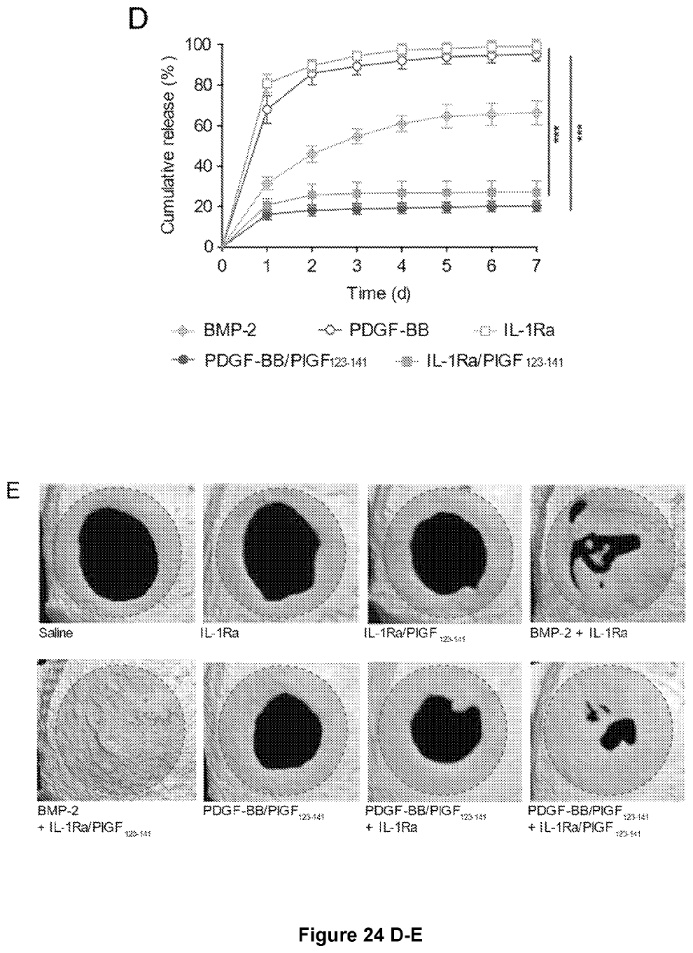

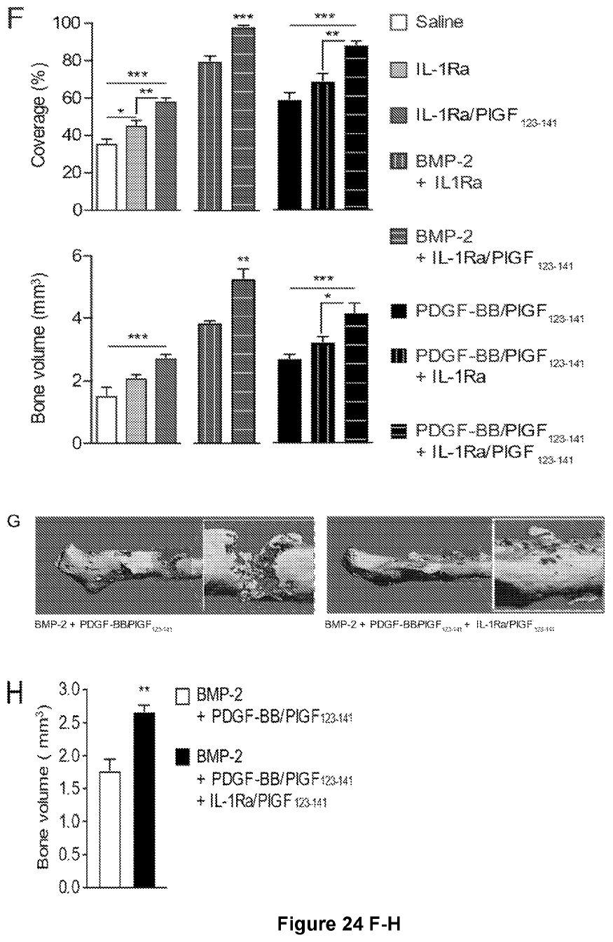

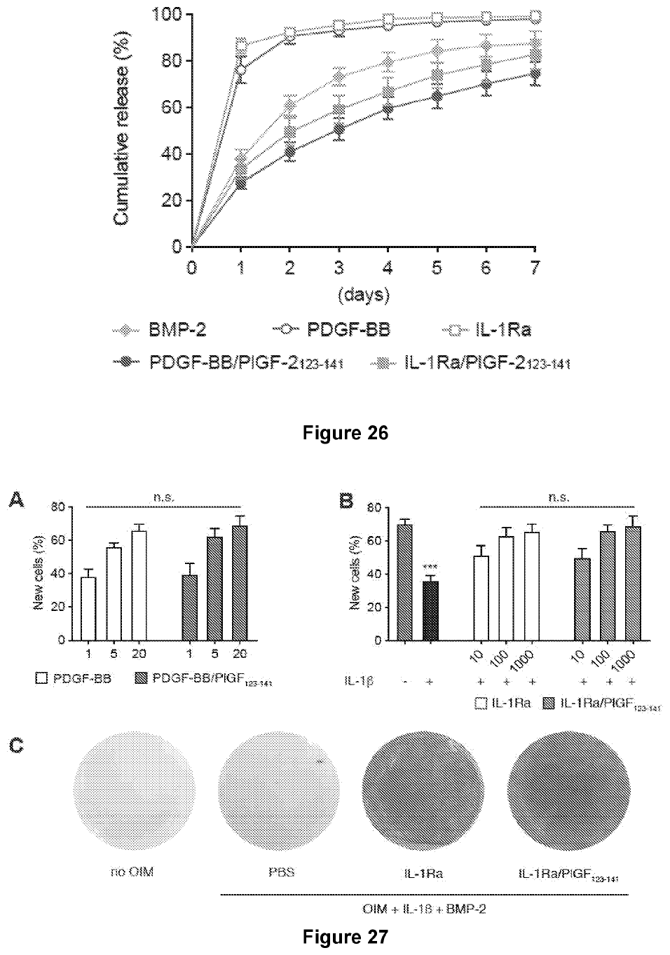

Since we found that IL-1R1 signalling rigorously inhibits the pro-regenerative effects of BMP-2 and PDGF-BB, we thought to co-deliver the growth factors with IL-1 receptor antagonist (IL-1Ra) to promote superior regeneration. First, to optimize delivery, we decided to enhance the affinity of the recombinant proteins to fibrin and endogenous extracellular matrix (ECM). BMP-2 is known to have a high binding affinity for fibrin and various ECM proteins and we confirmed that the growth factor strongly binds fibrin and some common ECM proteins (fibronectin, vitronectin, and tenascin C) (FIG. 23). The binding affinity of PDGF-BB for fibrin and other ECM proteins is known to be medium, while the ability of IL-1Ra to bind ECM proteins is poorly documented. Thus, we engineered PDGF-BB and IL-1Ra to contain a very high affinity ECM/fibrin-binding sequence derived from placenta growth factor (PIGF). PIGF₁₂₃₋₁₄₁ was added at the C-terminus of PDGF-BB and IL-1Ra to generate PDGF-BB/PIGF₁₂₃₋₁₄₁ and IL-1Ra/PIGF₁₂₃₋₁₄₁ (FIG. 24A, FIG. 25). Fusing PIGF₁₂₃₋₁₄₁ to PDGF-BB and IL-1Ra provided very strong binding (i.e. super-affinity) to all ECM proteins tested (fibronectin, vitronectin, tenascin C, and fibrinogen), with 4 to 100-fold increase in affinity (FIG. 24B, 24C). Moreover, BMP-2, as well as PIGF₁₂₃₋₁₄₁-fused PDGF-BB and IL-1Ra, were strongly retained in fibrin, while wild-type PDGF-BB and IL-1Ra were quickly released (FIG. 24D). Although BMP-2, PDGF-BB/PIGF₁₂₃₋₁₄₁, and IL-1Ra/PIGF₁₂₃₋₁₄₁ were retained in fibrin, they were gradually released in the presence of the protease plasmin which cleaves fibrin fibers and PIGF₁₂₃₋₁₄₁ (FIG. 26). Notably, fusing PIGF₁₂₃₋₁₄₁ to PDGF-BB did not alter its activity of the growth factor, since MSC proliferation in response to wild-type PDGF-BB and PDGF-BB/PIGF₁₂₃₋₁₄₁ was similar (FIG. 27). Likewise, IL-1Ra was not compromised by the fusion with PIGF₁₂₃₋₁₄₁, since wild-type IL-1Ra and IL-1Ra/PIGF₁₂₃₋₁₄₁ displayed the same inhibitory activity (FIG. 27).

### Super-Affinity IL-1Ra Augments Growth Factor-Driven Bone Regeneration

To test if local delivery of super-affinity IL-1Ra supports bone regeneration driven by BMP-2 and PDGF-BB, we first used the critical-size calvarial defect model in the mouse. Delivering wild-type IL-1Ra without growth factors slightly enhanced regeneration compared to fibrin only control and IL-1Ra/PIGF₁₂₃₋₁₄₁ induced significantly more bone formation compared to wild-type IL-1Ra (FIGS. 24E and 24F). Co-delivering BMP-2 with wild-type IL-1Ra strongly stimulated bone regeneration compared to delivering BMP-2 alone (FIG. 11; FIGS. 24E and 24F). However, the co-delivery of BMP-2 with IL-1Ra/PIGF₁₂₃₋₁₄₁ promoted significantly more bone formation compared to co-delivering BMP-2 with wild-type IL-1Ra, leading to nearly 100% coverage of the defect and larger bone volume formation (FIG. 24E and FIG. 24F). Similarly, the co-delivery of PDGF-BB/PIGF₁₂₃₋₁₄₁ with IL-1Ra/PIGF₁₂₃₋₁₄₁ significantly improved regeneration compared to the delivery of PDGF-BB/PIGF₁₂₃₋₁₄₁ alone and the co-delivery of PDGF-BB/PIGF₁₂₃₋₁₄₁ with wild-type IL-1Ra (FIGS. 24E and 24F).

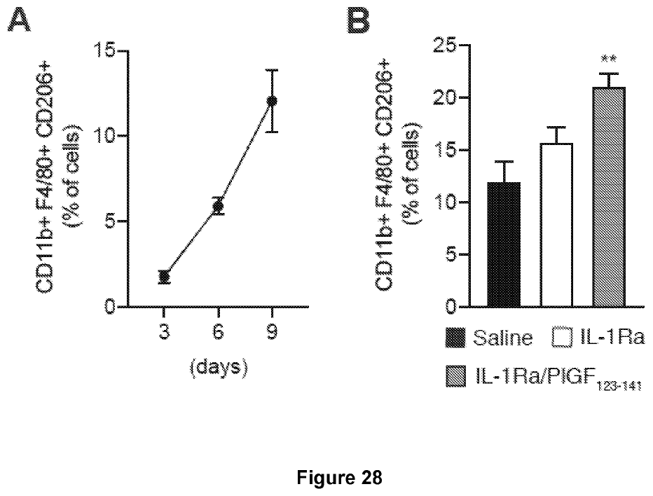

Because IL-1Ra inhibits the pro-inflammatory effect of IL-1β, we then tested whether macrophage polarization during bone regeneration was influenced by the delivery of the antagonist. We focused on the upregulation of the mannose receptor CD206 since this surface marker is well accepted for detecting M2-like macrophages in tissues. In defect treated with fibrin only, the population of CD206 positive macrophages gradually increased during the first 9 days post-injury (FIG. 28). Thus, we selected day 9 for further analysis. Interestingly, mice treated with IL-1Ra/PIGF₁₂₃₋₁₄₁ displayed a higher percentage of CD206 positive macrophages after 9 days (FIG. 28), suggesting that IL-1Ra/PIGF₁₂₃₋₁₄₁ promotes faster polarization of macrophages towards an anti-inflammatory phenotype.

Last, we tested if super-affinity IL-1Ra could improve bone regeneration driven by growth factors in a critical-size defect model in the mouse femur. Defect of 2 mm were generated and stabilized with a metallic plate. Then, BMP-2 and PDGF-BB/PIGF₁₂₃₋₁₄₁ were delivered with or without IL-1Ra/PIGF₁₂₃₋₁₄₁ via fibrin. Remarkably, co-delivering both growth factors with IL-1Ra/PIGF₁₂₃₋₁₄₁ significantly induced the formation of more bone volume, compared to delivering the growth factors without IL-1Ra/PIGF₁₂₃₋₁₄₁ leading to nearly full regeneration of the defects after 3 months (FIGS. 24G and 24H).

### Discussion

Growth factors are powerful tools for regenerative medicine, but their application has been limited by many issues that could probably be overcome by a better understanding of their signalling dynamics and by optimizing delivery systems. In addition, the response of immune cells to recombinant growth factor delivery is largely elusive but likely very important for optimizing growth factor effectiveness and safety. Actually, the immune system is probably a critical parameter to consider when designing regenerative strategies based on growth factors. Here, we have hypothesized that the regenerative response to growth factors is influenced by the immune and inflammatory microenvironment which practically always accompanies tissue repair and regeneration. Because the pro-inflammatory cytokine IL-1 is a central mediator of inflammation and immunity, we explored the extent to which IL-1R1 signalling affects regeneration driven by growth factors and we focused on bone as a regeneration system.

Among models commonly used for evaluating bone regeneration, we first chose the critical-size calvarial defect model in the mouse for its reliability and low variability in both basic and applied research. In addition, this model is ideal to investigate the impact of IL-1R1 signalling during growth factor-driven regeneration, due to the availability of the IL-1R1 deficient mouse. Remarkably, we found that the regenerative effect of recombinant BMP-2 and PDGF-BB is greatly enhanced in IL1r1^{-/-} mice, strongly indicating that IL-1R1 signalling impairs the capacity of the growth factors to promote regeneration. Most importantly, it suggests that blocking IL-1R1 could be used to improve regeneration driven by the BMP-2 and PDGF-BB.

In the context of bone regeneration, BMP-2 and PDGF-BB are well-known to act on bone-resident MSCs and osteoblasts to promote new bone formation. BMP-2 promotes differentiation, while PDGF-BB promotes chemotaxis and proliferation. Here, we found that IL-1R1 signalling inhibits the fundamental morphogenic effects triggered by both growth factors. Mechanistically, we propose that exposure to IL-1R1 signalling makes MSCs and osteoblasts less responsive to BMP-2 and PDGF-BB, by two mechanisms. In the context of BMP-2, IL-1R1 signalling increases Smurf2 expression and promotes Smad1/5/8 degradation, which results in an impairment of BMP-2-driven differentiation, due to lower Smad1/5/8 levels. In line with this finding, it has been shown that NF-κB—the main transcription factor activated by IL-1R1 signalling—inhibits osteogenic differentiation of MSCs by promoting β-catenin degradation via Smurf2. In the context of PDGF-BB, IL-1R1 signalling increases expression of PHLPPs which drives quicker Akt dephosphorylation and impairs proliferation and migration responses which are normally induced by PDGF-BB. Similarly, it has been shown that inflammatory mediators signalling via NF-κB also enhance PHLPP1 in human chondrocytes. Notably, because Smad1/5/8 and Akt are critical in the signalling of many growth factors, activation of IL-1R1 may not only inhibit the activity of recombinant BMP-2 and PDGF-BB, but also several other potential therapeutics such as BMPs and growth factors in the vascular, fibroblast, and epidermal growth factors families.

The senescence of cells critical for repair and regeneration, such as stem cells and progenitor cells, is a major hurdle for the healing of musculoskeletal tissues. Recently, NF-κB has been linked to skeletal stem/progenitor cell senescence and dysfunction in human and mouse. Here, in addition to the negative effects of IL-1R1 signalling in growth factor signalling, we show that long-term activation (1-2 weeks) of IL-1R1 accelerates MSC senescence, as SA-β-gal activity in MSCs was enhanced over time when cells were treated with IL-1β. While the exact signalling mechanisms involved in IL-1R1-driven senescence are still unclear, the enhanced expression of Smurf2 following IL-1R1 activation is probably important, since it has been shown that Smurf2 activity is strongly linked with cellular senescence.

Interestingly, we found that the delivery of BMP-2 and PDGF-BB in bone defects significantly increases IL-1β concentration in the injured tissue. The increase of IL-1β is likely due to macrophage response to BMP-2 and PDGF-BB since IL-1β concentration was not enhanced by delivering the growth factors in mice depleted of monocytes and macrophages. Thus, the detrimental effect of IL-1R1 signalling in MSCs and osteoblasts is likely exacerbated in vivo by macrophages responding to the delivery of recombinant BMP-2 and PDGF-BB. The effects of BMP-2 and PDGF-BB on monocytes and macrophages are still unclear. For instance, it has been shown that BMP-2 and PDGF-BB induce monocyte and macrophage chemotaxis. In addition, BMP-2 may modulate the polarization of macrophages towards an anti-inflammatory phenotype either positively or negatively. Nevertheless, one of the major side effects of BMP-2 in clinical use is rampant inflammation. Here, our data suggest that macrophage response to BMP-2 and PDGF-BB triggers the release of IL-1β, which encourages inflammation.

Because IL-1β inhibits the pro-regenerative effects of BMP-2 and PDGF-BB and since both growth factors further trigger IL-1β release by macrophages, we thought of co-delivering them with IL-1Ra to enhance bone regeneration. Recombinant BMP-2 and PDGF-BB are both USFDA-approved to promote bone formation. However, BMP-2 and PDGF-BB have raised major concerns regarding safety and cost-effectiveness for multiple clinical applications, likely due to the use of high doses coupled with sub-optimal delivery systems. IL-1Ra (Anakinra, Kineret) is approved for the treatment of rheumatoid arthritis and neonatal-onset multisystem inflammatory disease. Yet, IL-1Ra needs to be used at very high doses (>100 mg per injection) with multiple bulk administrations and the use of this immunosuppressant has been reported to lead to infections and immunogenicity. Thus, considering the overall clinical efficacy of recombinant BMP-2, PDGF-BB, and IL-1Ra, better delivery systems need to be developed to allow precise localization and retention of low doses if the drugs at the delivery sites. One of the strategies is to engineer recombinant proteins to strongly bind a biomaterial carrier and endogenous ECM present in the tissue where they are delivered. Because PDGF-BB and IL-1Ra naturally have a relatively low affinity for ECM components and fibrin, we engineered them to include PIGF₁₂₃₋₁₄₁, while we decided to use BMP-2 in its wild-type form as it naturally binds many ubiquitous ECM proteins with high affinity. This strategy allows super-affinity PDGF-BB and IL-1Ra as well as BMP-2 to be retained in a fibrin matrix and released “on-demand” via proteases which naturally cleave both fibrin fibers and the PIGF₁₂₃₋₁₄₁ sequence.

To test all treatment combinations, we first used the mouse calvarial defect as a screening model, due to its simplicity and reliability for evaluating bone regenerative strategies. Then, to test the most significant treatments, we moved to a mouse femur critical-size defect model for its higher clinical relevance. Delivering low doses of wild-type BMP-2 (<1 mg per defect) without a particular delivery system or osteo-inductive biomaterial is known to have a modest regenerative effect in calvarial defect, while low dose of wild-type PDGF-BB (<1 mg per defect) usually has no significant effect. As expected, we observed that wild-type BMP-2 triggers regeneration to some extent but we found that the super-affinity versions of PDGF-BB and IL-1Ra have greater regenerative capacity compared to their wild-type forms. The enhanced activity of engineered PDGF-BB and IL-1Ra is likely due to their much higher binding-affinity to fibrin and endogenous ECM proteins and therefore higher retention at the delivery site. Most importantly, we demonstrated that co-delivering BMP-2 with super-affinity IL-1Ra or co-delivering super-affinity PDGF-BB with super-affinity IL-1Ra significantly stimulates superior bone regeneration compared to the delivery of BMP-2 or super-affinity PDGF-BB alone. Furthermore, we confirmed that the regenerative capacity of the growth factors is enhanced by super-affinity IL-1Ra in the femur defect model. Thus, the results in both models clearly show that inhibiting IL-1R1 signalling via a super-affinity IL-1Ra enhances the bone regenerative response to BMP-2 and PDGF-BB.

Macrophage polarization from an inflammatory to an anti-inflammatory state is well-known to be important for tissue healing. Therefore, we also tested if the delivery of IL-1Ra affects macrophage polarization. Interestingly, mice treated with super-affinity IL-1 Ra displayed a higher percentage of anti-inflammatory-like macrophages which are commonly characterized by the surface expression of CD206. This suggests that, in addition to restoring BMP-2 and PDGF-BB signalling in MSCs and osteoblasts, super-affinity IL-1Ra may also promote bone regeneration by supporting macrophages polarization towards an anti-inflammatory phenotype.

The strategy of locally inhibiting IL-1R1 with IL-1Ra/PIGF₁₂₃₋₁₄₁ to support the regenerative activity of BMP-2 or PDGF-BB shows very promising results in murine models. However, before translation to the clinic, such a system needs to be validated in larger animals such as the sheep which presents a bone structure more similar to that of humans. The system may also be evaluated in other models of bone formation such as spinal (for BMP-2) and foot/ankle (for PDGF-BB) fusion. Nevertheless, because the wild-type form of the growth factors, and IL-1Ra have been clinically approved, translation of this strategy could be facilitated.

In conclusion, we highlight the importance of considering inflammation and the immune system when using growth factors for regenerative medicine applications. Indeed, integrating a control of immune pathways in regenerative therapies based on growth factors could be as important as having smart delivery systems that control growth factor localization and release. In the context of bone regeneration, we demonstrate that an engineered matrix-binding form of IL-1Ra considerably improves the regenerative efficiency of recombinant BMP-2 and PDGF-BB. This strategy may not only be used for bone regeneration and formation but also in chronic bone inflammatory conditions or in other tissues where IL-1R1 signalling has a negative impact.

### Example 4—Identification and Testing of Further ECM Binding Sequences to be Used in IL-1Ra Fusion Proteins

Identification of ECM-binding candidates

The PIGF-2₁₂₃₋₁₄₁ sequence is characterized by six blocks of one to four repetitions of R and K residues. Therefore, a protein motif containing six stretches of basic amino acids separated by one or two non-basic amino acids was generated and used to search the UniProtKB databank for proteins containing this search motif using the ScanProsite online tool described in E. de Castro et al., (2006) Nucleic Acids Res., vol. 34, no. Web Server issue, pp. W362-5.

The search motif was generated using the following rules: R and K are interchangeable and noted [RK].

A block of a single repetition of [RK] in PIGF-2₁₂₃₋₁₄₁ can translate to one or two repetitions noted [RK](1,2) in the search motif.

A block of n repetitions of [RK] in PIGF-2₁₂₃₋₁₄₁ can translate to a block of 2 to n+1 repetitions noted [RK](2, n+1) with n≥2.

Residues separating the blocks of [RK] in PIGF-2₁₂₃₋₁₄₁ can translate to one or two repetitions of any residue noted X in the search motif.

Human proteins between 50 and 1000 amino acids-long containing the resulting search motif were then identified. Growth factors from that list were then aligned against PIGF-2₁₂₃₋₁₄₁ using a basic local alignment search tool (BLAST) to generate an identity score using the method described in Altschul, S. F. et al., (1990), J. Mol. Biol., vol 215, no. 3, pp 403-10.

### [RK](2,4)X(1,2)[RK](1,2)X(1,2)-[RK](1,2)X(1,2)[RK](2,5)X(1,2)[RK](1,2)X(1,2)[RK](1,2)

The search identified 357 sequences containing the search motif, including four from growth factors as shown in Table 2A.

TABLE 2A
Results of motif search.

| Protein | Motif match |
|---|---|
| PIGF-2 | 123 RRRPKGRGKRRREKQR 138 |
| | (SEQ ID NO: 64) |
| NRTN | 146 RRLRQRRRLRRERVR 160 |
| | (SEQ ID NO: 63) |
| AREG | 126 RKKKGGKNGKNRRNRKKK 143 |
| | (SEQ ID NO: 51) |
| VEGF-A | 133 KKDRARQEKKSVRGK 147 |
| | (SEQ ID NO: 65) |

While two sequences are contained in PIGF-2 and vascular endothelial growth factor A (VEGF-A) from which the ECM-binding capacity is already known from Martino, M. M. et al, (2014) Science, vol. 343, no. 6173, pp. 885-8, the remaining two originate from neurturin (NRTN) and amphiregulin (AREG) and the affinity of these proteins for the ECM has yet to be characterized. NRTN₁₄₆₋₁₆₀, AREG₁₂₆₋₁₄₃, and VEGF-A₁₃₃₋₁₄₇ were aligned with PIGF-2₁₂₃₋₁₄₁ using BLAST to generate an identity score (Table 2B). Interestingly, NRTN₁₄₆₋₁₆₀ produced the highest score whereas the algorithm could not generate an alignment for VEGF-A₁₃₃₋₁₄₇.

TABLE 2B
Local alignment scores.

| Protein | Alignment | Score |
|---|---|---|
| NRTN | Query 1 RRRPKGRGKRRREKQR 16 | 14.6 |
| | (SEQ ID NO: 64) | |
| | Sbjct 6 RRRL------RRERVR 15 | |
| AREG | Query 8 GKRRR 12 | 12.9 |
| | Sbjct 9 GKNRR 13 | |
| VEGF-A | No alignment found | 0 |

### Binding Affinity of NRTN and AREG to ECM Proteins.

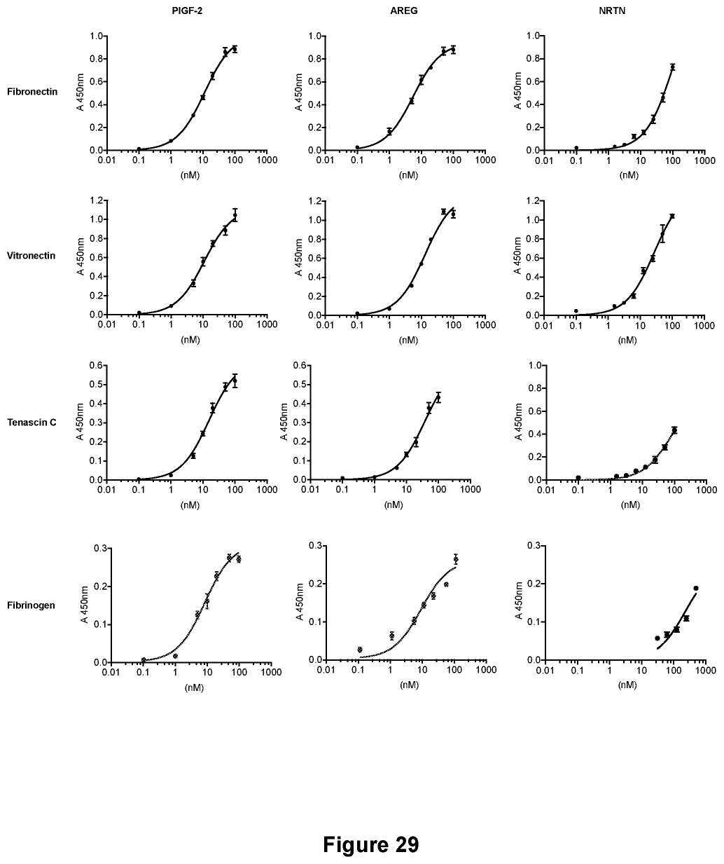

In order to confirm the ability of NRTN and AREG to bind the ECM, we tested their affinity for fibronectin, vitronectin, tenascin C, and fibrinogen (FIG. 29). Table 3 shows the K_{D} values of PIGF-2, NRTN and AREG for ECM proteins (n=3, mean±SEM). AREG showed a very high affinity for the ECM proteins, similar to that of PIGF-2, whereas NRTN showed a much lower affinity. This is surprising as NRTN shares a greater similarity with PIGF-2₁₂₃₋₁₄₁ than AREG does, as shown by their BLAST identity scores which would suggest that NRTN would have the highest affinity of the two newly identified growth factors.

TABLE 3
Binding affinity of PIGF-2, NRTN and AREG to ECM proteins.

| ECM | K_{D} ± SEM (nM) | | |
|---|---|---|---|
| PROTEINS | PIGF-2 | AREG | NRTN |
| Fibronectin | 11.6 ± 3.1 | 8.6 ± 3.8 | 50.15 ± 4.27 |
| Vitronectin | 13.9 ± 4.7 | 13.8 ± 1.6 | 27.59 ± 2.82 |
| Fibrinogen | 9.9 ± 3.8 | 8.2 ± 0.4 | >500 |
| Tenascin C | 16.6 ± 0.7 | 36.8 ± 0.3 | |

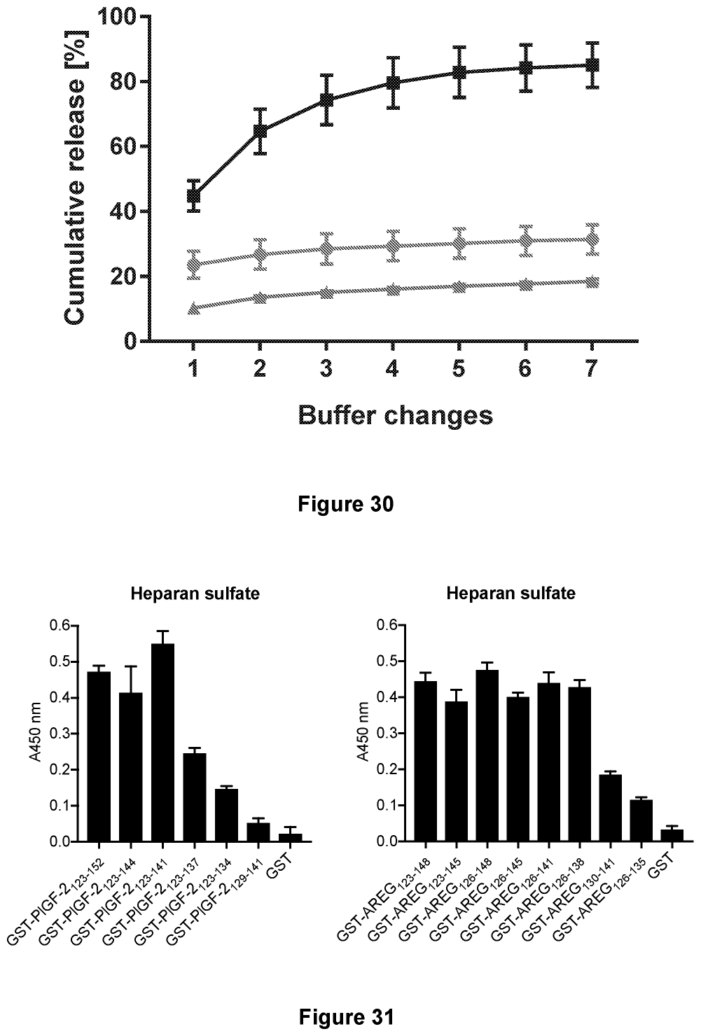

We then evaluated the ability of AREG to be retained in an ECM analogue. AREG, PIGF-2, and PIGF-1, whose affinity for the ECM is low, where loaded in fibrin matrix and the fraction of the protein dose released from the matrix was evaluated daily for seven days (FIG. 30). Over 60% PIGF-1 was released after two days while less than 30% of PIGF-2 and AREG were released after 7 days (n=3, mean±SEM). Remarkably, AREG was retained in the fibrin matrix in similar way as PIGF-2, with the majority of the proteins still bound to the matrix after seven days while over 50% of PIGF-1 was released after two days.

### NRTN and AREG Fragments Specifically Bind ECM Proteins and Heparan Sulphate.

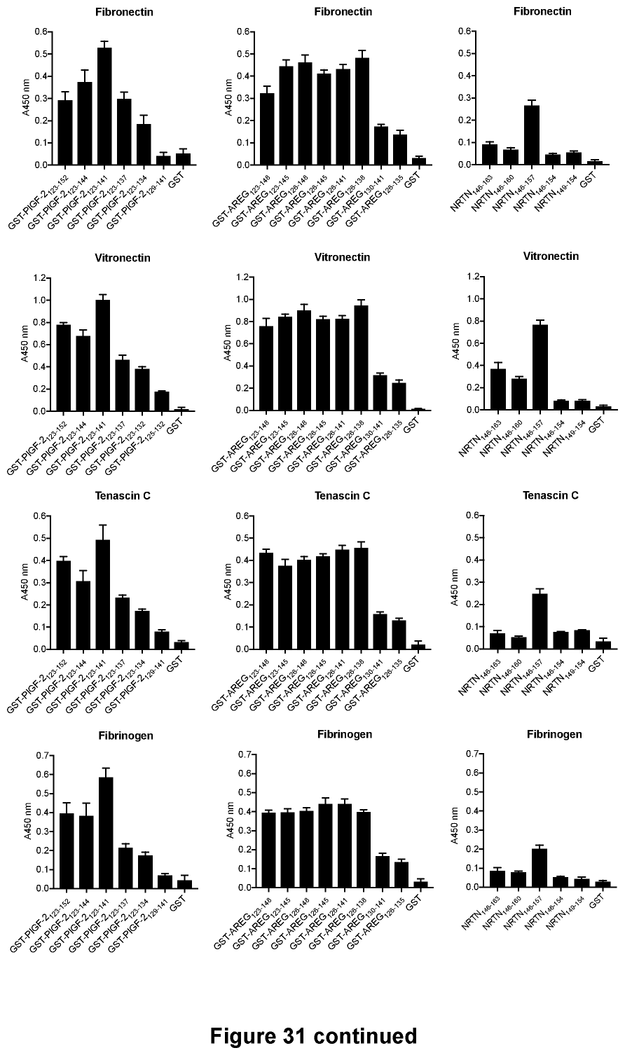

To identify the minimal ECM-binding sequence from NRTN and AREG we produced five and eight truncated constructs of NRTN₁₄₆₋₁₆₃ and AREG₁₂₃₋₁₄₈ (Table 4), respectively. Fragments were synthetized and cloned into the bacterial expression vector pGEX6P-1. The fragments were expressed in E. coli BL21 and purified with a GSTrap HP 5 ml and His-tag HP 5 ml (GE healthcare) affinity columns. Their affinity for fibronectin, vitronectin, tenascin C, and fibrinogen, as well as heparan sulfate was tested and the results are shown in FIG. 31.

TABLE 4
ECM-binding sequence constructs derived
from NRTN and AREG.

| Name of | |
|---|---|
| fragments | Sequence of fragments |
| NRTN₁₄₆₋₁₆₃ | RRLRQRRRLRRERVRAQP |
| | (SEQ ID NO: 62) |
| NRTN₁₄₆₋₁₆₀ | RRLRQRRRLRRERVR--- |
| | (SEQ ID NO: 63) |
| NRTN₁₄₆₋₁₅₇ | RRLRQRRRLRRE------ |
| | (SEQ ID NO: 3) |
| NRTN₁₄₆₋₁₅₄ | RRLRQRRRL--------- |
| | (SEQ ID NO: 66) |
| NRTN₁₄₉₋₁₅₄ | ---RQRRRL--------- |
| | (SEQ ID NO: 67) |
| AREG₁₂₃₋₁₄₈ | KPKRKKKGGKNGKNRRNRKKKNPCNA |
| | (SEQ ID NO: 68) |
| AREG₁₂₃₋₁₄₅ | KPKRKKKGGKNGKNRRNRKKKNP--- |
| | (SEQ ID NO: 57) |
| AREG₁₂₆₋₁₄₈ | ---RKKKGGKNGKNRRNRKKKNPCNA |
| | (SEQ ID NO: 56) |
| AREG₁₂₆₋₁₄₅ | ---RKKKGGKNGKNRRNRKKKNP--- |
| | (SEQ ID NO: 53) |
| AREG₁₂₆₋₁₄₁ | ---RKKKGGKNGKNRRNRK---- |
| | (SEQ ID NO: 49) |
| AREG₁₂₆₋₁₃₈ | ---RKKKGGKNGKNRR------- |
| | (SEQ ID NO: 2) |
| AREG₁₃₀₋₁₄₁ | -------GGKNGKNRRNRK---- |
| | (SEQ ID NO: 69) |
| AREG₁₂₆₋₁₃₅ | ---RKKKGGKNGK---------- |
| | (SEQ ID NO: 70) |

PIGF-2₁₂₃₋₁₄₁ (RRRPKGRGKRRREKQRPTD), NRTN₁₄₆₋₁₅₇ (RRLRQRRRLRRE) and AREG₁₂₆₋₁₃₈ (RKKKGGKNGKNRR) displayed a higher affinity for both ECM proteins and heparan sulphate. The results obtained by ELISA assay (n=3, mean±SEM). The summary of results is shown in Table 5. Fusion proteins comprising these sequences fused to IL-1Ra are expected to have the same or similar activity to fusion proteins comprising an ECM binding peptide from PIGF fused to IL-1Ra. In addition, ECM-binding sequences comprising of consisting of NRTN₁₄₆₋₁₅₇ or AREG₁₂₆₋₁₃₈ may be used to deliver proteins other than IL-1Ra to the ECM, such proteins including other biological agents such as growth factors, cytokines, antibodies and the like, particularly for wound healing and tissue regeneration.

TABLE 5
Minimal ECM-binding sequence from
NRTN and AREG

| Minimal sequences | |
|---|---|
| name | Sequences |
| NRTN₁₄₆₋₁₅₇ | RRLRQRRRLRRE |
| | (SEQ ID NO: 3) |
| AREG₁₂₆₋₁₃₈ | RKKKGGKNGKNRR |
| | (SEQ ID NO: 2) |

### Example 5: Binding of AREG₁₂₆₋₁₃₈/PDGF-BB Fusion Protein to ECM Proteins

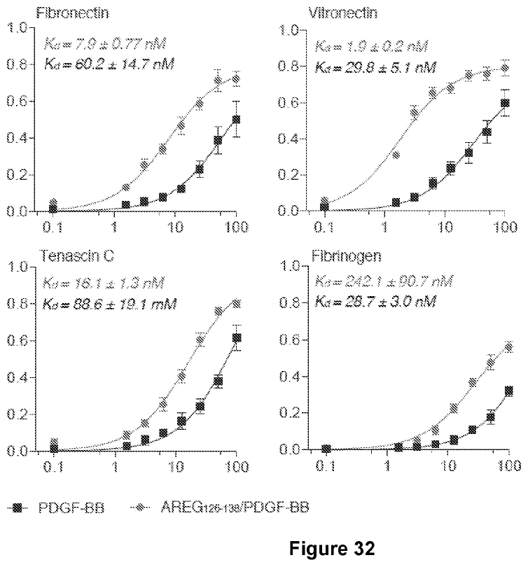

AREG₁₂₆₋₁₃₈ was fused at the N-terminus of PDGF-BB to generate AREG₁₂₆₋₁₃₈/PDGF-BB. ELISA plate wells were coated with ECM proteins (fibronectin, vitronectin, tenascin C, and fibrinogen) and further incubated with AREG₁₂₆₋₁₃₈-fused or wild-type proteins. Graphs show signals given by an antibody detecting PDGF-BB. The signals were fitted by non-linear regression to obtain the dissociation constant (K_{d}) using A450 nm=Bmax*[protein]/(K_{d}+[protein]) where [protein] is the concentration of PDGF-BB or AREG₁₂₆₋₁₃₈/PDGF-BB. Representative binding curves are shown in FIG. 32.

The AREG₁₂₆₋₁₃₈-fused protein displayed greater affinity for the ECM protein tested (fibronectin, vitronectin, tenascin C, and fibrinogen) than the wild-type protein.

### Example 6: AREG₁₂₆₋₁₃₈/PDGF-BB Fusion Protein for Treating Diabetic Wounds

To evaluate whether localized delivery of PDGF-BB variants promotes healing of diabetic wounds, we utilized the Lepr^{db/db} mouse again. A single low dose of PDGF-BB or the fusion protein was delivered topically in saline following full-thickness wounding (0.5 μg of PDGF-BB, and an equimolar dose of the engineered form AREG₁₂₆₋₁₃₈/PDGF-BB).

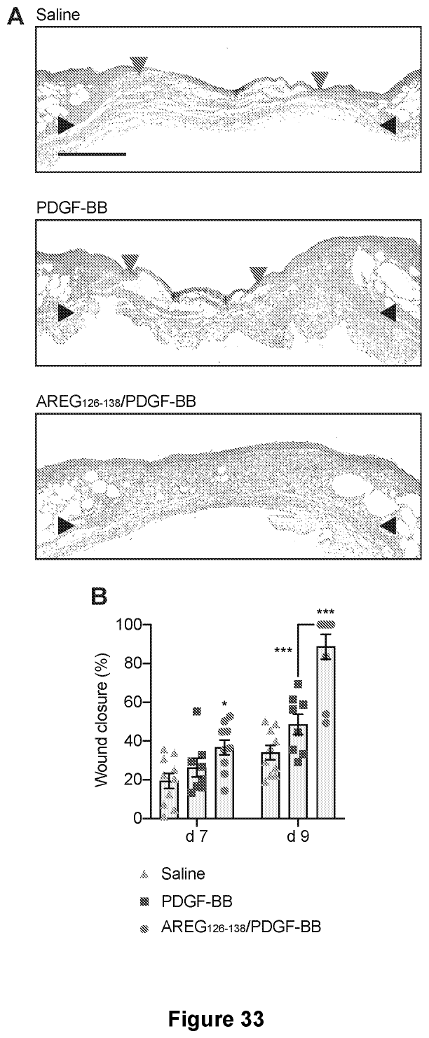

FIG. 33 A shows representative histology (hematoxylin and eosin staining) 7 or 9 d post-treatment. Black arrows indicate wound edges and gray arrows indicate tips of epithelium tongue. The epithelium (if any) appears as a homogeneous keratinocyte layer on top of the wounds. The granulation tissue under the epithelium contains granulocytes with dark nuclei. Fat tissue appears as transparent bubbles. Scale bar=1. FIG. 33 B shows wound closure following treatment with PDGF-BB variants evaluated by histomorphometric analysis of tissue sections. Data are means±SEM, n=10 wounds for saline and AREG₁₂₆₋₁₃₈/PDGF-BB, n=8 wounds for PDGF-BB. One-way ANOVA with Bonferroni post hoc test for pair-wise comparisons (significances shown are between saline and the other groups, unless indicated otherwise). *P≤0.05, ***P≤0.001.

One week after treatment, wounds that received AREG₁₂₆₋₁₃₈/PDGF-BB showed significantly more closure—characterized by the extent of re-epithelization—compared to saline control and PDGF-B). Near 100% closure was observed 9 d post-treatment in wounds that received AREG₁₂₆₋₁₃₈/PDGF-BB, while wounds treated with saline or PDGF-BB were still largely open.

### Example 7: IL-1Ra/PIGF₁₂₃₋₁₄₁ Fusion Protein for Treating Wounds

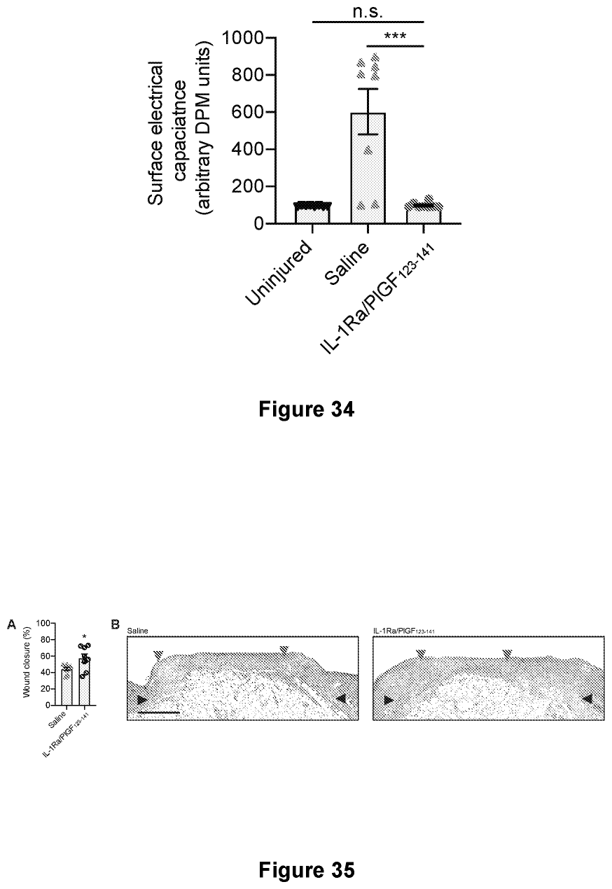

To determine the effect of the IL-1Ra/PIGF₁₂₃₋₁₄₁ fusion protein on wounds, we tested the epithelial barrier properties of IL-1Ra/PIGF₁₂₃₋₁₄₁-treated wounds 9 days post-treatment, by measuring their surface electrical capacitance. Wounds treated with saline showed high capacitance values, while wounds treated with IL-1Ra/PIGF₁₂₃₋₁₄ showed values that were similar to those measured on uninjured skin, indicating low fluid leakage and therefore reformation of an epithelial barrier (see FIG. 34).

### Example 8: IL-1Ra/PIGF₁₂₃₋₁₄₁ Fusion Protein for Treating Skin Wounds

To determine the effect of the IL-1Ra/PIGF₁₂₃₋₁₄₁ fusion protein on non-diabetic wounds, splinted full-thickness wounds in wild-type mice (C57BL/6) were treated with saline or IL-1Ra/PIGF₁₂₃₋₁₄₁ (0.61 μg). Wound closure 6 days post-treatment was evaluated by histomorphometric analysis of tissue sections as shown in FIG. 33 A. Data are means±SEM. n=8 wounds per condition. Two-tailed Welch's t test. *P≤0.05. Representative histology (haematoxylin and eosin staining) 6 d post-treatment shown in FIG. 33 B. Black arrows indicate wound edges and gray arrows indicate tips of epithelium tongue. The granulation tissue under the epithelium contains granulocytes with dark nuclei. Fat tissue appears as transparent bubbles. Scale bar=1 mm.

Compared to treatment with saline control, treatment with IL-1Ra/PIGF₁₂₃₋₁₄₁ led to a significant but modest improvement of wound closure in wild-type mice.

### REFERENCES

U.S. Pat. No. 9,879,062 B2
Mochizuki, M., et al. Growth factors with enhanced syndecan binding generate tonic signalling and promote tissue healing. Nat Biomed Eng (2019)
E. de Castro et al., “ScanProsite: detection of PROSITE signature matches and ProRule-associated functional and structural residues in proteins,” Nucleic Acids Res., vol. 34, no. Web Server issue, pp. W362-5, July 2006.

S. F. Altschul, W. Gish, W. Miller, E. W. Myers, and D. J. Lipman, “Basic local alignment search tool,” J. Mol. Biol., vol. 215, no. 3, pp. 403-10, October 1990.
M. M. Martino et al., “Growth factors engineered for super-affinity to the extracellular matrix enhance tissue healing,” Science, vol. 343, no. 6173, pp. 885-8, February 2014.
Zhang. Y (2001) PNAS, 98, 974-9.
Sierecki, E. et al., (2010) J Med Chem 53, 6899-6911.
Manning, B. D. and Cantley, L. C. (2007) Cell 129, 1261-1274.

1. A fusion protein comprising interleukin-1 receptor antagonist (IL-1Ra) and an extracellular matrix (ECM) binding peptide which specifically binds to one or more or all extracellular matrix proteins selected from the group consisting of fibrinogen, fibronectin, vitronectin, tenascin C and heparan sulfate.

2. The fusion protein of claim 1 in which the ECM binding peptide comprises a heparin binding domain of placental growth factor comprising the amino acid sequence provided as SEQ ID NO: 1 (RRRPKGRGKRRREKQRPTD) or conservative variants thereof.

3. The fusion protein of claim 1 in which the ECM binding peptide comprises a peptide from amphiregulin (AREG) comprising the amino acid sequence provided as SEQ ID NO: 2 (RKKKGGKNGKNRR) or conservative variations thereof.

4. The fusion protein of claim 1 in which the ECM binding peptide comprises a peptide from neurturin (NRTN) comprising the amino acid sequence provided as SEQ ID NO: 3 (RRLRQRRRLRRE) or conservative variations thereof.

5. The fusion protein of claim 1 comprising the ECM binding peptide linked at its N terminus to IL-1Ra either directly or indirectly via a linker.

6. The fusion protein of claim 1 in which the IL-1Ra amino acid sequence is SEQ ID NO:4 or SEQ ID NO: 5.

7. The fusion protein of claim 1 in which the ECM binding peptide has or comprises the amino acid sequence provided as any one of SEQ ID NO: 1-3 or 8-59.

8. The fusion protein of claim 1 having the amino acid sequence of SEQ ID NO: 60 or SEQ ID NO: 61.

9. A nucleic acid molecule encoding the fusion protein of claim 1 or a vector comprising a nucleic acid molecule encoding the fusion protein of claim 1 or a cell or a non human organism transformed or transfected with a nucleic acid molecule encoding the fusion protein of claim 1 or with a vector comprising a nucleic acid molecule encoding the fusion protein of claim 1.

10. A method of
(a) treating a condition in which IL-1Ra administration is beneficial or in which IL-1R1 signalling needs to be dampened,
(b) enhancing tissue regeneration, particularly bone regeneration and/or wound repair or for treating wounds, burns and muscle, cartilage, tendon and bone disorders,
(c) enhancing the regenerative activity of growth factor administration, or
(d) reducing inflammation or desensitisation of a cell to growth factor stimulation in a subject being administered a growth factor,
the method comprising administering to a subject in need thereof the fusion protein of claim 1, a nucleic acid molecule encoding the fusion protein of claim 1, or a vector comprising a nucleic acid molecule encoding the fusion protein of claim 1.

11. The method of claim 10 in which the condition in which IL-1Ra administration is beneficial or in which IL-1R1 signalling needs to be dampened is a condition requiring tissue regeneration, particularly bone regeneration and/or wound repair.

12. The method of claim 10 in which the condition in which IL-1Ra administration is beneficial or in which IL-1R1 signalling needs to be dampened is a wound, burn or muscle condition or injury or a cartilage, tendon or bone disorder or injury, particularly a diabetic wound.
